SEQUENCE LISTING (96 sequences)

SEQ ID NO: 1 - DNA, 18 bp, Artificial Sequence
  Forward primer that binds to GALNT 1 sequences
cgtcacccttccagaaat

SEQ ID NO: 2 - DNA, 18 bp, Artificial Sequence
  Reverse primer that binds to GFP sequences
ccactgcaaagcttcttc

SEQ ID NO: 3 - DNA, 19 bp, Artificial Sequence
  Forward primer that binds to SRC sequences
gctttggcgaggtgtggat

SEQ ID NO: 4 - DNA, 19 bp, Artificial Sequence
  Reverse primer that binds to SRC sequences
acatcgtgccaggcttcag

SEQ ID NO: 5 - DNA, 23 bp, Artificial Sequence
  Forward primer that binds to Src sequences
gtgagggagagtgagaccacaaa

SEQ ID NO: 6 - DNA, 21 bp, Artificial Sequence
  Reverse primer that binds to Src sequences
ggcattgtcgaagtcggatac

SEQ ID NO: 7 - DNA, 20 bp, Artificial Sequence
  Forward primer that binds to GBF1 sequences
gaggcaaggactttgagcaa

SEQ ID NO: 8 - DNA, 20 bp, Artificial Sequence
  Reverse primer that binds to GBF1 sequences
tctgctcctcaggcattaca

SEQ ID NO: 9 - DNA, 17 bp, Artificial Sequence
  Forward primer that binds to Gbf1 sequences
gctgcccaccccaaatg

SEQ ID NO: 10 - DNA, 20 bp, Artificial Sequence
  Reverse primer that binds to Gbf1 sequences
tgaagggcacaccaccagta

SEQ ID NO: 11 - DNA, 20 bp, Artificial Sequence
  Forward primer that binds to ARF1 sequences
gcttaagctgggtgagatcg

SEQ ID NO: 12 - DNA, 20 bp, Artificial Sequence
  Reverse primer that binds to ARF1 sequences
gtcccacacagtgaagctga

SEQ ID NO: 13 - DNA, 21 bp, Artificial Sequence
  Forward primer that binds to Arf1 sequences
gcgccactacttccagaacac

SEQ ID NO: 14 - DNA, 24 bp, Artificial Sequence
  Reverse primer that binds to Arf1 sequences
gctctctgtcattgctgtccacta

SEQ ID NO: 15 - DNA, 24 bp, Artificial Sequence
  Forward primer that binds to GALNT1 sequences
ctgattctcaaattccacgtcact

SEQ ID NO: 16 - DNA, 24 bp, Artificial Sequence
  Reverse primer that binds to GALNT1 sequences
gacactgattcgtttccacatttc

SEQ ID NO: 17 - DNA, 22 bp, Artificial Sequence
  Forward primer that binds to Galnt1 sequences
gacttcctgctggtgacgttct

SEQ ID NO: 18 - DNA, 20 bp, Artificial Sequence
  Reverse primer that binds to Galnt1 sequences
ccccatttctccaggacctt

SEQ ID NO: 19 - DNA, 20 bp, Artificial Sequence
  Forward primer that binds to GALNT2 sequences
gacgcctgagcagagaaggt

SEQ ID NO: 20 - DNA, 19 bp, Artificial Sequence
  Reverse primer that binds to GALNT2 sequences
cagcccaccagcaatcatg

SEQ ID NO: 21 - DNA, 16 bp, Artificial Sequence
  Forward primer that binds to Galnt2 sequences
cgcagcggtgccttct

SEQ ID NO: 22 - DNA, 22 bp, Artificial Sequence
  Reverse primer that binds to Galnt2 sequences
gctccagcctgctctgaatatt

SEQ ID NO: 23 - DNA, 21 bp, Artificial Sequence
  Forward primer that binds to GALNT3 sequences
ccacgttgcttagaactgtcc

SEQ ID NO: 24 - DNA, 21 bp, Artificial Sequence
  Reverse primer that binds to GALNT3 sequences
ccaaaatgatttccttcagca

SEQ ID NO: 25 - DNA, 23 bp, Artificial Sequence
  Forward primer that binds to Galnt3 sequences
tgaaggagatcattttggtggat

SEQ ID NO: 26 - DNA, 20 bp, Artificial Sequence
  Reverse primer that binds to Galnt3 sequences
ttcctccagcttttcatgca

SEQ ID NO: 27 - DNA, 19 bp, Artificial Sequence
  Forward primer that binds to GALNT4 sequences
gtctgattggggccacttt

SEQ ID NO: 28 - DNA, 20 bp, Artificial Sequence
  Reverse primer that binds to GALNT4 sequences
cagccaaccggaattacact

SEQ ID NO: 29 - DNA, 21 bp, Artificial Sequence
  Forward primer that binds to Galnt4 sequences
tctttcagggtttggcagtgt

SEQ ID NO: 30 - DNA, 16 bp, Artificial Sequence
  Reverse primer that binds to Galnt4 sequences
gcccacgtgcgagcat

SEQ ID NO: 31 - DNA, 20 bp, Artificial Sequence
  Forward primer that binds to GALNT5 sequences
caataacctcccaaccacca

SEQ ID NO: 32 - DNA, 20 bp, Artificial Sequence
  Reverse primer that binds to GALNT5 sequences
ggaggagagcgattgatgac

SEQ ID NO: 33 - DNA, 22 bp, Artificial Sequence
  Forward primer that binds to Galnt5 sequences
cagtggacagagccattgaaga

SEQ ID NO: 34 - DNA, 24 bp, Artificial Sequence
  Reverse primer that binds to Galnt5 sequences
tgggaggtcattgtgaactagttg

SEQ ID NO: 35 - DNA, 20 bp, Artificial Sequence
  Forward primer that binds to GALNT6 sequences
cgcaaagcagctgtgtctac

SEQ ID NO: 36 - DNA, 20 bp, Artificial Sequence
  Reverse primer that binds to GALNT6 sequences
tggctattcttgccagtgaa

SEQ ID NO: 37 - DNA, 20 bp, Artificial Sequence
  Forward primer that binds to Galnt6 sequences
gcagaggtgctcacgttcct

SEQ ID NO: 38 - DNA, 18 bp, Artificial Sequence
  Reverse primer that binds to Galnt6 sequences
ctccagccagccgtgaaa

SEQ ID NO: 39 - DNA, 20 bp, Artificial Sequence
  Forward primer that binds to GALNT7 sequences
tgcattgatagcatgggaaa

SEQ ID NO: 40 - DNA, 20 bp, Artificial Sequence
  Reverse primer that binds to GALNT7 sequences
gtggcagggtcctagttcaa

SEQ ID NO: 41 - DNA, 19 bp, Artificial Sequence
  Forward primer that binds to Galnt7 sequences
ttggcgcacagaaggctaa

SEQ ID NO: 42 - DNA, 20 bp, Artificial Sequence
  Reverse primer that binds to Galnt7 sequences
cacctcacagtgggcatcaa

SEQ ID NO: 43 - DNA, 20 bp, Artificial Sequence
  Forward primer that binds to GALNT8 sequences
tccactcttgaagccactcc

SEQ ID NO: 44 - DNA, 20 bp, Artificial Sequence
  Reverse primer that binds to GALNT8 sequences
ccctgatccaagcagacatt

SEQ ID NO: 45 - DNA, 20 bp, Artificial Sequence
  Forward primer that binds to Galnt8 sequences
ccattatacaacgggccatc

SEQ ID NO: 46 - DNA, 20 bp, Artificial Sequence
  Reverse primer that binds to Galnt8 sequences
tgagctgaaatcatccacca

SEQ ID NO: 47 - DNA, 20 bp, Artificial Sequence
  Forward primer that binds to GALNT9 sequences
aacgtgtacccggagatgag

SEQ ID NO: 48 - DNA, 20 bp, Artificial Sequence
  Reverse primer that binds to GALNT9 sequences
tccctggtccagacagtagg

SEQ ID NO: 49 - DNA, 22 bp, Artificial Sequence
  Forward primer that binds to Galnt9 sequences
agccatcctctacccctgtcat

SEQ ID NO: 50 - DNA, 21 bp, Artificial Sequence
  Reverse primer that binds to Galnt9 sequences
caggagaccttcggcactgta

SEQ ID NO: 51 - DNA, 20 bp, Artificial Sequence
  Forward primer that binds to GALNT10 sequences
tggatggatgagtacgcaga

SEQ ID NO: 52 - DNA, 20 bp, Artificial Sequence
  Reverse primer that binds to GALNT10 sequences
gctttttctggactgcgaca

SEQ ID NO: 53 - DNA, 23 bp, Artificial Sequence
  Forward primer that binds to Galnt10 sequences
ctggcataacaaggaggctatca

SEQ ID NO: 54 - DNA, 21 bp, Artificial Sequence
  Reverse primer that binds to Galnt10 sequences
ggcttcccctgttctccatat

SEQ ID NO: 55 - DNA, 20 bp, Artificial Sequence
  Forward primer that binds to GALNT11 sequences
acccaaagtccttcaacgtg

SEQ ID NO: 56 - DNA, 20 bp, Artificial Sequence
  Reverse primer that binds to GALNT11 sequences
gcatttgttggtctggaggt

SEQ ID NO: 57 - DNA, 19 bp, Artificial Sequence
  Forward primer that binds to Galnt11 sequences
agagtcctgcagcgtggaa

SEQ ID NO: 58 - DNA, 16 bp, Artificial Sequence
  Reverse primer that binds to Galnt11 sequences
ctgggccaccaggcat

SEQ ID NO: 59 - DNA, 20 bp, Artificial Sequence
  Forward primer that binds to GALNT12 sequences
tgaagcctggtcaactctcc

SEQ ID NO: 60 - DNA, 20 bp, Artificial Sequence
  Reverse primer that binds to GALNT12 sequences
gatatccggggatgtctcaa

SEQ ID NO: 61 - DNA, 21 bp, Artificial Sequence
  Forward primer that binds to Galnt12 sequences
gggatgggtcagaaccagttt

SEQ ID NO: 62 - DNA, 21 bp, Artificial Sequence
  Reverse primer that binds to Galnt12 sequences
tggcgggtgttatagcgtatt

SEQ ID NO: 63 - DNA, 21 bp, Artificial Sequence
  Forward primer that binds to GALNT13 sequences
gaagcttggagcactctcctt

SEQ ID NO: 64 - DNA, 21 bp, Artificial Sequence
  Reverse primer that binds to GALNT13 sequences
tggggaacgatttatcacact

SEQ ID NO: 65 - DNA, 23 bp, Artificial Sequence
  Forward primer that binds to Galnt13 sequences
ggctgtgcttattccaaaagatg

SEQ ID NO: 66 - DNA, 25 bp, Artificial Sequence
  Reverse primer that binds to Galnt13 sequences
gccatgaggttaaactgattgattt

SEQ ID NO: 67 - DNA, 20 bp, Artificial Sequence
  Forward primer that binds to GALNT14 sequences
ctaaagttgagcccctgtgc

SEQ ID NO: 68 - DNA, 20 bp, Artificial Sequence
  Reverse primer that binds to GALNT14 sequences
ccatacctgggactttgcat

SEQ ID NO: 69 - DNA, 21 bp, Artificial Sequence
  Forward primer that binds to Galnt14 sequences
cagaaagctttgcgcctagac

SEQ ID NO: 70 - DNA, 19 bp, Artificial Sequence
  Reverse primer that binds to Galnt14 sequences
ccctccggctatgattgga

SEQ ID NO: 71 - DNA, 20 bp, Artificial Sequence
  Forward primer that binds to GALNTL1 sequences
caaccagctggagagtgaca

SEQ ID NO: 72 - DNA, 20 bp, Artificial Sequence
  Reverse primer that binds to GALNTL1 sequences
ggtccgaggagtaggacaca

SEQ ID NO: 73 - DNA, 20 bp, Artificial Sequence
  Forward primer that binds to Galntl1 sequences
tgtgacaggaacaccctcaa

SEQ ID NO: 74 - DNA, 20 bp, Artificial Sequence
  Reverse primer that binds to Galntl1 sequences
gctgacaggtacgccttctc

SEQ ID NO: 75 - DNA, 20 bp, Artificial Sequence
  Forward primer that binds to GALNTL2 sequences
cttccaggagaatgggatga

SEQ ID NO: 76 - DNA, 20 bp, Artificial Sequence
  Reverse primer that binds to GALNTL2 sequences
ttgttttcttgcaccacagc

SEQ ID NO: 77 - DNA, 20 bp, Artificial Sequence
  Forward primer that binds to Galntl2 sequences
tacaagtggcctgcctacag

SEQ ID NO: 78 - DNA, 20 bp, Artificial Sequence
  Reverse primer that binds to Galntl2 sequences
gcctcatcatggaagcagag

SEQ ID NO: 79 - DNA, 20 bp, Artificial Sequence
  Forward primer that binds to GALNTL4 sequences
cagcgtgtacccagagatga

SEQ ID NO: 80 - DNA, 20 bp, Artificial Sequence
  Reverse primer that binds to GALNTL4 sequences
cagcactccataggcaatga

SEQ ID NO: 81 - DNA, 20 bp, Artificial Sequence
  Forward primer that binds to Galntl4 sequences
gctggaccacttggagaatg

SEQ ID NO: 82 - DNA, 20 bp, Artificial Sequence
  Reverse primer that binds to Galntl4 sequences
gcaggagcctcttggatatg

SEQ ID NO: 83 - DNA, 20 bp, Artificial Sequence
  Forward primer that binds to GALNTL5 sequences
tggatttttggggaagagaa

SEQ ID NO: 84 - DNA, 20 bp, Artificial Sequence
  Reverse primer that binds to GALNTL5 sequences
agagttggcctccacacatc

SEQ ID NO: 85 - DNA, 20 bp, Artificial Sequence
  Forward primer that binds to Galntl5 sequences
ccaaggattgaggcgatatg

SEQ ID NO: 86 - DNA, 20 bp, Artificial Sequence
  Reverse primer that binds to Galntl5 sequences
tctctctcgatgcccagtct

SEQ ID NO: 87 - DNA, 20 bp, Artificial Sequence
  Forward primer that binds to GALNTL6 sequences
acaacagccccgttacactc

SEQ ID NO: 88 - DNA, 19 bp, Artificial Sequence
  Reverse primer that binds to GALNTL6 sequences
tgttgctcacaggatggaa

SEQ ID NO: 89 - DNA, 20 bp, Artificial Sequence
  Forward primer that binds to Galntl6 sequences
atggctcggttttccaaagt

SEQ ID NO: 90 - DNA, 20 bp, Artificial Sequence
  Reverse primer that binds to Galntl6 sequences
cggatgagaccttccctttt

SEQ ID NO: 91 - DNA, 25 bp, Artificial Sequence
  Forward primer that binds to C1GALT1C1 sequences
ccttgtaaaacccaaagatgtgagt

SEQ ID NO: 92 - DNA, 23 bp, Artificial Sequence
  Reverse primer that binds to C1GALT1C1 sequences
tgtcacagtgtttggtccaagtc

SEQ ID NO: 93 - DNA, 20 bp, Artificial Sequence
  Forward primer that binds to C1galt1c1 sequences
acgccggagtatttgcagaa

SEQ ID NO: 94 - DNA, 24 bp, Artificial Sequence
  Reverse primer that binds to C1galt1c1 sequences
ccaacggatttggtattaaacaca

SEQ ID NO: 95 - DNA, 18 bp, Artificial Sequence
  Forward primer that binds to exogenous Galnt1- GFP sequences
cgtcacccttccagaaat

SEQ ID NO: 96 - DNA, 18 bp, Artificial Sequence
  Reverse primer that binds to exogenous Galnt1- GFP sequences
ccactgcaaagcttcttc

(19) United States
(12) Patent Application Publication
(10) Pub. No.: US 2020/0264186 A1
(43) Pub. Date: Aug. 20, 2020

(54) BIOMARKERS TO DETECT AND CHARACTERISE CANCER

(71) Applicant: AGENCY FOR SCIENCE, TECHNOLOGY AND RESEARCH, Singapore (SG)

(72) Inventors: Frederic Bard, Singapore (SG); Zhi Hui Joanne Chia, Singapore (SG)

(21) Appl. No.: 16/649,903

(22) PCT Filed: Oct. 4, 2018
(86) PCT No.: PCT/SG2018/050502

§ 371 Date: Mar. 23, 2020

(30) Foreign Application Priority Data
Oct. 4, 2017 (SG) 10201708183V

(51) Int. Cl. G01N 33/574

(52) U.S. Cl. CPC G01N 33/57496; G01N 2440/38; G01N 2400/02

(57) ABSTRACT

Disclosed herein are methods of detecting the presence or absence of cancer. Also disclosed herein are methods of characterising a sample from a subject thought to be suffering cancer, as well as methods of determining the malignancy, grade, or staging of a cancer. Also disclosed herein are kits utilising the methods disclosed herein.

### CROSS-REFERENCE TO RELATED APPLICATIONS

This application claims the benefit of priority of SG provisional application No. 10201708183V, filed 4 Oct. 2017, the contents of it being hereby incorporated by reference in its entirety for all purposes.

### FIELD OF THE INVENTION

The present invention relates generally to the field of molecular biology. In particular, the present invention relates to the use of biomarkers for the detection and characterisation of cancer.

### BACKGROUND OF THE INVENTION

An invasive tumour phenotype drives faster tumour growth and is often correlated with the formation of metastases and poor prognosis. For most patients with cancers, metastasis is the ultimate cause of mortality. Detection of cancers at an early stage is difficult due to the lack of sensitivity of current methods, as well as the lack of targets available to allow such detection. Most cancers can only be detected at later stages, and, sometimes, at a time when the disease is no longer curable or the symptoms no longer treatable.

Thus there is an unmet need for methods which allow early detection and characterisation of cancer.

### SUMMARY

In one aspect, the present invention refers to a method of detecting the presence or absence of cancer, wherein the method comprises the steps of (i) obtaining a sample from a subject; (ii) detecting the level of O-glycosylation of one or more endoplasmic reticulum (ER)-resident proteins in the sample obtained in step (ii); (iii) comparing the level of O-glycosylation of one or more endoplasmic reticulum (ER)-resident proteins in step (ii) with the level of O-glycosylation of one or more endoplasmic reticulum (ER)-resident proteins in a control group; wherein an increase in the level of O-glycosylation of one or more endoplasmic reticulum (ER)-resident proteins present in the sample compared to the control group is indicative of the presence of cancer.

In another aspect, the present invention refers to a method of determining the risk of a subject developing cancer, wherein the method comprises the steps of (i) obtaining a sample from a subject; (ii) detecting the level of O-glycosylation of one or more endoplasmic reticulum (ER)-resident proteins in the sample; (iii) comparing the level of O-glycosylation of one or more endoplasmic reticulum (ER)-resident proteins in step (ii) with the level of O-glycosylation of one or more endoplasmic reticulum (ER)-resident proteins in a control group; wherein an at least 4-fold increase in the level of O-glycosylation of one or more endoplasmic reticulum (ER)-resident proteins present in the sample compared to the control group is indicative that the subject is suffering from cancer.

In yet another aspect, the present invention refers to a method of determining the malignancy, grade, or staging of a cancer, the method comprising (i) obtaining a sample from a subject; (ii) detecting the level of O-glycosylation of one or more endoplasmic reticulum (ER)-resident proteins in the sample; (iii) comparing the level of O-glycosylation of one or more endoplasmic reticulum (ER)-resident proteins in step (ii) with the level of O-glycosylation of one or more endoplasmic reticulum (ER)-resident proteins in a group defined for each grade of cancer.

In a further aspect, the present invention refers to a kit comprising a monosaccharide-binding protein capable of binding to one or more O-glycosylated endoplasmic reticulum (ER)-resident proteins; a detection agent capable of binding to the monosaccharide-binding protein and/or the one or more O-glycosylated endoplasmic reticulum (ER)-resident proteins; and one or more standards, wherein each standard comprises any one of the O-glycosylated endoplasmic reticulum (ER)-resident proteins as disclosed herein.

### BRIEF DESCRIPTION OF THE DRAWINGS

The invention will be better understood with reference to the detailed description when considered in conjunction with the non-limiting examples and the accompanying drawings, in which:

FIG. 1 is a schematic drawing that illustrates N-acetylgalactosamine (GalNAc)-T Activation Pathway (GALA) activation. During GALA activation, polypeptide N-acetylgalactosaminyltransferases (GALNTs) relocate from the Golgi to the endoplasmic reticulum (ER), which leads to increased O-glycosylation of proteins in the ER, including matrix metalloproteinase-14 (MMP14) and protein disulfide isomerase family A member 4 (PDIA4). O-glycosylated matrix metalloproteinase-14 (MMP14) leads to increased extra-cellular matrix (ECM) degradation. Cells with high GALA activation during early stage cancer can lead to rapid tumour growth, neighbouring organ invasion and metastasis during late stage cancer. Conversely, cells with low GALA activation during early stage cancer can lead to slow tumour growth, rare invasion and rare metastasis during late stage cancer.

FIG. 2 presents data showing results that malignant liver tumours display high Tn staining and glycosylation of the ER-resident protein PDIA4. (A) is a vertical scatter plot that shows the quantification of Tn antigen using the intensity of Vicia villosa lectin (VVL) antibody staining levels during human liver tumour progression. Tissue microarrays (TMA) of human liver biopsies, LV8011 and BC03002 that include normal, benign, malignant (with different grades), and metastatic liver tumours were analysed. Horizontal lines indicate the mean of each group. (B) shows four images that are close-up images of the representative cores in the tissue microarray (TMA) BC03002 that is stained with Vicia villosa lectin (VVL) antibody. Scale bar, 100 μm. (C) shows two images that are immunohistofluorescence analysis of Tn using Helix pomatia lectin (HPL) antibodies to stain mouse hepatocellular carcinoma (HCC). Scale bar, 100 μm. Asterisks mark background staining of red blood cells. (D) shows six images that show co-staining of Vicia villosa lectin (VVL) and ER-marker Calnexin in mouse hepatocellular carcinoma (HCC) and normal tissue. Scale bar, 10 μm. Nuclei stained using Hoechst. (E) is an image of a Western blot showing the immunoblot analysis of the level of Tn modified ER-resident PDIA4 in two normal mouse livers and three NRas-G12V/shp53-injected mouse tumour samples at early (6 weeks post-injection; 6 wpi) stage and four tumour samples from the late (24 weeks post-injection; 24 wpi) stage. Cell lysates were immunoprecipitated with Vicia villosa lectin (VVL) and probed for PDIA4. The numbers indicate liver samples from different mice. (F) is an image of two Western blots representing the immunoblot analysis of the level of Tn modified ER-resident PDIA4 in HEK cells stimulated with growth factors epidermal growth factor (EGF) and platelet-derived growth factor (PDGF) over 2, 4 and 6 hours. (G) is an image of a Western blot showing the immunoblot analysis of the levels of Tn modified PDIA4 in 20 pairs of human hepatocellular carcinoma (HCC) tumours (T) versus patient-matched normal liver tissues (NT) from 20 random hepatocellular carcinoma (HCC) patients. (H) is a scatter plot showing the quantification of the levels of Tn modified PDIA4 normalized by total PDIA4 in human hepatocellular carcinoma (HCC) tumours in the western blot shown in FIG. 1G. The fold change with respect to the corresponding normal liver is presented. (I) shows a heat map depicting the results of quantitative reverse transcription polymerase chain reaction (qRT-PCR) assessment of 19 polypeptide N-acetylgalactosaminyltransferase (GALNT) family members associated with liver tumour progression. Clustering analysis represented as heat maps shows the expression differences between liver tumours and adjacent non-cancerous tissues for 22 livers from patients.

FIG. 3 presents data showing the expression of ER-targeted polypeptide N-acetylgalactosaminyltransferase 1 (GALNT1) drives rapid tumour progression. (A) is a schematic diagram of the Sleeping Beauty (SB) transposon system used with three plasmids under the control of the PGK promoter: one encoding Sleeping Beauty (SB) transposase, the second harbouring mCherry-Nras, and the third expressing shp53 and a gene of interest (GOI) fused to EGFP. The last two plasmids are flanked by inverted repeat (IR) sequences, allowing for genomic insertion. (B) is a line graph representing a Kaplan-Meier survival curve of mice after injection with plasmids from (A) encoding GFP or GFP-tagged forms of wild-type Galnt1 (Golgi-G1), ER-localized Galnt1 (ER-G1), or ER-G1 catalytically inactive (ER-G1ΔCat). Statistical significance was calculated with log rank relative to GFP. (C) is a column graph representing the average number and size of gross nodules per mouse at 6 weeks post injection (wpi), n=9 for each group. Error bars indicate standard deviation (SD). *p<0.005 (t-test). (D) shows images showing histopathological and immunohistochemical (IHC) analyses of livers from GFP, Golgi-G1, and ER-G1 groups at 6 weeks post injection (wpi). Representative images of the gross livers from GFP, Golgi-G1, and ER-G1 groups are on the left panels. Black arrows indicate tumour nodules (Left, n=4 for each group; scale bar, 1 cm). Representative images of histopathological hematoxylin and eosin (H&E) staining and immunohistochemical (IHC) analyses for Vicia villosa lectin (VVL), GFP and mCherry (Right; scale bar, 100 μm) of livers from GFP, Golgi-G1, and ER-G1 groups at 6 weeks post injection (wpi). H: liver hyperplasia, HA: hepatocellular adenoma, HCC: hepatocellular carcinoma. Liver lesions outlined by dotted lines.

FIG. 4 presents data showing how ER-G1 promotes tumour growth at an early stage. (A) shows images of immunohistochemical (IHC) staining of liver sections from NRas-G12V/shp53-EGFP mice at 3 days post-injection (dpi), n=3. Scale bars, 100 μm. (B) shows images of immunohistochemical (IHC) staining of liver sections from NRas-G12V/shp53-EGFP-Golgi-G1 mice at 3 days post-injection (dpi), n=3. Scale bars, 100 μm. (C) shows images of immunohistochemical (IHC) staining of liver sections from NRas-G12V/shp53-EGFP-ER-G1 mice at 3 days post-injection (dpi), n=3. Scale bars, 100 μm. (D) is a column graph depicting results of the analysis of quantification of positive cells per visual field (10×) on each liver section with three mice per group: mCherry- and GFP-expressing cells. Student's t-test was calculated relative to GFP and error bars indicate standard deviation (SD). NS, not significant. (E) is a column graph depicting results of the analysis of quantification of positive cells per visual field (10×) on each liver section with three mice with Vicia villosa lectin (VVL)-expressing cells. Student's t-test was calculated relative to GFP and error bars indicate standard deviation (SD). (F) shows images of immunohistochemical (IHC) staining for mCherry of NRas-G12V/shp53-EGFP-, Golgi-G1- or ER-G1-expressing cells in mouse livers at 7 days post-injection (dpi). Scale bar, 100 μm. (G) is a vertical box plot showing the quantification of the area of mCherry expressing cells from three different mouse livers at 3 days post-injection (dpi), n=9 for each group. p values shown are relative to control GFP-injected mice (t-test). Lines indicate the mean of each group. (H) is a vertical box plot representing the quantification of the area of mCherry expressing cells from three different mouse livers at 7 days post-injection (dpi), n=9 for each group. p values shown are relative to control GFP-injected mice (t-test). Lines indicate the mean of each group. (I) is a graph representing the growth rate of HepG2 GFP, Golgi-G1 and ER-G1 cell lines over time. The percentage confluence in the wells was acquired every 6 hours. Student's t-test was calculated relative to HepG2 GFP cells and error bars indicate standard error of the mean (SEM) of three replicates. NS, not significant.

FIG. 5 presents data showing that ER-G1 promotes liver tumour invasiveness. (A) is a table showing that the number and percentage of mice from the GFP, WT-G1 and ER-G1 groups that have shown metastasis into lung, spleen, pancreas, muscle, kidney and stomach at time of death. (B) shows representative images of metastasis in lung tissue from ER-G1 mice stained using hematoxylin and eosin (H&E), anti-Vicia villosa lectin (VVL) and anti-GFP. Scale bar, 100 μm. (C) shows representative images of liver tumour (T) invading the pancreas (P) stained using hematoxylin and eosin (H&E), anti-Vicia villosa lectin (VVL) and anti-GFP, with zoomed images on the right. Invasive tumour nodules (T) are outlined by a dotted line. Scale bar, 100 μm. (D) is a vertical scatter plot showing results of an analysis of the percentage of GFP⁺ circulating tumour cells (CTCs) (n=4 per group) at time of death. Line indicates the mean in each group. Student's t-test was calculated relative to GFP mice shown. (E) is a line graph representing results of the analysis of results from the in vitro Förster resonance energy transfer-matrix metalloproteinase (FRET-MMP) substrate cleavage assay in mouse liver tissues. The numbers indicate liver samples from different mice of each condition. Values on graph indicate the mean±standard error of the mean (SEM) from triplicate measurement of the same liver sample, *p<0.05, **p<0.001, and ***p<0.0001 relative to normal liver sample (t-test). (F) is a vertical scatter plot showing results of the analysis of matrix metalloproteinases (MMP) substrate cleavage activity at 140 minutes time point of mouse liver lysates from (E). The lines indicate the mean in each group. *p<0.05 relative to lysate from normal liver (t-test). NS, not significant. (G) is a line graph representing the quantification of matrix metalloproteinases 14 (MMP14) activity in cell lysates from HepG2 GFP, Golgi-G1, and ER-G1 cell lines based on in vitro cleavage of a Förster resonance energy transfer-matrix metalloproteinase (FRET-MMP) substrate peptide. Values on graph indicate the mean±standard error of the mean (SEM) from triplicate measurement, *p<0.05, **p<0.001, and ***p<0.0001 relative to HepG2 GFP cell line (t-test). (H) shows representative images of HepG2 GFP (control), Golgi-G1 and ER-G1 cells seeded on fluorescently labelled gelatin sheets in a gelatin degradation assay. Scale bar, 10 μm. (I) is a column graph representing the quantification of the area of gelatin degradation, ***p<0.0001 relative to ER-G1 (t-test). Values on the graph indicate the mean±standard error of the mean (SEM) from three replicate wells.

FIG. 6 presents data showing that O-glycosylation of matrix metalloproteinase-14 (MMP14) is required for cellular ECM degradation. (A) shows representative images of gelatin degradation assay for small interfering ribonucleic acid (siRNA) knockdown with two different matrix metalloproteinase-14 (MMP14) small interfering ribonucleic acid (siRNA) sequences (siGenome [siG] and On-targetplus [OnT]) and non-targeting (NT) small interfering ribonucleic acid (siRNA) in HepG2 ER-G1 cells. Scale bar, 20 μm. (B) is a column graph showing the quantification of gelatin degradation assay for small interfering ribonucleic acid (siRNA) knockdown with two different matrix metalloproteinase-14 (MMP14) small interfering ribonucleic acid (siRNA) sequences (siGenome [siG] and On-targetplus [OnT]) and non-targeting (NT) small interfering ribonucleic acid (siRNA) in HepG2 ER-G1 cells. Values indicate the mean±standard deviation (SD) from two replicates, *p<0.05 relative to HepG2 GFP cells (t-test). (C) shows representative images of HepG2 GFP and ER-G1 cells expressing matrix metalloproteinase-14 (MMP14) wild-type seeded on fluorescently labelled collagen/gelatin matrix layer in a collagen/gelatin layer degradation assay. Scale bar, 10 μm. (D) is a column graph showing the quantification measurement of collagen/gelatin layer degradation assay of HepG2 GFP and ER-G1 cells expressing matrix metalloproteinase-14 (MMP14) wild-type. Values indicate the mean±standard error of the mean (SEM) from three replicates. ***p<0.0001 relative to GFP (t-test). (E) is a schematic representation of O-glycosylation sites on matrix metalloproteinase-14 (MMP14). N-acetylgalactosamine (GalNAc) sugar residues are indicated by the dark grey boxes. (F) is an image of a Western blot showing immunoblot analysis of matrix metalloproteinase-14 (MMP14) levels from a Vicia villosa lectin (VVL) immunoprecipitation (IP) of multiple NRas-G12V/shp53/ERG1-injected mouse liver samples as well as normal liver samples and samples from two different NRas-G12V/shp53-EGFP-injected mice. Vicia villosa lectin (VVL), matrix metalloproteinase-14 (MMP14) and actin levels were also analysed the cell lysate, wherein actin was used a loading control. (G) is an image of a Western blot representing Tn modification levels of matrix metalloproteinase-14 (MMP14) in multiple NRas-G12V/shp53-injected mouse tumour samples at early (6 weeks post injection (wpi)) and late (24 weeks post injection (wpi)) stages compared to normal mouse livers. The samples used here were the same samples used in FIG. 2E. The numbers indicate liver samples from different mice. Actin was used as loading control. (H) an image of a Western blot representing an immunoblot analysis of the levels of Tn modification on wild type (WT) MMP14-mCherry and various matrix metalloproteinase-14 (MMP14) mutants transfected in HepG2 Cosmc^{-/-} cell lines expressing Golgi-G1 and ER-G1. MMP14-T(4)A refers to mutant form of matrix metalloproteinase-14 (MMP14) bearing four alanine substitutions, T299A-T300A-S301A-S304A. MMP14-T(5)A refers to mutant form of matrix metalloproteinase-14 (MMP14) bearing five alanine substitutions, T291A-T299A-T300A-S301A-S304A. Cell lysates were immunoprecipitated using red fluorescent protein (RFP) beads to isolate MMP14-mCherry; Tn modifications (proprotein, active and cleaved) were observed with Vicia villosa lectin (VVL) staining. (I) is an image of a Western blot representing the immunoblot analysis of the levels of extended O-glycans on MMP14-V5 in HepG2 cells expressing GFP control, Golgi-G1, or ER-G1. Cell lysates were immunoprecipitated using peanut agglutinin (PNA) or Datura stramonium Lectin (DSL) lectins and probed with V5 tag antibody for matrix metalloproteinase-14 (MMP14). (J) shows representative images of HepG2 ER-G1 cells expressing matrix metalloproteinase-14 (MMP14) wild-type (MMP-WT) and various matrix metalloproteinase-14 (MMP14) mutants, MMP14-T291A, MMP14-T(4)A, MMP14-T(5)A and MMP14-E240A seeded on fluorescently labelled collagen/gelatin matrix layer in a collagen/gelatin layer degradation assay. Scale bar, 10 μm. (K) is a column graph representing the quantification of the area of gelatin degradation by HepG2 ER-G1 cells expressing various matrix metalloproteinase-14 (MMP14) mutants. Values on the graph indicate the mean±standard error of the mean (SEM) from three replicates, ***p<0.05 relative to HepG2 ER-G1 cells expressing matrix metalloproteinase-14 (MMP14) WT (t-test). NS, not significant.

FIG. 7 presents data showing matrix metalloproteinase-14 (MMP14) glycosylation is required for liver tumour growth and metastasis. (A) is a line graph representing the Kaplan-Meier survival curve of mice injected with NRas-G12V/shp53-ER-G1 with and without shMMP14, a short hairpin ribonucleic acid (shRNA) against matrix metalloproteinase-14 (MMP14). Statistical significance calculated with log rank relative to GFP control. (B) is a vertical scatter plot representing analysis of the percentage of GFP⁺ circulating tumour cells (CTCs) in the bloodstream of mice from (A) (n=3 per group). Horizontal lines indicate the mean in each group. Student's t-test relative to control mice as well as ER-G1 co-expressing shMMP14 mice was calculated. (C) is a table showing the percentage and number of mice injected with NRas-G12V/shp53-ER-G1 with and without shMMP14 that have shown invasion and metastasis into lung, spleen, pancreas, skin, kidney and stomach. (D) shows representative immunohistochemical (IHC) staining images of matrix metalloproteinase-14 (MMP14) and mCherry in mouse livers injected with various Sleeping Beauty (SB) constructs at 7 days post-injection (dpi). Magnified images of matrix metalloproteinase-14 (MMP14) staining are shown on the right panels. Scale bars, 100 μm. (E) is a vertical scatter plot showing the quantification of the area occupied by mCherry-expressing cells in the various injected mouse livers shown in (D) at 7 days post-injection (dpi). Horizontal lines indicate the mean in each group. Student's t-test relative to ER-G1 as well as ER-G1 co-expressing matrix metalloproteinase-14 (MMP14) livers was calculated.

FIG. 8 presents data showing high Tn expression in both human and mouse hepatocellular carcinoma (HCC). (A) shows representative immunohistochemical (IHC) images of Vicia villosa lectin (VVL) staining of human tissue microarrays BC03002 and LV8011 that cover the liver disease spectrum. N: normal, In: Inflammation or Hepatitis, H: Hyperplasia, HCA: Hepatocellular adenoma, HCC: Hepatocellular carcinoma with the numeric 1, 2, 3 representing different tumour grades, C: Intrahepatic cholangiocarcinoma. (B) is a column graph representing the quantification of Vicia villosa lectin (VVL) intensity between benign hepatocellular adenoma (HCA) and malignant hepatocellular carcinoma (HCC) after normalisation to normal livers, n=4 mice per group. (C) shows representative images of Tn staining of normal, hepatocellular adenoma (HCA) and hepatocellular carcinoma (HCC) grade 2-3 tissues, wherein hepatocellular carcinoma (HCC) grade 2-3 tissue showed intensive Tn staining as compared to normal liver and hepatocellular adenoma (HCA). A zoom in image of the section of the tissue are in the top left corner of each image. Scale bar, 1 mm. (D) shows representative close-up images of the cores of the normal liver and hepatocellular carcinoma (HCC) grade 2 tissues shown in (A), scale bar: 50 μm. (E) is a heat map depicting the expression patterns of 19 genes of the polypeptide N-acetylgalactosaminyltransferase (GALNT) family members using 12 Nras/shp53-SB mouse liver samples including non-cancerous liver, hepatocellular adenoma (HCA) and hepatocellular carcinoma (HCC). (F) is a Venn diagram showing the up-regulated and down-regulated genes specific to hepatocellular carcinoma (HCC) in both human and mice, wherein the significantly differentially expressed genes identified in human and mouse liver tumours has fold-change ≥1.5 and P≤0.05 as compared to normal tissue. (G) is a Venn diagram showing up-regulated and down-regulated genes in hepatocellular adenoma (HCA) and hepatocellular carcinoma (HCC) in mice. (H) is an image of a Western blot showing the immunoblot analysis of VVL and Tn modified PDIA levels in 20 pairs of human hepatocellular carcinoma (HCC) tumours (T) versus patient-matched normal liver tissues (NT) from 20 random hepatocellular carcinoma (HCC) patients. The 20 pairs of patient samples are denoted by numbers F009, F012, F016, F017, F019, F022, F025, F026, F028, F031, F034, F037, F038, F039, F040, F042, F046, F049, F052, F074 and F036. Actin is used as loading control.

FIG. 9 presents data showing the assessment of exogenous GALNT1 expression in mouse liver samples and the comparison of Nras and ER-G1 as drivers of liver tumorigenesis. (A) is a vertical scatter plot showing relative transcription levels of exogenous Galnt1 determined by quantitative RT-PCR using a set of primers (SEQ ID NOs. 1 and 2) in GFP, Golgi-G1 and ER-G1 mice. Log 2 fold-change were calculated against an internal housekeeeping genes (β-actin). (B) shows an image showing a sodium dodecyl sulfate polyacrylamide gel electrophoresis (SDS-PAGE) analysis of GFP and Galnt1-GFP levels in normal mouse livers and livers 3 days post-injection of the respective transposon constructs Nras-G12V/shp53-GFP, -Golgi-G1 and ERG1. Actin was used as loading control. (C) is a schematic showing the work flow on ImageJ to quantify the area of Sleeping Beauty (SB) transposon transformed cells in the livers of post-one week injected mice. (D) shows photos that are representative images of cells in the livers of Sleeping Beauty (SB) transposon transformed GFP, Golgi-G1 and ER-G1 groups 1 week post injection (wpi). The images on the right panel show the masking (light grey) in the Sleeping Beauty (SB) transposon transformed GFP, Golgi-G1 and ER-G1 groups, using the workflow from (C). Scale bar, 100 μm. (E) is a line graph representing a log-rank survival curve analysis of two groups of mice injected with plasmid expressing Sleeping Beauty transposase together with either Nras/shp53 or ER-G1/shp53 plasmid. (F) is a column graph representing the number of tumours with average nodules >0.5 cm³ per mice upon death. No liver tumours were found in mice expressing ER-G1/shp53 as compared to mice expressing Nras/shp53, which displayed approximately 1±3 nodules larger than 0.5 cm³ upon death. (G) shows representative images of immunohistochemical analyses of livers collected from Nras/shp53 and ER-G1/shp53 groups at 40 weeks post-injection (wpi). Left panel shows representative images of Tn levels using Vicia villosa lectin (VVL) staining in Nras/shp53 versus ER-G1/shp53 livers, with black boxes reflecting the zoomed in images of different stainings presented in the three right panels. Zoomed in images of liver section from ER-G1/shp53 show hematoxylin and eosin (H&E), Vicia villosa lectin (VVL) and EGFP staining. Zoomed in images of liver section from Nras/shp53 show hematoxylin and eosin (H&E), Vicia villosa lectin (VVL), and mCherry staining. Scale bar, 100 μm.

FIG. 10 presents data representing the establishment of stable HepG2 cell lines expressing various constructs. (A) shows representative immunofluorescence staining images of the ER resident protein calnexin and ER-G1 in HepG2 ER-G1 cell line. Scale bar, 20 μm. (B) shows representative images of GFP and Helix pomatia lectin (HPL) staining of HepG2-GFP, Golgi-G1 and ER-G1 cell lines. Scale bar, 30 μm. (C) is a column graph representing the quantification of Helix pomatia lectin (HPL) staining of HepG2-GFP, Golgi-G1 and ER-G1 cell lines. Values on the graph indicate the mean±standard error of the mean (SEM). **P<0.001 and ***P<0.0001, relative to HepG2-GFP cells. (D) is an image showing sodium dodecyl sulfate polyacrylamide gel electrophoresis (SDS-PAGE) analysis of protein expression levels of each construct in HepG2-GFP, Golgi-G1 and ER-G1 cell lines. The upper bands indicate Golgi-G1 and ER-G1 constructs while the lower bands at 30 kDa indicate the control GFP protein. Actin levels are used as loading control.

FIG. 11 presents representative images of how ER-G1 enhances liver tumour invasion and metastasis into various organs. (A) shows representative images of serial sections of ER-G1 liver tumour invading into the diaphragm stained for hematoxylin and eosin (H&E), with a black box reflecting the zoomed in image. Zoomed in images show hematoxylin and eosin (H&E), Vicia villosa lectin (VVL) or EGFP staining. (B) shows representative images of serial sections of ER-G1 liver tumour invading into the spleen stained for hematoxylin and eosin (H&E), with a black box reflecting the zoomed in image. Zoomed in images show hematoxylin and eosin (H&E), Vicia villosa lectin (VVL) or EGFP staining. (C) shows hematoxylin and eosin (H&E) staining that represent ER-G1 tumours attached and invaded into kidney, with a black box reflecting the zoomed in image. Zoomed in images show hematoxylin and eosin (H&E) staining. White arrow indicates a renal capsule. (D) shows hematoxylin and eosin (H&E)-stained ER-G1 tumour, with a black box reflecting the zoomed in image. Zoomed in images show hematoxylin and eosin (H&E) staining, and the white arrow shows the serosal surface, wherein the ER-G1 tumour has invaded into the stomach through the serosal surface. (E) shows hematoxylin and eosin (H&E)-stained invasive ER-G1 tumour on the top panel, with a black box reflecting the zoomed in image. The bottom panels show a zoomed in image of ER-G1 tumour invading into the skin via the stratum basal. Scale bar, 100 μm.

FIG. 12 presents data showing ER-G1 enhances matrix degradation in HepG2 cells via matrix metalloproteinase-14 (MMP14) glycosylation. (A) is an image representing the sodium dodecyl sulfate polyacrylamide gel electrophoresis (SDS-PAGE) analysis of matrix metalloproteinase-14 (MMP14) levels in NT5, MMP14-siG and MMP14-onT treated HepG2-GFP, Golgi-G1 and ER-G1 cells. MMP14-siG and MMP14-onT are matrix metalloproteinase-14 (MMP14) small interfering ribonucleic acid (siRNA). Actin levels are used as loading controls. (B) is an image representing the sodium dodecyl sulfate polyacrylamide gel electrophoresis (SDS-PAGE) analysis of MMP14-V5 transfected cell lines HepG2-GFP, Golgi-G1 and ER-G1, after 72 hours of metabolic incorporation with artificial sugar GalNAz, an O-glycan with an azide-modified analog of N-acetylgalactosamine (GalNAc) that can be modified by click chemistry and conjugated to a FLAG peptide when incorporated into a glycoprotein. Lysates were immunoprecipitated with FLAG antibody to isolate all O-GalNAz modified proteins and probed with V5 antibody for matrix metalloproteinase-14 (MMP14). FLAG, Vicia villosa lectin (VVL), V5 and actin antibodies were used to analyse the levels of O-GalNAz modified proteins, 0-glycosylated proteins, matrix metalloproteinase-14 (MMP14) and actin respectively in the cell lysates of the same MMP14-V5 transfected cell lines HepG2-GFP, Golgi-G1 and ER-G1. (C) is a column graph showing the levels of GalNAz modified matrix metalloproteinase-14 (MMP14) using the immunoprecipitation method with FLAG antibody from (B). Values on the graph indicate the mean±standard deviation (SD) from two replicates, * P<0.05. (D) is a schematic of O-glycan structures in the Golgi and ER, wherein the O-glycan structures are recognized by various lectins such as Datura stramonium Lectin (DSL), peanut agglutinin (PNA) and Helix pomatia lectin (HPL). (E) is an image representing the sodium dodecyl sulfate polyacrylamide gel electrophoresis (SDS-PAGE) analysis of endogenous matrix metalloproteinase-14 (MMP14), Tn and actin levels in HepG2-GFP, -Golgi-G1 and -ER-G1 cell lines using MMP14, Vicia villosa lectin (VVL) and actin antibodies. The endogenous matrix metalloproteinase-14 (MMP14) is represented by two bands, wherein the upper band represents the matrix metalloproteinase-14 (MMP14) proprotein, and the lower band represents the active matrix metalloproteinase-14 (MMP14). (F) is an image representing the sodium dodecyl sulfate polyacrylamide gel electrophoresis (SDS-PAGE) analysis of the MMP14-V5 constructs MMP14-WT, MMP14-T291A, MMP14-T(4)A, MMP14-T(5)A and MMP14-E240A that are transiently transfected HepG2-ER-G1 cells. V5 and actin antibodies are used to analyse matrix metalloproteinase-14 (MMP14) and actin respectively. (G) shows representative immunofluorescence staining images of cell surface matrix metalloproteinase-14 (MMP14) and Helix pomatia lectin (HPL) in HepG2-GFP and -ER-G1 cells. Scale bar, 10 μm. (H) is a column graph showing the quantification immunofluorescence staining of cell surface matrix metalloproteinase-14 (MMP14) levels of GFP, Golgi-G1, ER-G1, ER-G1, ER-G1 treated with NT5 small interfering ribonucleic acid (siRNA), ER-G1 treated with MMP14-siG, and ER-G1 treated with MMP14-onT. Values on the graph indicate the mean±standard deviation (SD) from three replicates, * P<0.05 and ** P<0.001. (I) shows representative images of MMP14-mcherry, Helix pomatia lectin (HPL) and GFP staining in HepG2 GFP and ER-G1 cell lines. Scale bar, 10 μm.

FIG. 13 presents data showing glycosylation of matrix metalloproteinase-14 (MMP14) by ER-G1 increases its substrate cleavage activity. (A) is a line graph depicting data of in vitro Förster resonance energy transfer-matrix metalloproteinase (FRET-MMP) substrate cleavage assay in lysates of HepG2 cells expressing GFP (light grey solid line) and ER-G1 with wild type MMP14 (black solid line) and mutants MMP14-T291A (dark grey dotted line) and -T(5)A (black dotted line). Lysis buffer (dark grey solid line) is used as a control. ***P<0.0001 relative to ERG1 co-expressing with wild-type MMP14. (B) is a Western blot image showing the expression levels of endogenous matrix metalloproteinase-14 (MMP14) in normal mouse livers, and mouse livers at 1 week post injection of N-Ras/p53 (n=3), ER-G1 (n=2) and ER-G1 with shMMP14 (n=3), a short hairpin ribonucleic acid (shRNA) against matrix metalloproteinase-14 (MMP14). Actin was used as a loading control. (C) shows representative images of immunohistochemical (IHC) staining of matrix metalloproteinase-14 (MMP14) of mouse livers at 1 weeks post injection with GFP+MMP14, ER-G1+MMP14 and ER-G1+MMP14-T(5)A. Scale bar, 50 μm.

FIG. 14 presents data showing glycosylation of ER-resident proteins in a mouse liver cancer model. (A) is a Western blot image showing the immunoblot analysis of glycosylation of ER-resident proteins PDIA4, PDIA3, CANX, HSPA5 and ERLEC1 in normal liver samples and tumour samples at 6 weeks post-injection (6 wpi) and 24 weeks post-injection (24 wpi). (B) is a vertical scatter plot that shows the quantification of the levels of glycosylated ER-resident proteins with respect to that in normal liver (1), as shown in (A). 6 weeks post-injection (6 wpi) corresponds to early stage tumour and 24 weeks post-injection (24 wpi) corresponds to late stage tumour. (C) is a Western blot image showing the expression of VVL and CANX in ER-GALNT1 inducible cells. Human liver HepG2 cells that stably expresses a doxycycline (Dox) inducible form of ER-targeted GALNT1 are used, wherein uninduced cells represent GALA negative cells and doxycycline (Dox) induced cells represent GALA positive cells, wherein the expression of ER-GALNT1 mimics GALA activation. Doxycycline (Dox) induced cells show a 6.5-fold increase of level of glycosylated ER-resident protein CANX.

FIG. 15 presents data showing glycosylation of ER-resident proteins in 20 pairs of human liver tumours. (A) is a Western blot image showing the immunoblot analysis of glycosylation of ER-resident proteins PDIA4 and CANX in 20 pairs of human hepatocellular carcinoma (HCC) tumours (T) versus patient-matched normal liver tissues (NT) from 20 random hepatocellular carcinoma (HCC) patients. The 20 pairs of patient samples are denoted by numbers F009, F012, F016, F017, F019, F022, F025, F026, F028, F031, F034, F037, F038, F039, F040, F042, F046, F049, F052, F074 and F036. (B) is a vertical scatter plot that shows the levels of glycosylated PDIA4 in the tumours with respect to the Edmondson Grade. Values on each point represent the ratio of the Tn modified PDIA4 in the tumour with respect to that in the corresponding normal tissues from a single patient. (C) is a vertical scatter plot that shows the levels of glycosylated CANX in the tumours with respect to the Edmondson Grade. Values on each point is normalised to corresponding normal tissues.

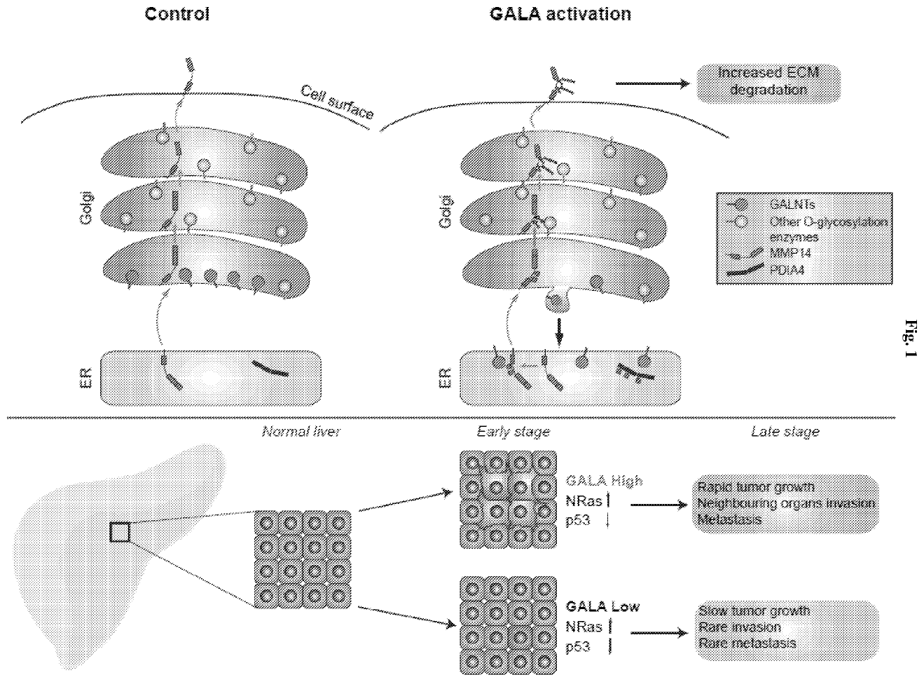

### DETAILED DESCRIPTION

Many cancers are associated with invasive phenotypes, usually resulting in lethal outcomes. Generally speaking, the later the stage of the disorder, the more serious the symptoms are. While some disorders can be detected early, it is difficult to detect the disorders at the early stages due to the lack of sensitivity of current methods, as well as the lack of targets or biomarkers available to allow such detection. Most of the disorders can only be detected at later stages, and sometimes when the symptoms are no longer treatable or the disease incurable.

Glycosylation is frequently altered in cancer. Protein glycosylation is heavily modified in cancer, where cell-surface glycosylated proteins dictate how cancer cells interact with surrounding tissue and proliferate. Invasiveness also correlates with perturbed O-glycosylation, a covalent modification of cell-surface proteins.

For example and without being bound by theory, it is thought that an invasive tumour phenotype drives faster tumour growth, and is often correlated with the formation of metastases and poor prognosis.

Cancer, for example, can be a devastating disease with high mortality rates, especially at the later stages. For most cancer patients, metastasis is the ultimate cause of mortality. The molecular mechanisms that cause cancers to grow within tissues remain unclear. An invasive tumour phenotype drives faster tumour growth and is often correlated with the formation of metastases and poor prognosis.

One such example is liver cancer, wherein the invasive phenotype is correlated to intra-liver metastases and usually a lethal outcome. Liver cancer is rising in incidence and currently the sixth most common and second-leading cause of cancer-related deaths worldwide. This high mortality arises because of the difficulty associated with the early diagnosis of liver cancer, combined with a lack of effective chemotherapeutic treatments and a tendency for tumours to metastasize both locally and into other organs, rendering surgical recession ineffective. While methods are currently available to diagnose cancer, the accuracy and efficacy of these methods remain to be proven. In addition, there is a lack of methods to effectively detect cancers in the early stages.

Thus, in one aspect, there is disclosed a method of detecting the presence or absence of a cancer. Also disclosed herein are methods for determining the risk of a subject developing cancer, and methods for method of determining the malignancy of a disorder, for example cancer. The methods disclosed herein are based on the use of biomarkers as disclosed herein for determining the presence of absence of the diseases described herein.

In one example, the determination of the presence or absence of a disorder comprises detecting the level of O-glycosylation of one or more endoplasmic reticulum (ER)-resident proteins in the sample obtained from a subject. In another example, the detected levels are compared to levels of the same targets in a control group. In yet another example, the increase in the level of O-glycosylation of one or more endoplasmic reticulum (ER)-resident proteins is indicative of the presence of a disorder. In another example, the decrease in the level of O-glycosylation of one or more endoplasmic reticulum (ER)-resident proteins is indicative of the absence of a disorder.

As used herein, the terms “disorder” and “disease” can be used interchangeably, and refer to an undesirable condition or syndrome, wherein a more or less specific set of symptoms have been identified by clinicians. The method disclosed herein can be used to detect one or more of the diseases as disclosed herein.

In one example, the disorder is cancer. For example, the cancer is, but is not limited to, liver cancer, breast cancer, lung cancer, hepatocellular carcinoma (HCC), hepatocellular adenoma (HCA), fibrolamellar hepatocellular carcinoma (FHCC), hepatoblastoma, focal nodular hyperplasia (FNH), nodular regenerative hyperplasia, ductal carcinoma in situ (DCIS), Paget's disease of the breast, comedocarcinoma, invasive ductal carcinoma (IDC), intraductal papilloma, lobular carcinoma in situ (LCIS), invasive lobular carcinoma (ILC), medullary carcinoma, inflammatory breast cancer, non-small cell lung cancer (NSCLC) and small cell lung cancer (SCLC). In another example, the disorder is liver cancer. In yet another example, the disorder is hepatocellular carcinoma (HCC) or hepatocellular adenoma (HCA).

Thus in example, there is disclosed a method of staging or characterising the identified disorder based on the subject matter disclosed herein. In one example, the method of determining the malignancy, grade, or staging of a cancer comprises obtaining a sample from a subject; detecting the level of O-glycosylation of one or more endoplasmic reticulum (ER)-resident proteins in the sample; comparing the level of O-glycosylation of one or more endoplasmic reticulum (ER)-resident proteins with the level of O-glycosylation of one or more endoplasmic reticulum (ER)-resident proteins in a group defined for each grade of cancer.

In another example, the cancer is benign or malignant. In another example, the cancer can be characterised by staging, for example, stage 0, stage 1, stage 2, stage 3 or stage 4. In a further example, the cancer is staged according to the Edmondson Grade.

As used herein, the term “Edmondson Grade”, also known as the Edmondson and Steiner grading system (ESGS), refers to a grading system for tumours based on histopathology of samples obtained from a subject. The grading definition according to the Edmondson grade is as follows: Grade I consists of small tumour cells, arranged in trabeculae, with abundant cytoplasm and minimal nuclear irregularity that are almost indistinguishable from normal liver tissue. Grade II tumours have prominent nucleoli, hyperchromatism, and some degree of nuclear irregularity. Grade III tumours show more pleomorphism than grade II, and have angulated nuclei. Grade IV has prominent pleomorphism and often anaplastic giant cells. A table of the histological features based on the Edmondson and Steiner grading system are provided below.

| | | | | Other |
|---|---|---|---|---|
| Grades | | Architecture | Cytology | features |
| I | Well | Thin trabecular, | Minimal atypia | Fatty change |
| | differentiated | frequent acinar | | is frequent |
| | | structures | | |
| II | Moderately | Trabecular (3 or | Abundant | Bile or |
| | differentiated | more cells in | eosinophilic | proteinaceous |
| | | thickness) and | cytoplasm, | fluid within |
| | | acinar | round nuclei | acini |
| | | | with distinct | |
| | | | nucleoli | |
| III | Poorly | Solid | Moderate to | Absence of |
| | differentiated | | marked | sinusoid-like |
| | | | pleomorphism | blood spaces |
| IV | Undifferentiated | Solid | Little | n.a. |
| | | | cytoplasm, | |
| | | | spindle, or | |
| | | | round-shaped | |
| | | | cells | |

In addition, staging can be used, and is required, to determine how advanced the cancer is in a patient. One current method for cancer staging includes use of the TNM staging system, wherein T describes the size of the primary tumour and if the primary tumour has metastasised to nearby tissues; N describes if the lymph nodes contain the cancer cells; and M refers to the presence of metastasis to distant parts of the body. However, such method of cancer staging is somewhat inefficient as it is done by clinical or pathologic observations by clinicians or pathologists, wherein such observations are dependent on the quality of samples obtained during biopsies. Ambiguities can arise if the quality of the biopsy sample is poor, or when there is insufficient difference in prognosis between pathologic stages of the disease, resulting in inaccurate staging, which in turn can lead to insufficient therapy.

Detection and characterisation the diseases disclosed herein is performed on a sample. As used herein, the term “sample” refers to a specimen taken, obtained or derived from a subject. In one example, the sample is obtained from a subject. In another example, the sample is a biological sample. For example, the sample is, but is not limited to, biopsy of a subset of tissues, cells or component parts, or a fraction or portion thereof; whole blood or a component thereof (e.g. plasma, serum); urine, saliva lymph, bile fluid, sputum, tears, cerebrospinal fluid, bronchoalveolar lavage fluid, synovial fluid, semen, ascitic tumour fluid, breast milk, pus, amniotic fluid, buccal smear, cultured cells, culture medium collected from cultured cells, cell pellet, a lysate, homogenate or extract prepared from a whole organism or a subset of its tissues, cells, or component parts, or a fraction or portion thereof. In one example, the sample can be cells isolated from an organ from an organism, wherein the organ can be, but is not limited to, liver, brain, heart, spleen, kidney, bone, lymph nodes, muscles, blood vessels, bone marrow, pancreas, intestines, urinary bladder, or skin. In another example, the sample can be cells isolated from the joint from an organism, wherein the cells can be from, but is not limited to, cartilage, bone, muscle, ligament, tendon, connective tissue, or any combinations thereof.

As used herein, the term “subject” refers to an animal, preferably a mammal, which is the object of administration, treatment, observation or experiment. Mammal includes, but is not limited to, humans and both domestic animals such as laboratory animals and household pets, for example, but not limited to, cats, dogs, swine, cattle, sheep, goats, horses, rabbits, and non-domestic animals such as, but not limited to, wildlife, fowl, birds and the like. In one example, the mammal is a rodent, for example, but not limited to, mouse and rat. In yet another example, the mammal is a human.

The method disclosed herein is based on the so-called N-acetylgalactosamine (GalNAc)-T activation (GALA) pathway, which has been identified to be activated in disorders, such as, but not limited to, cancer.

As used herein, the terms “GALA”, “GalNAc-T activation pathway” or “GALA pathway” are used interchangeably throughout and refer to the process of relocation of polypeptide N-acetylgalactosaminyltransferases (GALNTs) from the Golgi to the endoplasmic reticulum. This results in an increase of O-glycosylation and Tn antigen levels in the endoplasmic reticulum, as well as overall increase in protein glycosylation.

As used herein, the term “O-glycosylation” refers to the post-translational modification process of attaching a mono-, or polysaccharide molecule, or a glycan, to an amino acid residue in a protein. This attachment is performed at an oxygen atom present in the amino acid to which the glycan is to be attached. In one example, O-linked glycans can be attached to the hydroxyl oxygen of, for example, serine, threonine, tyrosine, hydroxylysine, or hydroxyproline side-chains, or to oxygen atoms on lipids such as, but not limited to, ceramide phosphoglycans linked through the phosphate of a phosphoserine. The process of glycosylation usually takes place within the Golgi apparatus in eukaryotes, and can affect cell signalling pathways, thereby leading to changes in biological processes and functional changes in the cell. Thus, in one example, the method disclosed herein relies on the O-glycosylation of proteins in order to determine the presence or absence of a disease.

The enzymes involved in the process of glycosylation are usually referred to as glycosyltransferase, which are enzymes which establish glycosidic linkages. In other words, the glycosyltransferase attaches the saccharide molecule (also known as a “glycosyl donor”) to a (nucleophilic) glycosyl acceptor molecule, which is usually an oxygen-, carbon-, nitrogen- or sulphur-based molecule.

As used herein, the term “glycan” refers to refers to compounds consisting of a large number of monosaccharides linked glycosidically. That is to say that the monosaccharides are linked between the hemiacetal or the hemiketal group of one saccharide and the hydroxyl group of another compound.

The term “glycan”, as used herein, is used synonymously with the term polysaccharide. Glycans can be homo- or heteropolymers of monosaccharide residues, and can be linear or branched. In general, glycans are found on the exterior surface of cells, whereby O- and N-linked glycans are very common in eukaryotes. For example, glycans can comprise solely of O-glycosidic linkages of monosaccharides. In another example, the glycan is, but is not limited to, N-acetylgalactosamine (GalNAc), N-acetylglucosamine, fucose, glucose, xylose, galactose, mannose, or any combinations thereof. In one example, the glycan is an O-linked glycan.

In one example, where the glycosyl donor is N-acetyl-galactosamine, the enzyme which catalyses the linkage of N-acetyl-galactosamine to the glycosyl acceptor molecule is a polypeptide N-acetylgalactosaminyltransferase. In another example, the O-glycan O-GalNAc is formed when N-acetylgalactosamine (GalNAc) is bound to the hydroxyl group of serine or threonine in a protein, a reaction which is catalysed by GALNT.

As used herein, the terms “O-linked glycan” and “O-glycan” are used interchangeably throughout and refer to glycans that are attached to a protein through serine or threonine residues. In another example, the O-glycan is O—N-acetylgalactosamine (O-GalNAc) linked to serine or threonine in a protein. In another example, the O-glycan is Tn. As used herein, the term “Tn antigen” or “Tn” are used interchangeably throughout and refer to O-GalNAc.

As used herein, the term “N-acetylgalactosamine” or “GalNAc” are used interchangeably throughout, and refer to the monosaccharide that is involved in the O-glycosylation process. As mentioned above, for example, GalNAc is linked to a hydroxyl group of the amino acids serine or threonine in a protein during O-glycosylation by, for example, GALNTs, leading to the formation of O-linked N-acetylgalactosamine (O-GalNAc).

As used herein, the term “polypeptide N-acetylgalactosaminyltransferases” or “GALNTs” are used interchangeably throughout and refer to a glycosyltransferase enzyme that catalyses the transfer of a N-acetylgalactosamine to the hydroxyl group of the amino acids serine or threonine in a protein during O-glycosylation. In one example, the polypeptide N-acetylgalactosaminyltransferases (GALNTs) can be, but is not limited to, polypeptide N-acetylgalactosaminyltransferase 1 (GALNT 1), polypeptide N-acetylgalactosaminyltransferase 2 (GALNT 2), polypeptide N-acetylgalactosaminyltransferase 3 (GALNT 3), polypeptide N-acetylgalactosaminyltransferase 4 (GALNT 4), polypeptide N-acetylgalactosaminyltransferase 5 (GALNT 5), polypeptide N-acetylgalactosaminyltransferase 6 (GALNT 6), polypeptide N-acetylgalactosaminyltransferase 7 (GALNT 7), polypeptide N-acetylgalactosaminyltransferase 8 (GALNT 8), polypeptide N-acetylgalactosaminyltransferase 9 (GALNT 9), polypeptide N-acetylgalactosaminyltransferase 10 (GALNT 10), polypeptide N-acetylgalactosaminyltransferase 11 (GALNT 11), polypeptide N-acetylgalactosaminyltransferase 12 (GALNT 12), polypeptide N-acetylgalactosaminyltransferase 13 (GALNT 13), polypeptide N-acetylgalactosaminyltransferase 14 (GALNT 14), polypeptide N-acetylgalactosaminyltransferase 15 (GALNT 15), polypeptide N-acetylgalactosaminyltransferase 16 (GALNT 16), polypeptide N-acetylgalactosaminyltransferase 17 (GALNT 17), polypeptide N-acetylgalactosaminyltransferase 18 (GALNT 18), polypeptide N-acetylgalactosaminyltransferase like 5 (GALNTL5), polypeptide N-acetylgalactosaminyltransferase like 6 (GALNTL6), or any combinations thereof.

The term “protein”, “peptide” and “polypeptide”, as used herein, are used interchangeably throughout and refer to a molecule comprising two or more amino acid residues joined to each other by peptide bonds. A protein may also be just a fragment of a naturally occurring protein or peptide. A protein can be wild-type, mutated, recombinant, naturally occurring, or synthetic and may constitute all or part of a naturally-occurring, or non-naturally occurring polypeptide. A protein or peptide must contain at least two amino acids and no limitation is placed on the maximum number of amino acids which may comprise a protein's or peptide's sequence. In one example, the protein can be an enzyme. As used herein, the term “enzyme” is a protein that can catalyse a biochemical reaction. The reaction can be naturally occurring or non-naturally occurring. In another example, the protein can be modified by post-translational modifications.

As used herein, the term “post-translational modification” refers to chemical modification of proteins, wherein the chemical modification can be catalysed by an enzyme. For example, post-translational modification can be, but is not limited to O-glycosylation, N-glycosylation, acetylation, methylation, phosphorylation, ubiquitylation, sulfation, hydroxylation, amidation, or any combinations thereof.

In another example, the protein can be found in one or more cell compartments, for example, but not limited to, endoplasmic reticulum (ER), Golgi, cisternae, nucleus, cytoplasm, mitochondria, or any combinations thereof. In a further example, the protein can found in the endoplasmic reticulum. Such proteins are also known as an endoplasmic reticulum (ER)-resident proteins.

As used herein, the term “endoplasmic reticulum (ER)-resident protein” refers to a protein that is retained in the endoplasmic reticulum after protein folding, and is only present in the endoplasmic reticulum. The endoplasmic reticulum (ER)-resident protein disclosed herein can be found in the smooth endoplasmic reticulum and/or rough endoplasmic reticulum. In one example, the one or more endoplasmic reticulum (ER)-resident proteins are located in the lumen and/or membrane of the endoplasmic reticulum.

In order to prevent proteins from being secreted into the cell nucleus, proteins which are present in, for example, the endoplasmic reticulum have been shown to comprise a specific N-terminal or C-terminal signal sequence, thereby enabling the retention of the proteins having this signal sequence in the endoplasmic reticulum. In one example, the endoplasmic reticulum (ER)-resident protein comprises either a KDEL and/or KKXX peptide sequence. In another example, the KDEL and/or KKXX peptide sequence can be found at either the N-terminus or C-terminus of the protein. In another example, the subcellular distribution of a protein can be seen using imaging methods, for example immunofluorescent microscopy, thereby enabling the determination of whether a protein is an endoplasmic reticulum (ER)-resident protein.

According to the method disclosed herein, the endoplasmic reticulum-resident proteins are identified using known methods in the art. In another example, the endoplasmic reticulum (ER)-resident protein can be, but is not limited to, UDP-glucose ceramide glucosyltransferase-like 1 (UGGT1), chromosome 2 open reading frame 30 (ERLEC1), glycosyltransferase 25 domain containing 1 (COLGALT1/GLT25D1), hypothetical gene supported by AF216292; NM_005347; heat shock 70 kDa protein 5 (glucose-regulated protein, 78 kDa) (HSPA5/GRP78/Bip), low density lipoprotein receptor-related protein associated protein 1 (LRPAP1), osteosarcoma amplified 9 endoplasmic reticulum associated protein (OS9), prolyl 4-hydroxylase, alpha polypeptide I (P4HA1), prolyl 4-hydroxylase, beta polypeptide (P4HB), protein disulfide isomerase family A member 3 (PDIA3), protein disulfide isomerase family A member 4 (PDIA4), stromal cell-derived factor 2-like 1 (SDF2L1), sulfatase modifying factor 2 (SUMF2), thioredoxin domain containing 5 (endoplasmic reticulum); muted homolog (mouse) (TXNDC5), asparagine-linked glycosylation 9, alpha-1,2-mannosyltransferase homolog (S. cerevisiae) (ALG9), aspartate beta-hydroxylase (ASPH), calnexin (CANX), calsyntenin 1 (CLSTN1), cytoskeleton-associated protein 4 (CKAP4), emopamil binding protein (sterol isomerase) (EBP), gamma-glutamyl carboxylase (GGCX), inositol 1,4,5-triphosphate receptor type 2 (ITPR2), lectin, mannose-binding, 1 (LMAN1/ERGIC53), leprecan-like 1 (P3H2/LEPREL1), leucine proline-enriched proteoglycan (leprecan) 1 (P3H1/LEPRE1), mannosidase, alpha, class 1B, member 1 (MAN1B1), melanoma inhibitory activity family, member 3 (MIA3), mesoderm development candidate 2 (MESDC2), multiple coagulation factor deficiency 2 (MCFD2), nucleobindin 2 (NUCB2), prolyl 4-hydroxylase, transmembrane (endoplasmic reticulum) (P4HTM), prostaglandin F2 receptor negative regulator (PTGFRN), protein kinase C substrate 80K-H (PRKCSH), ribophorin I (RPN1), sel-1 suppressor of lin-12-like (C. elegans) (SEL1L), signal recognition particle receptor, B subunit (SRPRB), thioredoxin domain containing 11 (TXNDC11), tyrosylprotein sulfotransferase 2 (TPST2), and xylosyltransferase II (XYLT2). In one example, the one or more endoplasmic reticulum (ER)-resident proteins is, but is not limited to, protein disulfide isomerase family A member 4 (PDIA4), calnexin (CANX), protein disulfide isomerase family A member 3 (PDIA3), Endoplasmic Reticulum Lectin 1 (ERLEC1) and heat shock 70 kDa protein 5 (glucose-regulated protein, 78 kDa) (HSPA5/GRP78/Bip), or combinations thereof. In another example, the endoplasmic reticulum (ER)-resident protein is protein disulfide isomerase family A member 4 (PDIA4). In a further example, the endoplasmic reticulum (ER)-resident protein is calnexin (CANX). In yet another example, the endoplasmic reticulum (ER)-resident protein is protein disulfide isomerase family A member 3 (PDIA3). In another example, the endoplasmic reticulum (ER)-resident protein is Endoplasmic Reticulum Lectin 1 (ERLEC1). In one example, the endoplasmic reticulum (ER)-resident protein is heat shock 70 kDa protein 5 (glucose-regulated protein, 78 kDa) (HSPA5/GRP78/Bip).

In one example, the endoplasmic reticulum (ER)-resident proteins are any of the following combinations: protein disulfide isomerase family A member 4 (PDIA4) and calnexin (CANX); protein disulfide isomerase family A member 4 (PDIA4) and protein disulfide isomerase family A member 3 (PDIA3); protein disulfide isomerase family A member 4 (PDIA4) and Endoplasmic Reticulum Lectin 1 (ERLEC1); protein disulfide isomerase family A member 4 (PDIA4) and heat shock 70 kDa protein 5 (glucose-regulated protein, 78 kDa) (HSPA5/GRP78/Bip); calnexin (CANX) and protein disulfide isomerase family A member 3 (PDIA3); calnexin (CANX) and Endoplasmic Reticulum Lectin 1 (ERLEC1); calnexin (CANX) and heat shock 70 kDa protein 5 (glucose-regulated protein, 78 kDa) (HSPA5/GRP78/Bip); protein disulfide isomerase family A member 3 (PDIA3) and Endoplasmic Reticulum Lectin 1 (ERLEC1); protein disulfide isomerase family A member 3 (PDIA3) and heat shock 70 kDa protein 5 (glucose-regulated protein, 78 kDa) (HSPA5/GRP78/Bip); or Endoplasmic Reticulum Lectin 1 (ERLEC1) and heat shock 70 kDa protein 5 (glucose-regulated protein, 78 kDa) (HSPA5/GRP78/Bip).

In another example, the endoplasmic reticulum (ER)-resident proteins are any of the following combinations: protein disulfide isomerase family A member 4 (PDIA4), calnexin (CANX) and protein disulfide isomerase family A member 3 (PDIA3); protein disulfide isomerase family A member 4 (PDIA4), calnexin (CANX) and Endoplasmic Reticulum Lectin 1 (ERLEC1); protein disulfide isomerase family A member 4 (PDIA4), calnexin (CANX), and heat shock 70 kDa protein 5 (glucose-regulated protein, 78 kDa) (HSPA5/GRP78/Bip); protein disulfide isomerase family A member 4 (PDIA4), protein disulfide isomerase family A member 3 (PDIA3), and Endoplasmic Reticulum Lectin 1 (ERLEC1); protein disulfide isomerase family A member 4 (PDIA4), protein disulfide isomerase family A member 3 (PDIA3), and heat shock 70 kDa protein 5 (glucose-regulated protein, 78 kDa) (HSPA5/GRP78/Bip); protein disulfide isomerase family A member 4 (PDIA4), protein disulfide isomerase family A member 3 (PDIA3), and Endoplasmic Reticulum Lectin 1 (ERLEC1); protein disulfide isomerase family A member 4 (PDIA4), protein disulfide isomerase family A member 3 (PDIA3), and heat shock 70 kDa protein 5 (glucose-regulated protein, 78 kDa) (HSPA5/GRP78/Bip); protein disulfide isomerase family A member 4 (PDIA4), Endoplasmic Reticulum Lectin 1 (ERLEC1), and heat shock 70 kDa protein 5 (glucose-regulated protein, 78 kDa) (HSPA5/GRP78/Bip); calnexin (CANX), protein disulfide isomerase family A member 3 (PDIA3) and Endoplasmic Reticulum Lectin 1 (ERLEC1); calnexin (CANX), Endoplasmic Reticulum Lectin 1 (ERLEC1) and heat shock 70 kDa protein 5 (glucose-regulated protein, 78 kDa) (HSPA5/GRP78/Bip); calnexin (CANX), protein disulfide isomerase family A member 3 (PDIA3) and heat shock 70 kDa protein 5 (glucose-regulated protein, 78 kDa) (HSPA5/GRP78/Bip); or protein disulfide isomerase family A member 3 (PDIA3), Endoplasmic Reticulum Lectin 1 (ERLEC1) and heat shock 70 kDa protein 5 (glucose-regulated protein, 78 kDa) (HSPA5/GRP78/Bip).

In a further example, the endoplasmic reticulum (ER)-resident proteins are any of the following combinations: protein disulfide isomerase family A member 4 (PDIA4), calnexin (CANX), protein disulfide isomerase family A member 3 (PDIA3), and Endoplasmic Reticulum Lectin 1 (ERLEC1); calnexin (CANX), protein disulfide isomerase family A member 3 (PDIA3), Endoplasmic Reticulum Lectin 1 (ERLEC1) and heat shock 70 kDa protein 5 (glucose-regulated protein, 78 kDa) (HSPA5/GRP78/Bip); protein disulfide isomerase family A member 4 (PDIA4), protein disulfide isomerase family A member 3 (PDIA3), Endoplasmic Reticulum Lectin 1 (ERLEC1) and heat shock 70 kDa protein 5 (glucose-regulated protein, 78 kDa) (HSPA5/GRP78/Bip); protein disulfide isomerase family A member 4 (PDIA4), calnexin (CANX), Endoplasmic Reticulum Lectin 1 (ERLEC1) and heat shock 70 kDa protein 5 (glucose-regulated protein, 78 kDa) (HSPA5/GRP78/Bip); or protein disulfide isomerase family A member 4 (PDIA4), calnexin (CANX), protein disulfide isomerase family A member 3 (PDIA3), and heat shock 70 kDa protein 5 (glucose-regulated protein, 78 kDa) (HSPA5/GRP78/Bip).

As used herein, the term “biomarker” refers to molecular indicators of a specific biological property, a biochemical feature or facet that can be used to determine the presence or absence and/or severity of a particular disease or condition. In the present disclosure, the term “biomarker” refers to a protein, a fragment or variant of such a protein being associated to a disorder. In one example, the biomarker can be a gene involved in the GALA pathway. In another example, the biomarker is an O-glycosylated protein. In another example, the biomarker is an O-glycosylated ER-resident protein as disclosed herein.

In one example, it is envisaged that the biomarkers as disclosed herein are capable of detecting or diagnosing or predicting the likelihood of a patient or subject having a disorder. Accordingly, the biomarkers as disclosed herein can be incorporated in methods of detecting, methods of determining the risk, methods of prognosis for staging, diagnostic kits to determine the likelihood of a patient or subject having a disorder or prognostic kits to determine the stage of the disorder of a patient or a subject.

In one example, there is provided a method to detect the presence or absence of a disorder.

In one example, the method to detect the presence or absence of a disorder comprises the steps of a. obtaining a sample from a subject; b. detecting the level of one or more biomarkers in a sample obtained in step a.; c. comparing the level of one or more biomarkers in step b. with the level of one or more biomarkers in a control group.

The method of the present disclosure comprises the steps of a. obtaining a sample from a subject; b. detecting the level of O-glycosylation of one or more endoplasmic reticulum (ER)-resident proteins in the sample obtained in step a.; c. comparing the level of O-glycosylation of one or more endoplasmic reticulum (ER)-resident proteins in step b. with the level of O-glycosylation of one or more endoplasmic reticulum (ER)-resident proteins in a control group.

In another example, there is disclosed a method to determine the risk of a subject developing a disorder. In another example, the method further comprises the steps of obtaining a sample from a subject; detecting the level of one or more biomarkers in the sample; and comparing the level of one or more biomarkers with the level of the same biomarkers in a control group.

In one example, the method to determine the risk of a subject developing a disorder comprises the steps of the steps of a. obtaining a sample from a subject; b. detecting the level of O-glycosylation of one or more endoplasmic reticulum (ER)-resident proteins in the sample obtained in step a.; c. comparing the level of O-glycosylation of one or more endoplasmic reticulum (ER)-resident proteins in step b. with the level of O-glycosylation of one or more endoplasmic reticulum (ER)-resident proteins in a control group.

In order to detect the level of one or more biomarkers in a sample, conventional methods can be employed, including, but not limited to, methods for capturing and/or detecting one or more biomarkers in a sample. For example, methods to capture the one or more biomarkers in a sample include, but not limited to, affinity purification, immunoprecipitation, co-immunoprecipitation, chromatin immunoprecipitation, ribonucleoproteins immunoprecipitation, or any combinations thereof, have been used to precipitate proteins and protein complexes. Methods to detect the one or more biomarkers in a sample can include, but is not limited to, immunohistochemistry (IHC), immunodetection assays, fluorescence assays, immunostaining, colorimetric protein assays, or any combinations thereof.

In another example, the detection of the level of one or more biomarkers in a sample optionally comprises a step of contacting a sample with a monosaccharide-binding protein.

In another example, the detection of the level of O-glycosylation of one or more endoplasmic reticulum (ER)-resident proteins optionally comprises a step of contacting a sample with a monosaccharide-binding protein.

In one example, the monosaccharide-binding protein can be free-floating or can be immobilised to a solid surface. In another example, the monosaccharide-binding protein can be, but is not limited to, N-acetylgalactosamine binding protein, mannose binding protein, galactose binding protein, N-acetylglucosamine binding protein, N-acetylneuraminic acid binding protein or fucose binding protein. In another example, the monosaccharide-binding protein is a lectin. In another example, the monosaccharide-binding protein is N-acetylgalactosamine binding protein.

Examples of N-acetylgalactosamine binding protein include, but are not limited to, Vicia villosa lectin (VVL), Helix pomatia lectin A (HPL), Datura stramonium Lectin (DSL), ricin (RCA), peanut agglutinin (PNA) and jacalin (AIL). In another example, the N-acetylgalactosamine binding protein is either Vicia villosa lectin (VVL) or Helix pomatia lectin A (HPL).

Comparison between the diseased and disease-free samples is made based on the differences in the levels of the biomarkers in the sample obtained from a subject and the levels of the same biomarkers in the control group. Based on this comparison, the presence or the absence of a disease can be determined based on the presence or absence of the biomarkers. In one example, the presence of the biomarker indicates the presence of the disease. In another example, the absence of the biomarker indicates the disease.

Another comparison can also be made between the levels of the biomarkers in the sample obtained from a subject and the levels of the same biomarkers in the control group based on the differential expression of the biomarker. In one example, the up-regulation of the biomarker indicates the presence of a disease. In another example, the down-regulation indicates the presence of a disease. In a further example, the up-regulation of the biomarker indicates the absence of a disease. In yet another example, the down-regulation of a biomarker indicates the absence of a disease. In another example, a decrease in the level of the biomarker is indicative of the presence of a disorder. It will also be appreciated that during the activation of the GALA pathway, polypeptide N-acetylgalactosaminyltransferases (GALNTs) can be seen to relocate into the endoplasmic reticulum (ER). Without being bound by theory, it is thought that this subcellular relocation of the GALNT enzymes leads to an increased level of O-glycosylation of proteins in the endoplasmic reticulum (ER), for example, but not limited to, endoplasmic reticulum (ER)-resident protein. Therefore, in another example, an increase in the level of O-glycosylation of one or more endoplasmic reticulum (ER)-resident proteins is indicative of the presence of a disorder.

Quantitative comparisons using fold changes in the levels of the biomarkers in the sample when compared to the levels of the biomarkers in the control group can be used to determine the risk of a subject developing a disorder and indicate that the subject is suffering from a disorder. In one example, fold change increase in the levels of the biomarkers in the sample is indicative that the subject is suffering from a disorder. In one example, the increase can be, but is not limited to, about 1.5 fold, about 2-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, about 10-fold, about 10.5-fold, about 11-fold, about 11.5-fold, about 12-fold, about 12.5-fold, about 13-fold, about 13.5-fold, about 14-fold, about 14.5-fold, about 15-fold, about 15.5 fold, about 16-fold, about 16.5-fold, about 17-fold, about 17.5-fold, about 18-fold, about 18.5-fold, about 19-fold, about 19.5-fold, or about 20-fold to be indicative that the subject is suffering from a disorder. In another example, the increase can be about 1.5-fold to about 20-fold. In another example, the increase can be, but is not limited to, about 1.5-fold to about 2.3-fold, about 2.0-fold to about 2.8-fold, about 2.5-fold to about 3.3-fold, about 3.0-fold to about 3.8-fold, about 3.5-fold to about 4.3-fold, about 4.0-fold to about 4.8-fold, about 4.5-fold to about 5.3-fold, about 5.0-fold to about 5.8-fold, about 5.5-fold to about 6.3-fold, about 6.0-fold to about 6.8-fold, about 6.5-fold to about 7.3-fold, about 7.0-fold to about 7.8-fold, about 7.5-fold to about 8.3-fold, about 8.0-fold to about 8.8-fold, about 8.5-fold to about 9.3-fold, about 9.0-fold to about 9.8-fold, about 9.5-fold to about 10.3-fold, about 10.0-fold to about 10.8-fold, about 10.5-fold to about 11.3-fold, about 11.0-fold to about 11.8-fold, about 11.5-fold to about 12.3-fold, about 12.0-fold to about 12.8-fold, about 12.5-fold to about 13.3-fold, about 13.0-fold to about 13.8-fold, about 13.5-fold to about 14.3-fold, about 14.0-fold to about 14.8-fold, about 14.5-fold to about 15.3-fold, about 15.0-fold to about 15.8-fold, about 15.5-fold to about 16.3-fold, about 16.0-fold to about 16.8-fold, about 16.5-fold to about 17.3-fold, about 17.0-fold to about 17.8-fold, about 17.5-fold to about 18.3-fold, about 18.0-fold to about 18.8-fold, about 18.5-fold to about 19.3-fold, about 19.0-fold to about 19.8-fold, about 19.5-fold to about-20 fold, to be indicative that the subject is suffering from a disorder. In another example, the increase can be, but is not limited to, at least about 1.5 fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 10.5-fold, at least about 11-fold, at least about 11.5-fold, at least about 12-fold, at least about 12.5-fold, at least about 13-fold, at least about 13.5-fold, at least about 14-fold, at least about 14.5-fold, at least about 15-fold, at least about 15.5 fold, at least about 16-fold, at least about 16.5-fold, at least about 17-fold, at least about 17.5-fold, at least about 18-fold, at least about 18.5-fold, at least about 19-fold, at least about 19.5-fold, or at least about 20-fold to be indicative that the subject is suffering from a disorder. In another example, the increase in the level of O-glycosylation of one or more endoplasmic reticulum (ER)-resident proteins present in the sample is between 2-fold to 20-fold. In yet another example, the increase in the level of O-glycosylation of one or more endoplasmic reticulum (ER)-resident proteins present in the sample is at least 4-fold to be indicative that the subject is suffering from a disorder.

In one example, fold change decrease in the levels of the biomarkers in the sample is indicative that the subject is suffering from a disorder. In one example, the decrease can be, but is not limited to, about 1.5 fold, about 2-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, about 10-fold, about 10.5-fold, about 11-fold, about 11.5-fold, about 12-fold, about 12.5-fold, about 13-fold, about 13.5-fold, about 14-fold, about 14.5-fold, about 15-fold, about 15.5 fold, about 16-fold, about 16.5-fold, about 17-fold, about 17.5-fold, about 18-fold, about 18.5-fold, about 19-fold, about 19.5-fold, or about 20-fold to be indicative that the subject is suffering from a disorder. In another example, the decrease can be about 1.5-fold to about 20-fold. In another example, the decrease can be, but is not limited to, about 1.5-fold to about 2.3-fold, about 2.0-fold to about 2.8-fold, about 2.5-fold to about 3.3-fold, about 3.0-fold to about 3.8-fold, about 3.5-fold to about 4.3-fold, about 4.0-fold to about 4.8-fold, about 4.5-fold to about 5.3-fold, about 5.0-fold to about 5.8-fold, about 5.5-fold to about 6.3-fold, about 6.0-fold to about 6.8-fold, about 6.5-fold to about 7.3-fold, about 7.0-fold to about 7.8-fold, about 7.5-fold to about 8.3-fold, about 8.0-fold to about 8.8-fold, about 8.5-fold to about 9.3-fold, about 9.0-fold to about 9.8-fold, about 9.5-fold to about 10.3-fold, about 10.0-fold to about 10.8-fold, about 10.5-fold to about 11.3-fold, about 11.0-fold to about 11.8-fold, about 11.5-fold to about 12.3-fold, about 12.0-fold to about 12.8-fold, about 12.5-fold to about 13.3-fold, about 13.0-fold to about 13.8-fold, about 13.5-fold to about 14.3-fold, about 14.0-fold to about 14.8-fold, about 14.5-fold to about 15.3-fold, about 15.0-fold to about 15.8-fold, about 15.5-fold to about 16.3-fold, about 16.0-fold to about 16.8-fold, about 16.5-fold to about 17.3-fold, about 17.0-fold to about 17.8-fold, about 17.5-fold to about 18.3-fold, about 18.0-fold to about 18.8-fold, about 18.5-fold to about 19.3-fold, about 19.0-fold to about 19.8-fold, about 19.5-fold to about-20 fold, to be indicative that the subject is suffering from a disorder. In another example, the decrease can be, but is not limited to, at least about 1.5 fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, at least about 10-fold, at least about 10.5-fold, at least about 11-fold, at least about 11.5-fold, at least about 12-fold, at least about 12.5-fold, at least about 13-fold, at least about 13.5-fold, at least about 14-fold, at least about 14.5-fold, at least about 15-fold, at least about 15.5 fold, at least about 16-fold, at least about 16.5-fold, at least about 17-fold, at least about 17.5-fold, at least about 18-fold, at least about 18.5-fold, at least about 19-fold, at least about 19.5-fold, or at least about 20-fold to be indicative that the subject is suffering from a disorder.

As used herein, the term “control group” refers to a sample that does not have the disorder. In one example, the control group can be a sample obtained from a healthy volunteer or disease-free subject. As used herein, the term “disease-free” refers to being void of the undesirable condition or syndrome, wherein a subject and/or a sample can be referred to as being disease-free. Thus, in one example, the levels of a marker in a sample are compared to the levels of the same markers in a control group. In another example, the control group is a disease-free group. In another example, the control group can be a sample obtained from a subject free of cancer. In another example, the control group can be a sample obtained from a subject free of, but is not limited to, liver cancer, breast cancer, lung cancer, hepatocellular carcinoma (HCC), hepatocellular adenoma (HCA), fibrolamellar hepatocellular carcinoma (FHCC), hepatoblastoma, focal nodular hyperplasia (FNH), nodular regenerative hyperplasia, ductal carcinoma in situ (DCIS), Paget's disease of the breast, comedocarcinoma, invasive ductal carcinoma (IDC), intraductal papilloma, lobular carcinoma in situ (LCIS), invasive lobular carcinoma (ILC), medullary carcinoma, inflammatory breast cancer, non-small cell lung cancer (NSCLC) and small cell lung cancer (SCLC). In yet another example, the disease-free sample can be a non-tumour match obtained from a subject suffering from a disorder.

As used herein, the terms “non-tumour” and “non-tumour match” refer to a sample that is free of the disorder obtained from a subject suffering from a disorder. For example, a non-tumour match can be, but is not limited to, the normal tissues or cells that are found around or near the cancer cells within the same organ. In yet another example, the control group can be a non-tumour match obtained from a subject suffering from cancer. In yet another example, the control group can be a non-tumour match obtained from a subject suffering from, but is not limited to, liver cancer, breast cancer, lung cancer, hepatocellular carcinoma (HCC), hepatocellular adenoma (HCA), fibrolamellar hepatocellular carcinoma (FHCC), hepatoblastoma, focal nodular hyperplasia (FNH), nodular regenerative hyperplasia, ductal carcinoma in situ (DCIS), Paget's disease of the breast, comedocarcinoma, invasive ductal carcinoma (IDC), intraductal papilloma, lobular carcinoma in situ (LCIS), invasive lobular carcinoma (ILC), medullary carcinoma, inflammatory breast cancer, non-small cell lung cancer (NSCLC) and small cell lung cancer (SCLC).

In one example, the detection and/or comparison can be made using one or more biomarkers. In another example, the detection and/or comparison can be made using, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 biomarkers. In one example, the detection and/or comparison can be made using the level of O-glycosylation of, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 of the O-glycosylated endoplasmic reticulum (ER)-resident proteins in a sample.

In one example, the detection and/or comparison can be made using the level of, but is not limited to, 1, 2, 3, 4, 5, 6, or 7 of the polypeptide N-acetylgalactosaminyltransferases (GALNTs) in a sample.

Also disclosed herein is a kit comprising the biomarkers and components needed in order to perform the methods as described herein. In one example, the kit comprises a monosaccharide-binding protein capable of binding to one or more biomarkers.

In one example, the kit comprises a binding protein capable of binding to one or more biomarkers, wherein the binding protein is free-floating or is immobilised to a solid surface. In one example, the binding protein is an antibody or a conjugated antibody. In another example, the binding protein comprises one or more tags at the 5′ or 3′ end of said protein. Such tags can be used to, for example, detect or isolate and purify the attached molecules. Thus, a person skilled in the art would know and be able to use similar tags to attain the result provided above. These tags can be, but are not limited to, biotin, streptavidin, phosphate, histidine FLAG, triple FLAG tag (3×FLAG), HA, MYC, and fluorescent tags, such as green fluorescent protein, and multiples or combinations thereof.

In another example, the kit comprises a detection agent. In one example, the detection agent is capable of binding to one or more biomarkers. In one example, the detection agent is capable of binding to the monosaccharide-binding protein and/or the one or more O-glycosylated endoplasmic reticulum (ER)-resident proteins. In one example, the detection agent can be, but is not limited to, an enzyme-conjugated antibody, enzyme, or antibody that can produce and/or intensify a reaction. In one example, the enzyme can be horseradish peroxidase (HRP).

In another example, the kit comprises one or more standards. In one example, the kit comprises, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 standards.

As used herein, the term “standard” refers to a reference or a sample which is taken to be of a known value. In other words, the standard in an experiment is something which is used as a measure, norm, or model in comparative evaluations. For example, a positive control can be considered to be a standard. In another example, the standard can be the unmutated or wild-type form of a target (for example, a protein, a nucleic acid molecule and the like). In other examples, the term “standard” can also be used to refer to a protein ladder or a molecular weight reference used in gel electrophoresis to define substrate molecular weight. In yet another example, the term standard in the context of gene expression refers to the expression of a target gene in its unmodified environment. This unmodified environment can refer to, but is not limited to, the expression of the target gene in a disease-free subject. The standard can also be a representative value for gene expression of a specific gene obtained from a control group.

In one example, the standard in the kit as disclosed herein a biomarker as disclosed herein. In one example, the standard is a O-glycosylated endoplasmic reticulum (ER)-resident protein. In one example, the standards in the kit comprise one or more of the O-glycosylated endoplasmic reticulum (ER)-resident proteins as disclosed herein. In another example, the standards in the kit comprise, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 of the O-glycosylated endoplasmic reticulum (ER)-resident proteins. In another example, the standards in the kit comprise any one of the O-glycosylated endoplasmic reticulum (ER)-resident proteins as disclosed herein. In another example, the standards in the kit can be, but are not limited to of protein disulfide isomerase family A member 4 (PDIA4), calnexin (CANX), protein disulfide isomerase family A member 3 (PDIA3), Endoplasmic Reticulum Lectin 1 (ERLEC1) and heat shock 70 kDa protein 5 (glucose-regulated protein, 78 kDa) (HSPA5/GRP78/Bip) and combinations thereof, as disclosed herein.

The kit can be used to qualitatively assess or quantitatively measure the presence, amount, or functional activity of a target. In one example, the kit is used to determine the level of the one or more O-glycosylated endoplasmic reticulum (ER)-resident proteins in a sample according to the methods as disclosed herein; and/or compare the level of the one or more O-glycosylated endoplasmic reticulum (ER)-resident proteins according to the method as disclosed herein to a baseline level provided by the standard.

The kit can be an analytical tool. In one example, the kit can be an analytical biochemistry assay. In another example, the kit is an enzyme-linked immunosorbent assay (ELISA).

In one example, the kit is an ELISA kit that comprises a microwell plate; a sample diluent; a wash buffer; a substrate solution that can be detected using the detection agent; and a stop solution that can react with the substrate solution and allow visualisation.

As person skilled in the art would appreciate, the components of the kit or the kit can be adapted to use in accordance with the method as disclosed herein. The components of the kit or the kit be configured to be mixed as required by the methods disclosed herein. For example, the components disclosed herein can be mixed accordingly in a reaction vessel in order to obtain the information required according to the method disclosed herein. For example, in relation to an ELISA kit, a person skilled in the art would appreciate that an ELISA kit requires binding to the target analyte or biomarker or marker to the reaction vessel, detection of the marker with the required substrates, washing the reaction vessel and then subsequently detecting the presence, absence or level of the marker using a detection substrate.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

As used in this application, the singular form “a,” “an,” and “the” include plural references unless the context clearly dictates otherwise. For example, the term “a genetic marker” includes a plurality of genetic markers, including mixtures and combinations thereof.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and sub-generic groupings falling within the generic disclosure also form part of the disclosure. This includes the generic description of the embodiments with a proviso or negative limitation removing any subject matter from the genus, regardless of whether or not the excised material is specifically recited herein.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

### Example 1

### GALNTs Relocate to the ER in Malignant Liver Tumours

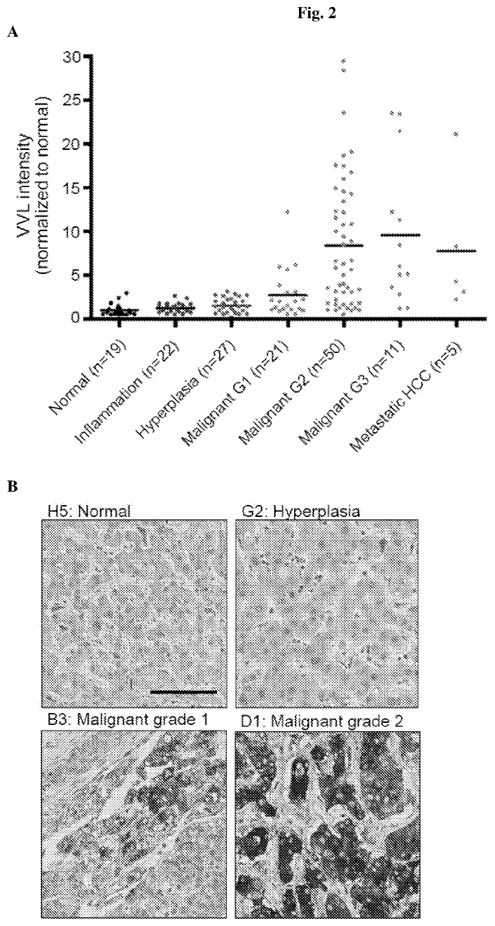

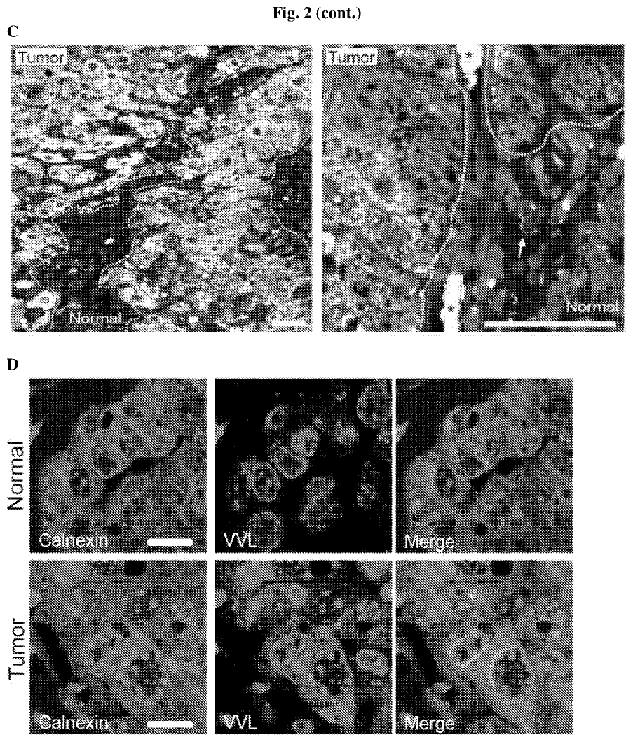

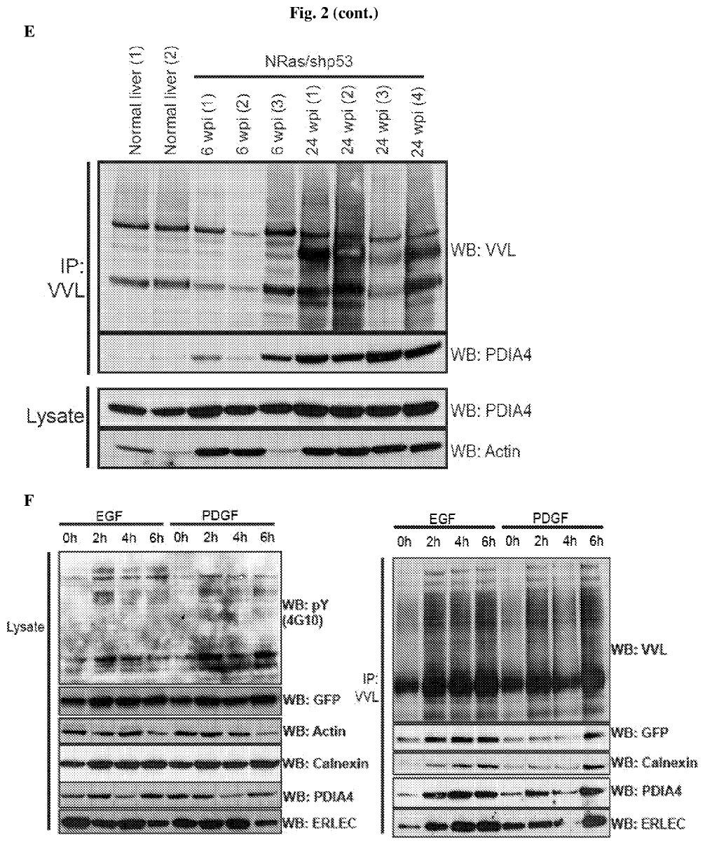

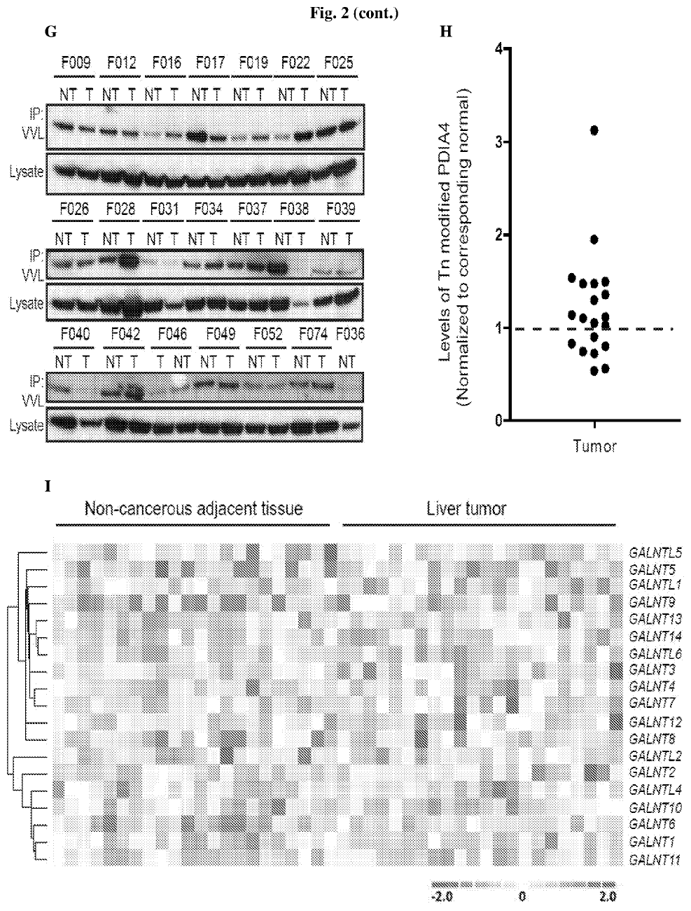

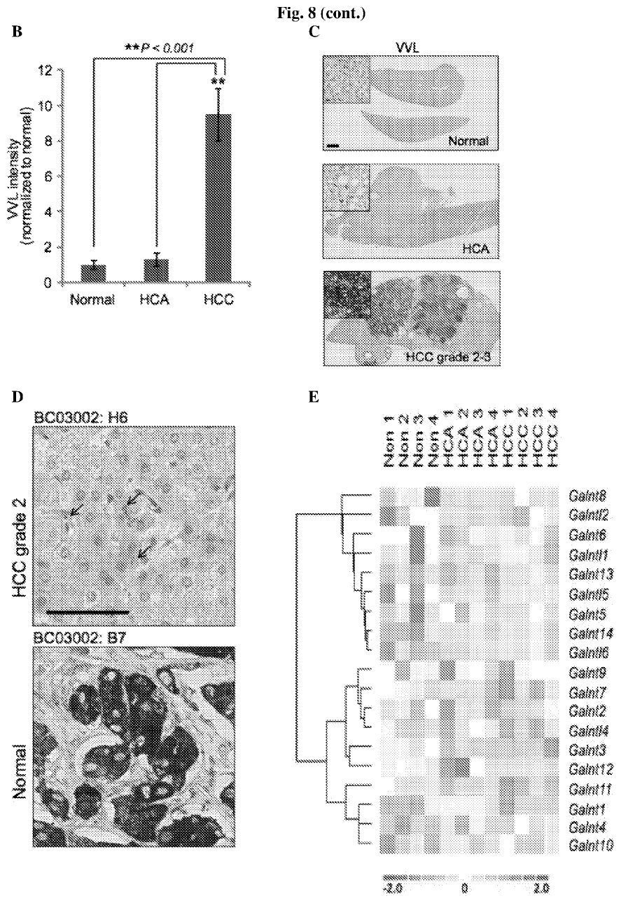

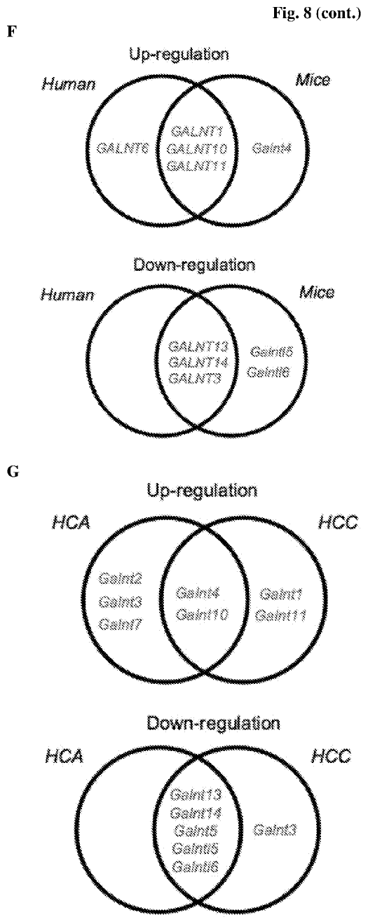

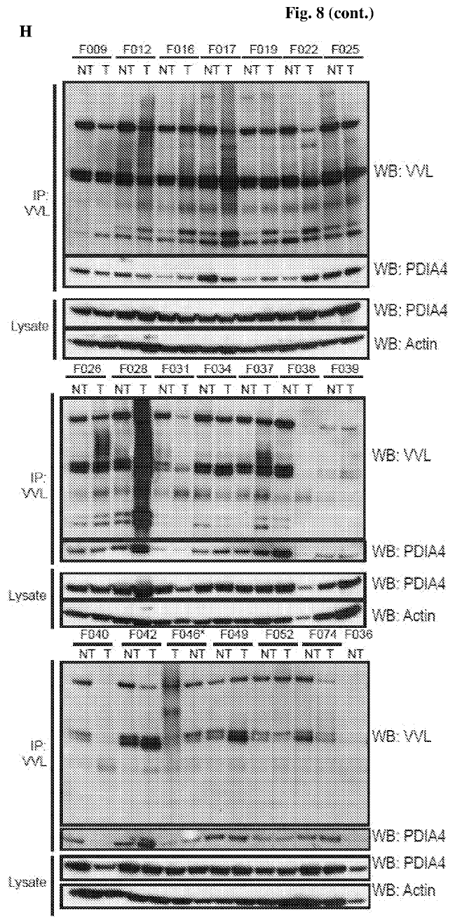

To quantify Tn levels in human liver cancer, 160 biopsies were stained with Vicia villosa lectin (VVL) (FIG. 8A). The biopsy cores originated from normal liver, hepatitis (In) and hyperplastic livers, hepatocellular carcinomas (HCC), and intra-hepatic cholangiocarcinoma of grades 1 to 3, as defined by a pathologist. Vicia villosa lectin (VVL) staining intensity was low in normal liver samples but elevated in 42/44 and 11/11 of carcinoma grades 2 and 3, respectively (FIGS. 2A and 2B). The median Tn increase in grade 3 carcinomas was approximately 4-fold over control tissues, with up to a 10-fold increase. No correlation with tumour type could be detected.

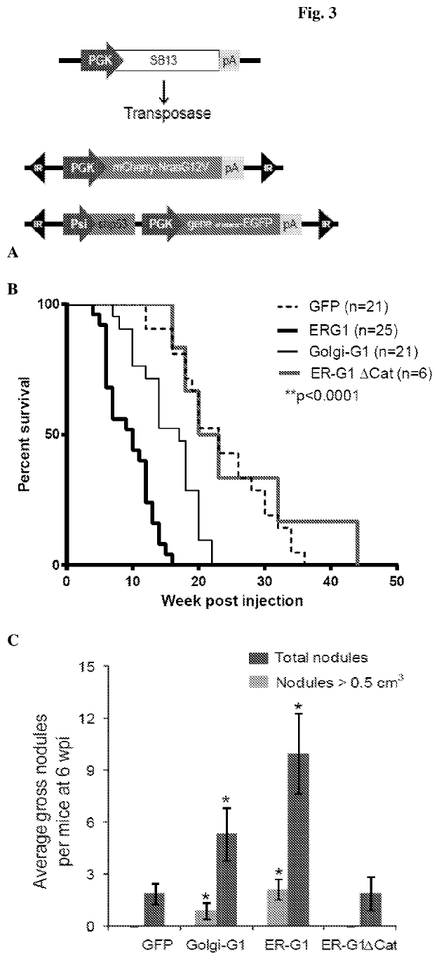

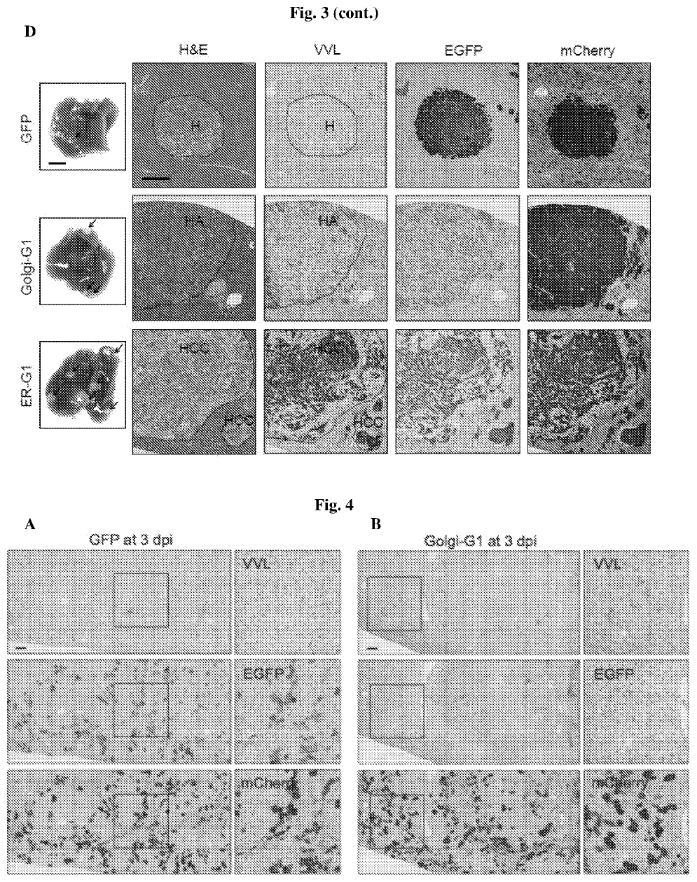

Next, liver tumours were induced in mice using hydrodynamic injection of plasmids encoding for Sleeping Beauty transposase and NRas-G12V and anti-p53 shRNA (shp53) (FIG. 3A). At 20 weeks post-injection (wpi), the mice were euthanized and livers dissected. Approximately 40% of mice showed hepatocellular adenomas (HCA) and 60% showed hepatocellular carcinoma (HCC). While most of the cells in the hepatocellular adenoma (HCA) had low Tn levels, in hepatocellular carcinoma (HCC), most cells had elevated Vicia villosa lectin (VVL) staining (FIGS. 8B and 8C). Overall, the patterns were similar between mouse and human tumours, with an increase in Tn intensity and frequency correlating with tumour severity. This shows that the plasmids encoding for Sleeping Beauty transposase and NRas-G12V and anti-p53 shRNA (shp53) (FIG. 3A) can induce hepatocellular adenomas (HCA) and hepatocellular carcinoma (HCC), which can be differentiated by levels of Tn using Vicia villosa lectin (VVL) staining.

On high-magnification immunofluorescence images of human and mouse samples, cells with low levels of staining were observed to show positive Tn expression in the small peri-nuclear structures consistent with the Golgi (FIGS. 2C and 8D), whereas, in cells with high Tn levels, the staining was distributed within the whole cell (FIG. 8D), with a reticulated appearance (FIG. 2C). Indeed, co-staining with the ER marker Calnexin indicated that Tn localizes with Calnexin in advanced tumours (FIG. 2D).

Protein disulfide isomerase 4A (PDIA4) has been proposed to be a GALNT substrate, and because it is an ER-resident protein, its level of glycosylation is expected to be low in normal conditions but elevated upon GALA. Using immunoprecipitation of N-acetylgalactosamine (GalNAc)-modified proteins, PDIA4 glycosylation was observed to be very low in normal liver samples but increases in early tumours and is markedly increased in late-stage tumours (FIG. 2E). To verify that GALA is also activated in human cancer tissues, 20 samples of paired non-tumour and tumour tissue from recently resected livers were analysed (Table 2). In several non-tumour tissues, level of PDIA4 glycosylation were very low, comparable to normal mouse liver. By comparison, nearly all tumour tissues had elevated or very elevated levels of PDIA4 glycosylation (FIG. 2G, 2H, 8H). Some non-tumour tissues also displayed elevated PDIA4, possibly reflecting that these livers are often in a diseased state. When compared pair-wise, about 65% of tumour tissues had higher levels of ER O-glycosylation than the non-tumoural tissue.

Together, these results establish that GALNTs are relocated to the ER in liver tumours, and suggest that GALA drives the observed increase in Tn levels.

### Example 2

### Liver Tumours Express High Levels of GALNT1

To explore which GALNTs are involved in hepatocellular carcinoma (HCC), the expression levels of all family members in paired normal (N) and diseased (T) samples from 22 patients with hepatocellular carcinoma (HCC) were analysed (FIG. 21). In parallel, 12 mouse samples were analysed, with three groups of normal, hepatocellular adenoma (HCA), and hepatocellular carcinoma (HCC), as determined by histopathological assessment (FIG. 8E). Interestingly, relatively few GALNTs were up-regulated: GALNT1, -T10 and -T11 were up in both human and mouse, whereas GALNT4 and -T6 appeared to be specific for mouse and human tumours, respectively (FIGS. 8F and 8G). Conversely GALNT3, -T13 and -T14 were downregulated in both species (FIGS. 8F and 8G). These data suggest that GALNT isoforms do not all have the same effect on tumour development. Furthermore, isoforms have also differences in substrate specificity, with GALNT1 a primary enzyme in normal liver tissues. These results substantiate a role for GALNT1 in hepatocellular carcinoma (HCC).

### Example 3

### An ER-Targeted GALNT1 Doubles the Speed of Tumour Development

To directly test the effect of ER relocation of GALNT1, a chimeric form of the enzyme that is constitutively targeted to the ER was generated. In the tumour induction system, mice were injected with plasmids encoding GFP or GFP-tagged forms of wild-type Galnt1 (Golgi-G1), ER-localized Galnt1 (ER-G1), or ER-G1 catalytically inactive (ER-G1ΔCat) (FIG. 3A). After injection, mouse survival was monitored: the median survival was 23 weeks post injection (wpi) for control (GFP) mice and ER-G1ΔCat mice; 10 weeks post injection (wpi) for ER-G1 mice (FIG. 3B); and 17 weeks post injection (wpi) for Golgi-G1 (WT) mice. This indicates that ER-localized Galnt1 (ER-G1) leads to higher mortality.

At 6 weeks post injection (wpi), about 40% of ER-G1 mice were dying, whereas no mortality was observed in controls. At this stage, 9 mice were sacrificed from each group for assessment. Striking differences were noted in the livers, with 5-fold more nodules and larger tumours in the ER-G1 group (FIG. 3C) as compared with the control group. The Golgi-G1 mice displayed an intermediate abundance of nodule formation. All tumours examined were positive for GFP and mCherry expression, indicating successful co-expression of the transgenes. In the control mice, 9/9 tumours showed low Tn levels, whereas, in the ER-G1 group, all tumours displayed high Tn levels; Golgi-G1 tumours showed variable, intermediate levels (FIG. 3D). Tumours from mice in the control group presented clear margins, whereas ER-G1 mice showed multifocal tumours and tumour cells interdigitated within the normal tissue (FIG. 3D).

### Example 4

ER-G1 Promotes Tumour Growth from an Early Stage

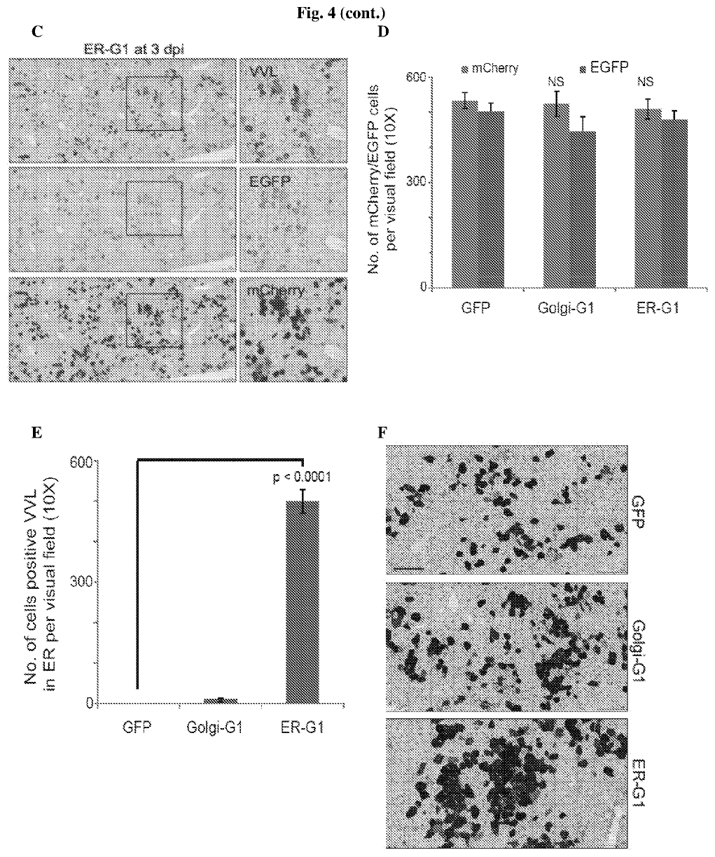

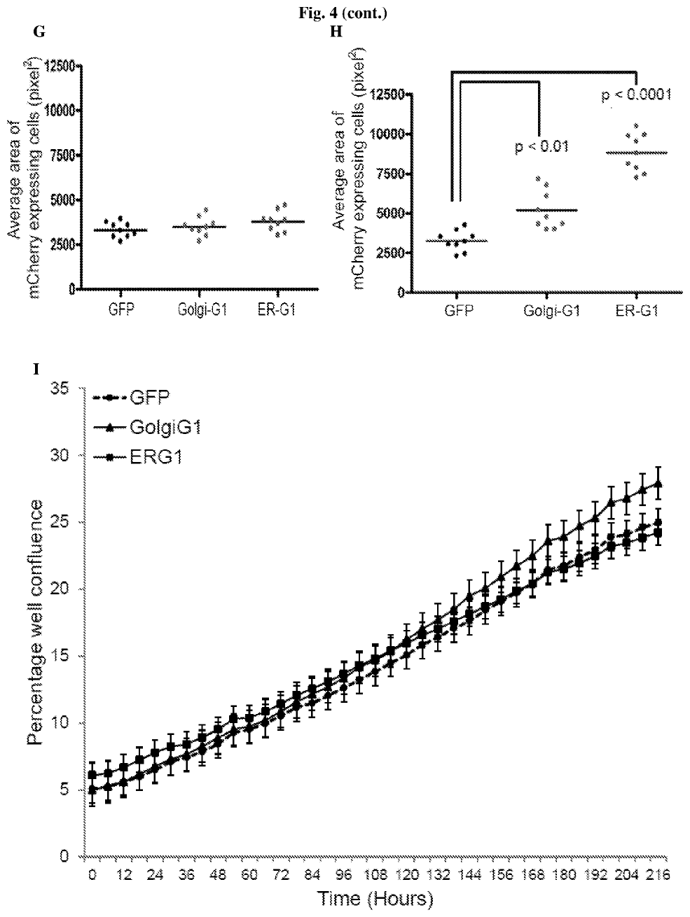

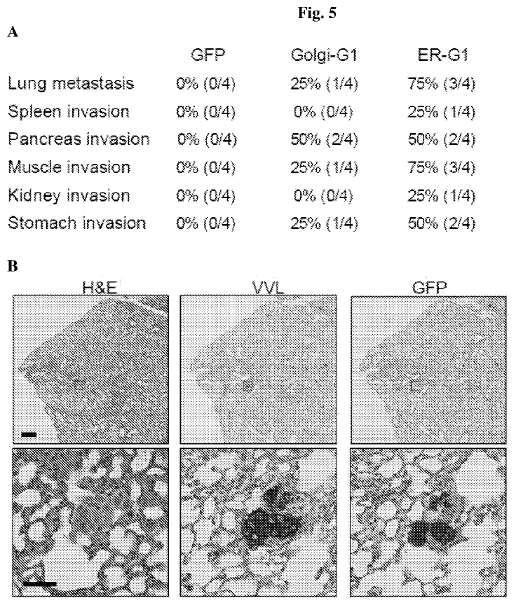

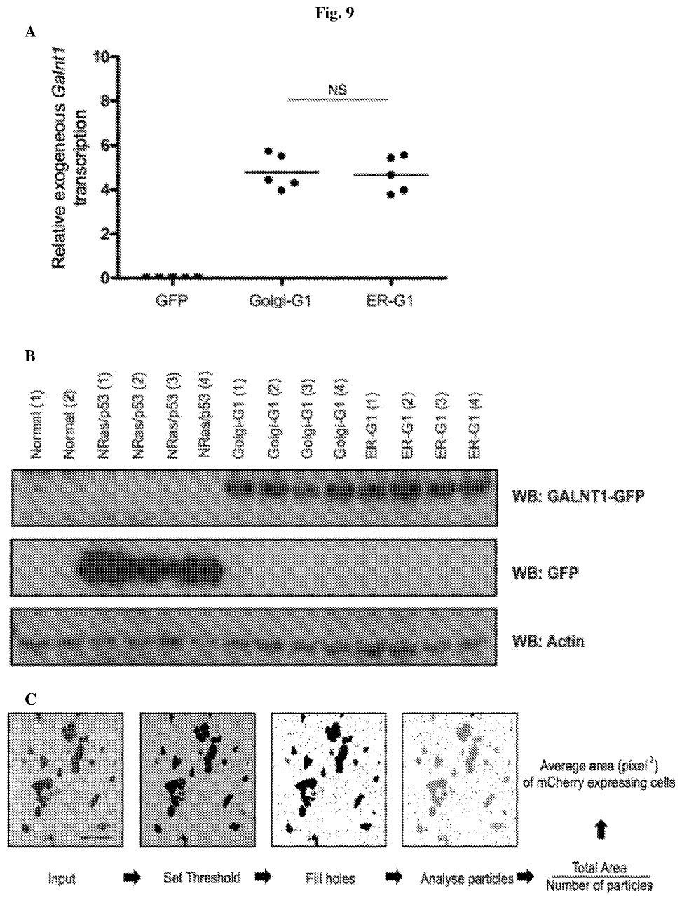

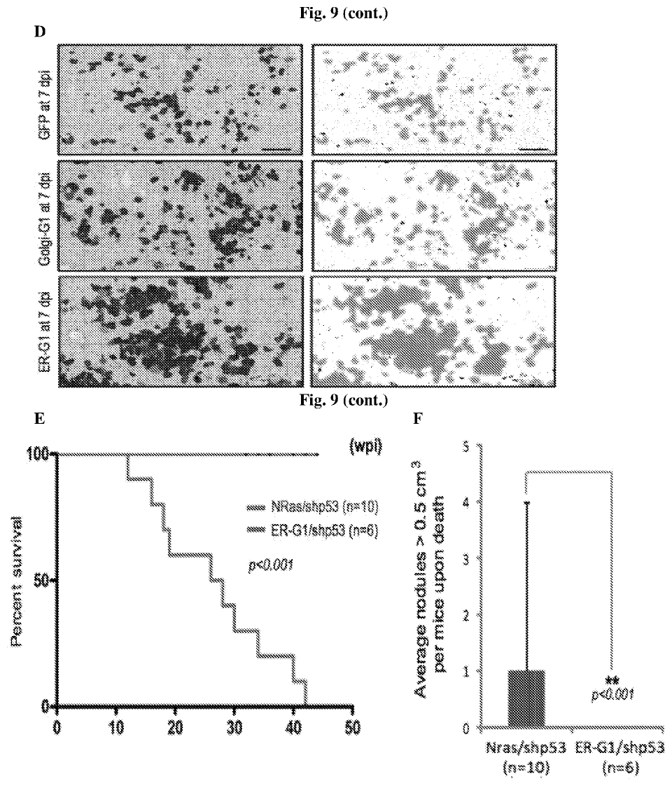

To explore how ER-G1 stimulates tumour growth, it was investigated when its effects become detectable. Transfected liver cells were observed to be clearly detectable by GFP and mCherry labelling as early as 3 days post-injection (dpi), with a consistent number of transfected cells among the mice for all conditions (FIG. 4A-4D). The levels of expression of the GALNT1 constructs were verified to be similar at the transcript and protein levels (FIGS. 9A and 9B).

Unlike in the other conditions, ER-G1-transfected cells were strongly labelled with Vicia villosa lectin (VVL), indicating that expression of the construct reproduced the high Tn levels observed in advanced natural tumours (FIGS. 4C and 4E). At 3 days post-injection (dpi), most GFP/mCherry-positive cells appeared as single, isolated cells, suggesting very little cell division. In mice injected with the control plasmids, this appearance did not change significantly at 7 days post-injection (dpi), suggesting that the initial average doubling time of transformed cells is at least 6 days. By comparison, clusters of transfected cells were observed in ER-G1-expressing cells, indicative of cell division and small nodule formation. The size of these nodules were quantified by measuring their average area (FIGS. 4F, 4G, 4H, 9C and 9D), and measured a 4-fold increase in size in ER-G1-expressing conditions as early as 7 days post-injection (dpi) (FIGS. 4F, 4G and 4H). Golgi-G1-expressing cells displayed an intermediate phenotype throughout (FIGS. 4F, 4G and 4H).

### Example 5

ER-G1 Expression does not Induce Tumour Formation Nor does it Promote Proliferation In Vitro

To test if ER-G1 functions as an oncogene like NRas-G12V, 6 mice were injected with ER-G1 and shp53 along with a control group (NRas/shp53). Histological analysis confirmed that a similar proportion of cells were transfected in both conditions. However, the ER-G1/shp53 mice did not experience any lethality, unlike the mice in the NRas/shp53 group (FIG. 9E). At 40 weeks post injection (wpi), the livers from the ER-G1/shp53 mice did not display any visible nodules (FIG. 9F). Yet, a significant number of Tn-positive cells were detected in these livers, indicating that the expression of ER-G1, in the absence of NRas, is not toxic but does not induce cell growth or proliferation (FIG. 9G).

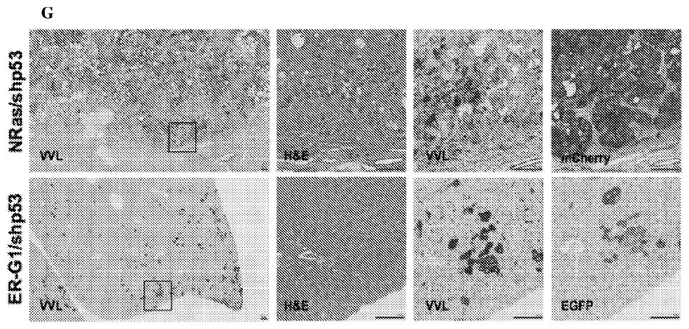

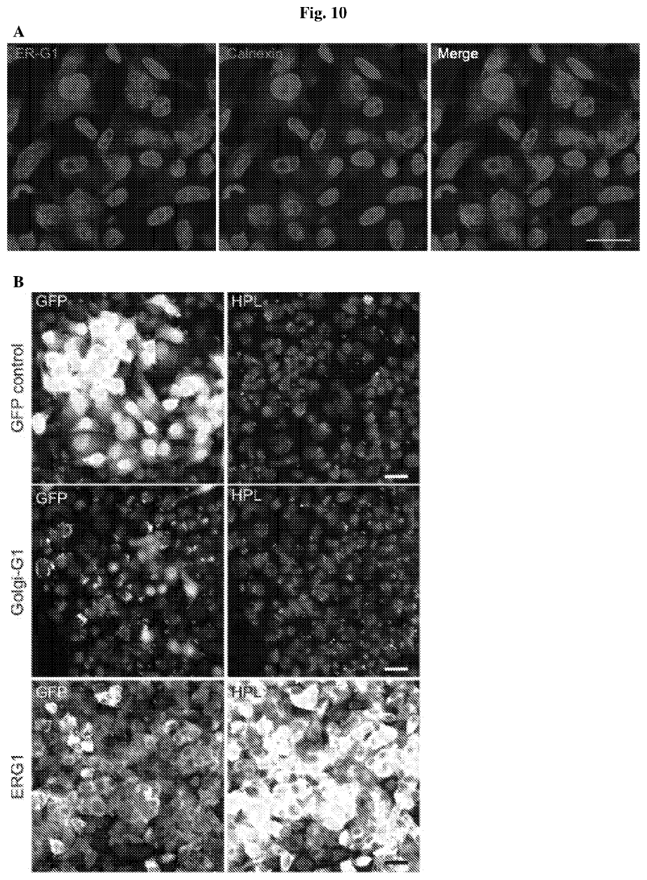

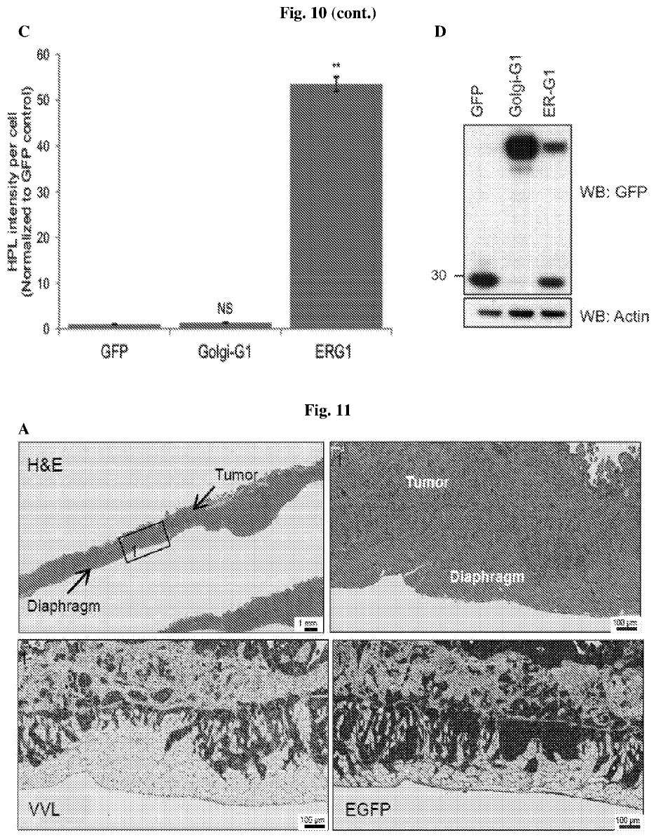

To test if ER-G1 promotes proliferation in vitro, a series of stable cell lines expressing GFP, Golgi-G1, or ER-G1 in hepatocellular carcinoma (HCC)-derived HepG2 cells were derived. The subcellular localization of the constructs was verified, and in particular that ER-G1 co-localizes with the ER marker Calnexin (FIG. 10A). ER-G1 HepG2 cells had a 50-fold increase in Tn staining levels as compared with both GFP and Golgi-G1 cells, with Golgi-G1 overexpression having no significant effect (FIGS. 10B and 10C). Yet, levels of expression of the enzymes were similar, Golgi-G1 levels even being slightly higher (FIG. 10D).

Average doubling time was observed to be similar for all cell lines and revolved around 24 hours (FIG. 41). Thus, ER-G1 does not stimulate proliferation in vitro and is unable to induce tumour formation in vivo, yet stimulate tumour growth from a very early stage. This suggests that ER-G1 stimulates cancer cell proliferation by enabling tumour expansion rather than directly stimulating cell growth and division.

### Example 6

### ER-G1 Promotes Tissue Invasion, Metastases and ECM Degradation

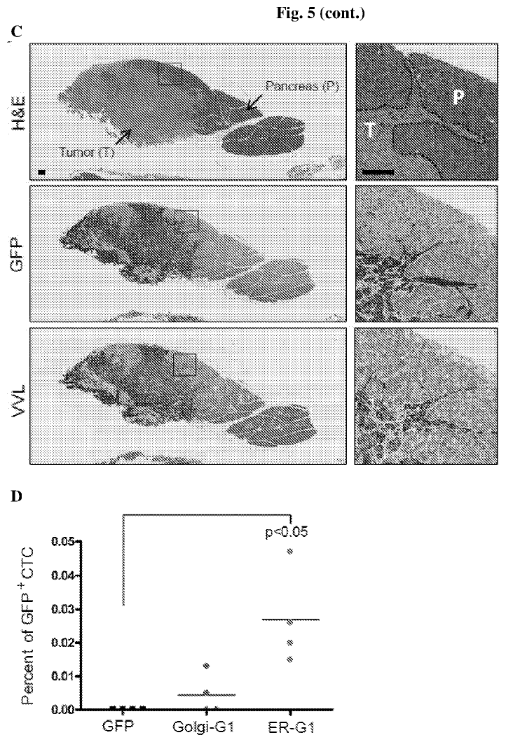

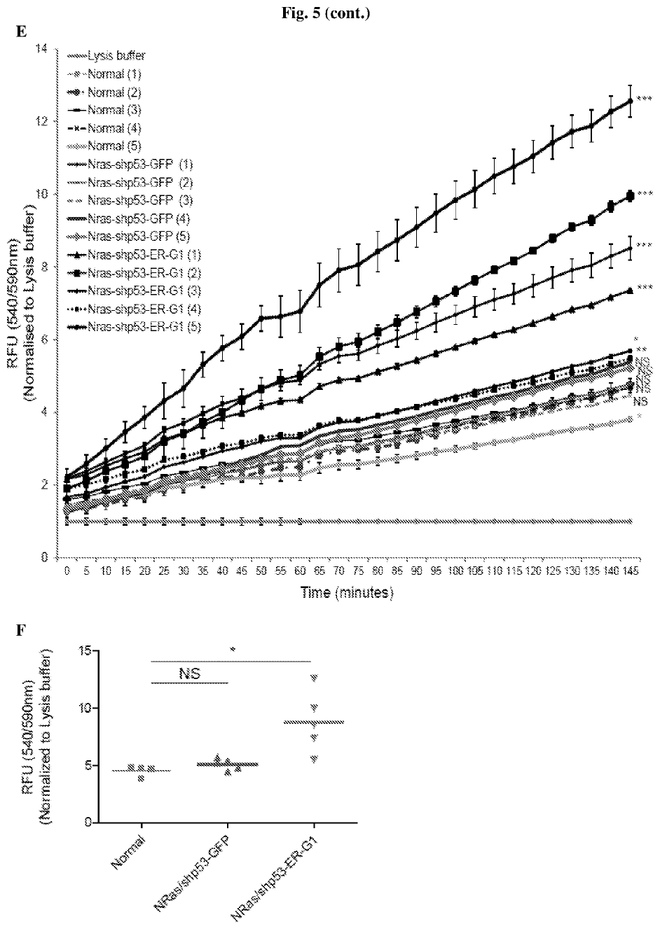

Based on tumour morphology, ER-G1 was hypothesised to stimulate tumour growth by facilitating tissue remodelling and invasion. Consistently, in ER-G1-injected mice that died at 16 weeks post injection (wpi), metastases were observed in various organs, particularly in the lungs (FIG. 5A-5C). Control mice showed no metastases, with rare occurrences in Golgi-G1 mice (FIG. 5A).

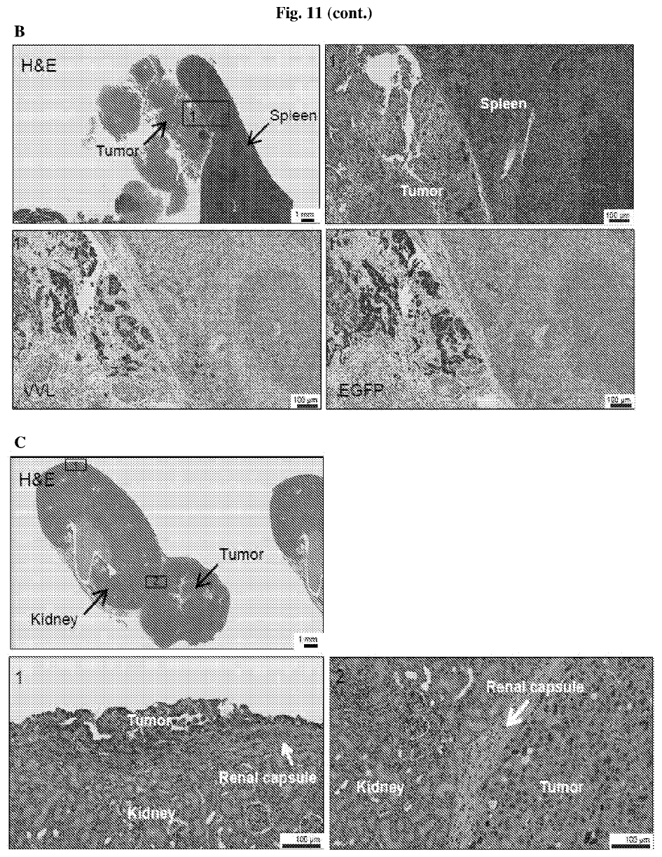

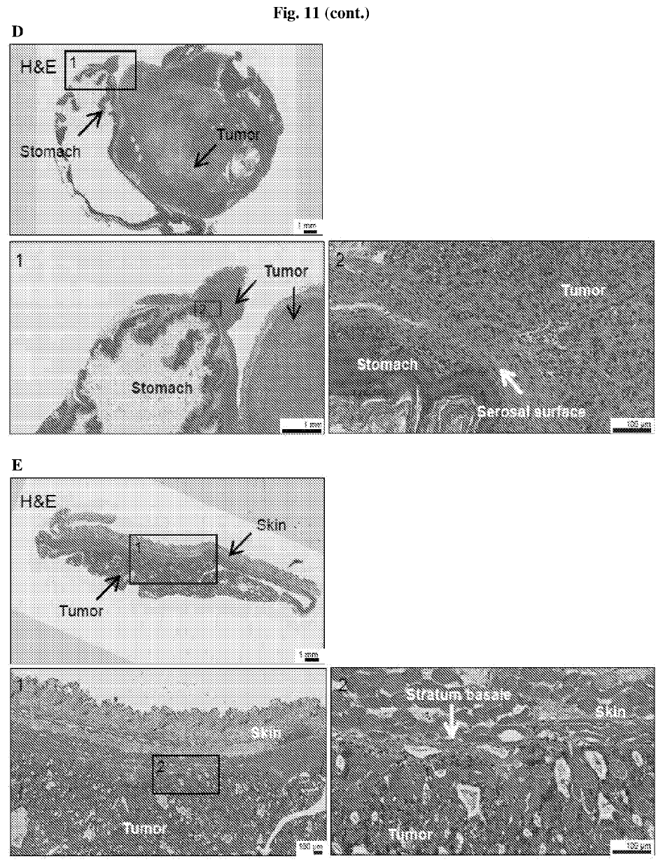

Circulating tumour cells (CTCs) reveal a high capacity of cancer to escape the primary environment. Significant levels of CTCs were found in 3/4 ER-G1 mice at the time of sacrifice, but none detectable in the control mice at the same stage (FIG. 5D). Upon dissection, tumours were observed to frequently adhere to and invade neighbouring organs, such as the pancreas (FIG. 5C), diaphragm, spleen, kidney, stomach, skin, and peritoneal muscles (FIG. 11A-11E). This phenotype of highly aggressive tumours was virtually absent in the control and Golgi-G1 groups.

### Example 7

### ER-G1 Induces ECM Degradation

Results to this point suggested that ER-G1 tumour cells were more effective at invading surrounding tissues and breaking free from their tissue of origin, which is often dependent on the capacity to cleave ECM components and thus express matrix proteases. Consistently, 5/5 ER-G1 tumour lysates displayed significantly higher levels of matrix metalloproteinases (MMP) activity than normal liver and 4/5 higher than early-stage control tumours (FIGS. 5E and 5F).

Whereas cancer cells tend to have higher matrix metalloproteinases (MMP) activity in general, studies have suggested that macrophages or cancer-associated fibroblasts play a key role in matrix degradation. To test if ER-G1 could stimulate matrix degradation in a cell-autonomous fashion, matrix metalloproteinases (MMP) activity was tested in stable HepG2 cell lines. ER-G1 cells displayed higher matrix metalloproteinases (MMP) activity (FIG. 5G), and, when the cells were seeded on fluorescently labelled gelatin sheets, the ER-G1 cells showed a 6-fold increase in degradation (FIGS. 5H and 5I).

### Example 8

### ER Localization of GALNT1 Induces Matrix Metalloproteinase-14 (MMP14) Hyper-Glycosylation

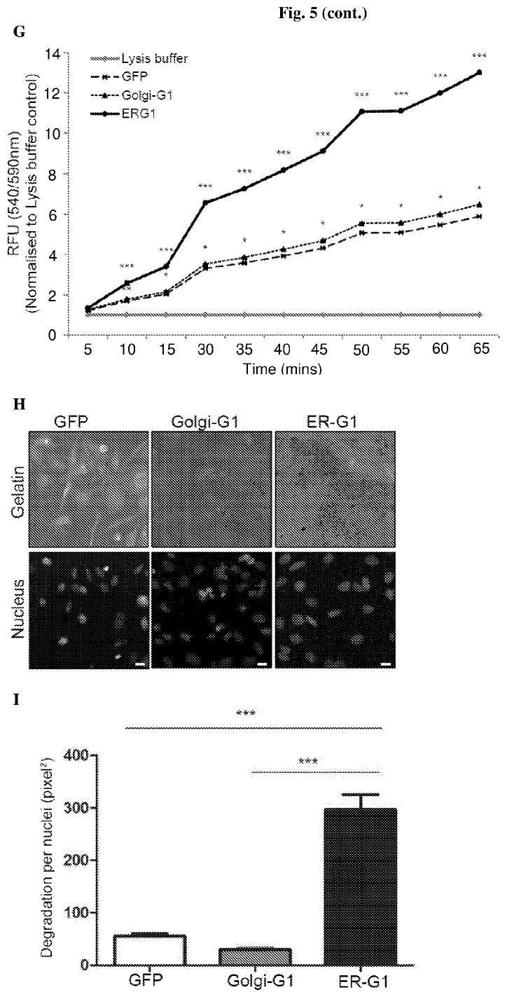

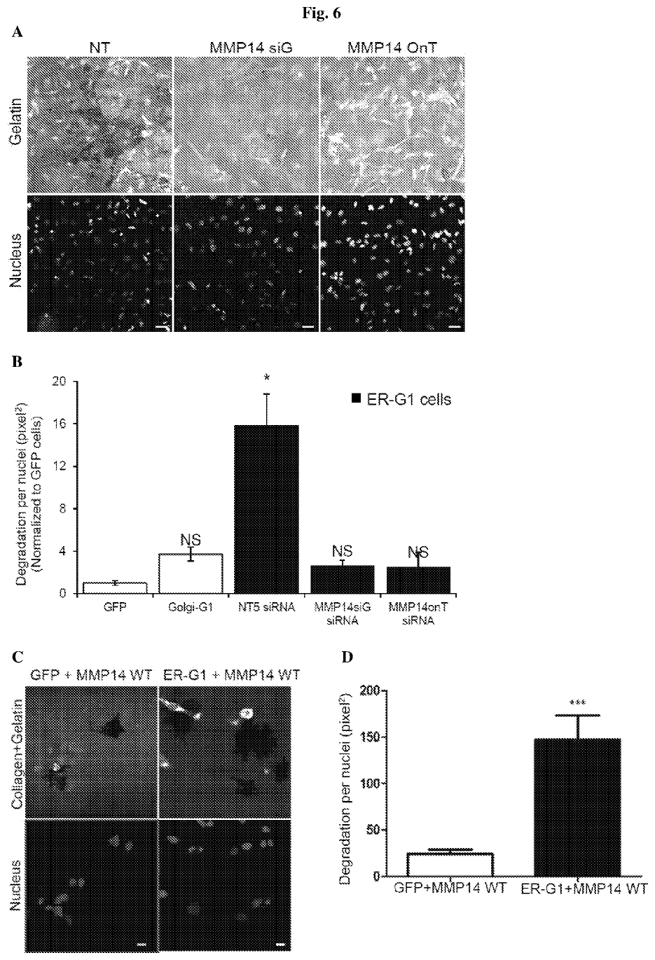

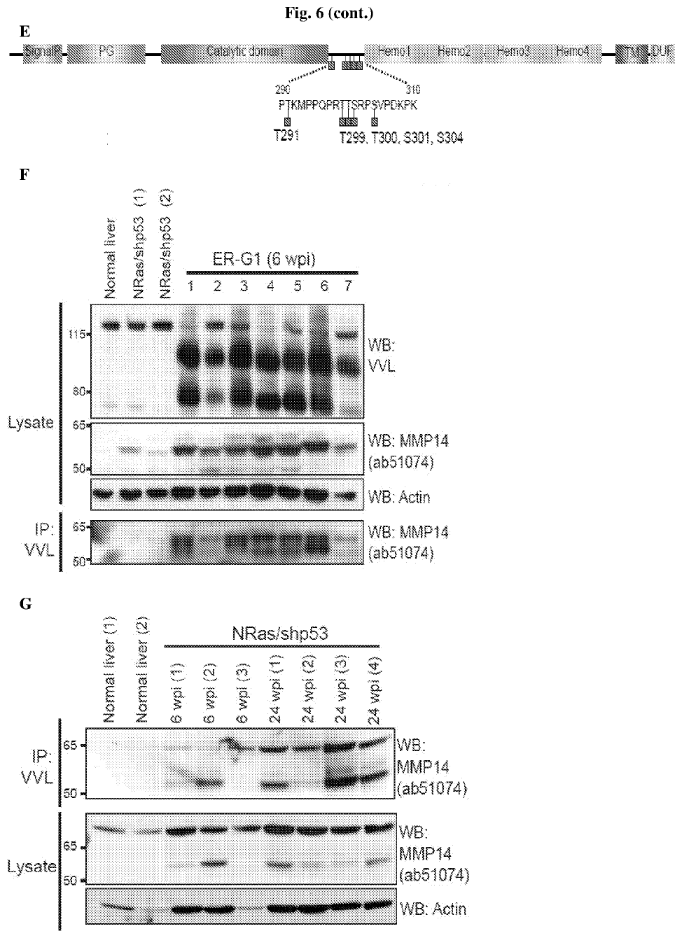

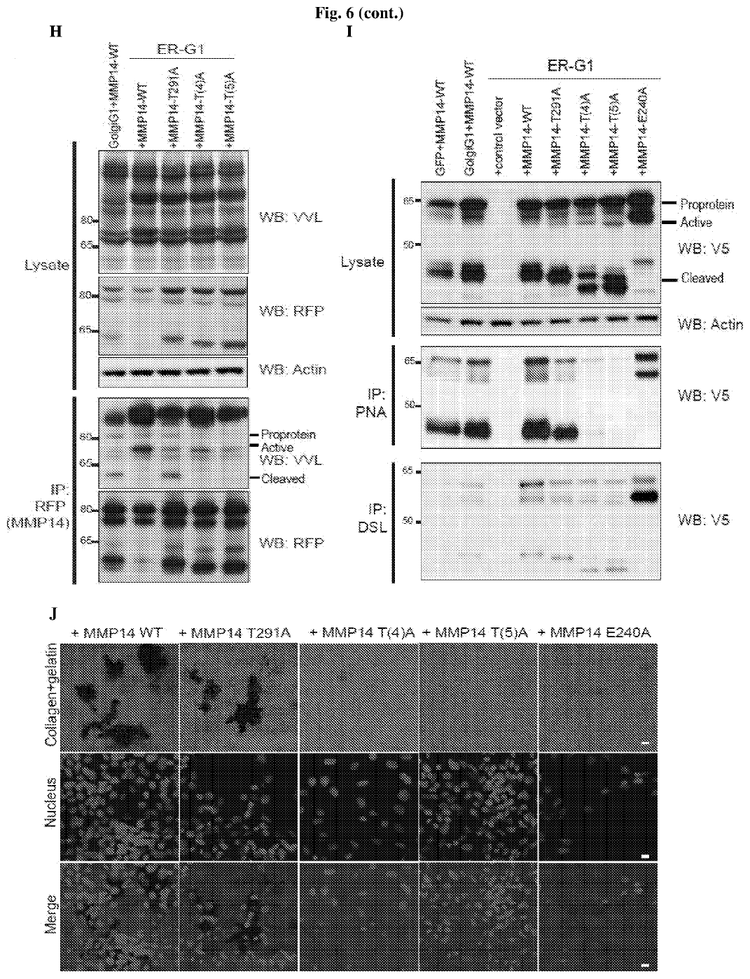

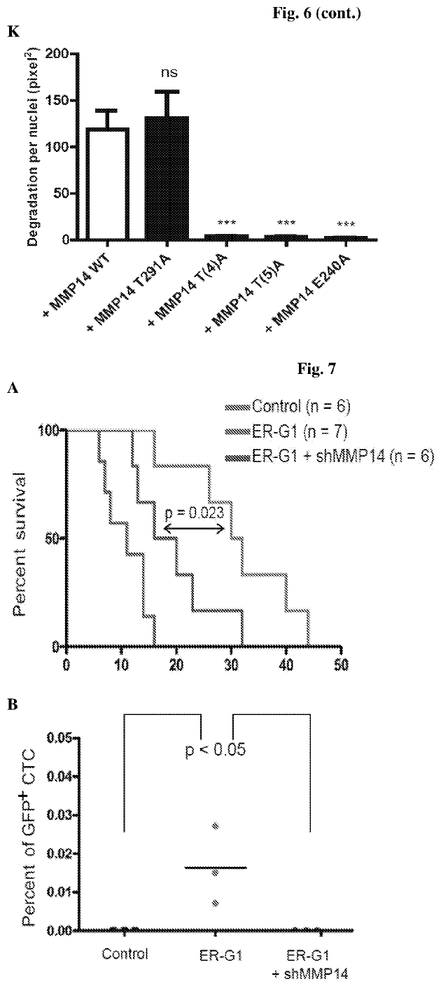

Numerous studies point to the membrane-tethered membrane type 1 matrix metalloproteinase (MT1-MMP, alias matrix metalloproteinase-14 (MMP14)) as a key enzyme for localized ECM breakdown. To test the importance of matrix metalloproteinase-14 (MMP14), two different small interfering ribonucleic acid (siRNA) pools were selected for their ability to deplete the protein (FIG. 12A). These small interfering ribonucleic acid (siRNA) pools abrogated the ER-G1 induced increase in gelatin degradation, suggesting that matrix metalloproteinase-14 (MMP14) is mediating ER-G1 effect (FIGS. 6A and 6B).

Next, matrix metalloproteinase-14 (MMP14) was overexpressed in the HepG2 cell lines. These cell lines rapidly digested a gelatin film. Native collagen, in a triple helix form, is more resistant to proteolysis than the denatured collagen found in gelatin. When a layer of collagen I was added on top of the gelatin, degradation was slower, and the MMP14-transfected ER-G1 cells (ER-G1+MMP14 WT) were significantly more active than the MMP14-transfected control cells (GFP+MMP14 WT) (FIGS. 6C and 6D). Overall, these data suggest that ER-G1 expression up-regulates matrix metalloproteinase-14 (MMP14) activity.

matrix metalloproteinase-14 (MMP14) is known to be O-glycosylated on five residues located in the hinge domain between residues T291 and S304, with glycosylation of S304 still debated (FIG. 6E). To test whether ER-G1 stimulates the glycosylation of matrix metalloproteinase-14 (MMP14), Tn modified proteins were pull down using Vicia villosa lectin (VVL) and probed for matrix metalloproteinase-14 (MMP14) from liver tumours derived from ER-G1 expressing tumours at 6 weeks post injection (wpi) (FIG. 6F). A similar approach was also used for control tumours at 6 and 24 weeks post injection (wpi) (FIG. 6G). While matrix metalloproteinase-14 (MMP14) protein levels were more elevated in ER-G1 tumours, the levels of Tn were significantly higher. Furthermore a higher level of glycosylation was also observed in control tumours at 24 weeks post injection (wpi) (FIG. 6G). To verify that these effects were cell autonomous, matrix metalloproteinase-14 (MMP14) glycosylation was tested in HepG2 derived cell lines. However, endogenous matrix metalloproteinase-14 (MMP14) levels were low and the Tn signal was very weak. A possible complication is that O-glycans on matrix metalloproteinase-14 (MMP14) are modified upon transit of the protein through the Golgi. Another approach was tested by re-deriving Golgi-G1 and ER-G1 cell lines in a background of HepG2 Cosmc^{-/-} cells. These cells are unable to extend the N-acetylgalactosamine (GalNAc) sugar with galactose and can only cap N-acetylgalactosamine (GalNAc) with sialic acid, which can be removed with a neuraminidase treatment. These HepG2 Cosmc^{-/-} cells were transfected with various mCherry-tagged matrix metalloproteinase-14 (MMP14), which was subsequently immuno-precipitated and probed for Tn. Under these conditions, matrix metalloproteinase-14 (MMP14) was markedly increased in ER-G1-expressing cells as compared with Golgi-G1 (FIG. 6H). The Tn signal was dependent on the cluster of residues in the hinge domain as mutants for this cluster showed reduced glycosylation (FIG. 6H) (see below for a description of the mutants).

Another way to evaluate glycosylation levels is to use metabolic labeling of O-glycans with an azide-modified analog of N-acetylgalactosamine (GalNAc), called GalNAz. Once incorporated into glycoproteins, the residue can be modified by click chemistry and conjugated to a FLAG peptide. It was observed that GalNAz incorporation was increased by 3.5-fold in ER-G1-expressing HepG2 cell lines compared with GFP cells (FIGS. 12B and 12C).

Finally, extended O-glycans can be detected in part with lectins, such as peanut agglutinin (PNA) and Datura stramonium Lectin (DSL) (FIG. 12D). With both lectins, an increase in reactivity of matrix metalloproteinase-14 (MMP14) was observed in ER-G1-expressing cells as compared with control and Golgi-G1-expressing cells (FIG. 6I).

Overall, these approaches indicate that localization of GALNT1 in the ER leads to an increase in glycosylation of matrix metalloproteinase-14 (MMP14) between 2.5- and 8-fold depending on the technique used.

### Example 9

### Matrix Metalloproteinase-14 (MMP14) Glycosylation is Essential for ECM Degradation

To verify that increased glycosylation of matrix metalloproteinase-14 (MMP14) occurs on the residues previously identified, three mutant forms of matrix metalloproteinase-14 (MMP14) were generated: a single-point mutant, T291A; a mutant bearing four alanine substitutions, T299A-T300A-S301A-S304A (T(4)A); and one bearing five alanine substitutions, T291A-T299A-T300A-S301A-S304A (T(5)A). These glycosylation mutants displayed an expected reduction in lectin binding (FIGS. 6H and 6I).

Glycosylation in the hinge region has been proposed to affect matrix metalloproteinase-14 (MMP14) maturation and stability. However, the mutants did not display massive changes in matrix metalloproteinase-14 (MMP14) expression levels (FIGS. 6H, 6I and 12F). Both pro-protein and active forms were relatively constant in the HepG2 cell lines, suggesting that glycosylation does not affect matrix metalloproteinase-14 (MMP14) stability in these cells (FIGS. 12E and 12F).

Matrix metalloproteinase-14 (MMP14) is known to self-cleave, generating a 44-kDa form. In contrast with a catalytically inactive form of matrix metalloproteinase-14 (MMP14) (MMP14-E240A), the glycosylation mutants displayed this short form, indicating activity in self-proteolysis, consistent with previous reports (FIGS. 6H, 6I and 12F). However, when tested in the cell-based ECM degradation assay, MMP14-T(4)A and MMP14-T(5)A mutants had a complete loss of activity, comparable with that of an E240A mutant (FIGS. 6J and 6K). Thus matrix metalloproteinase-14 (MMP14) glycosylation is essential for ECM degradation.

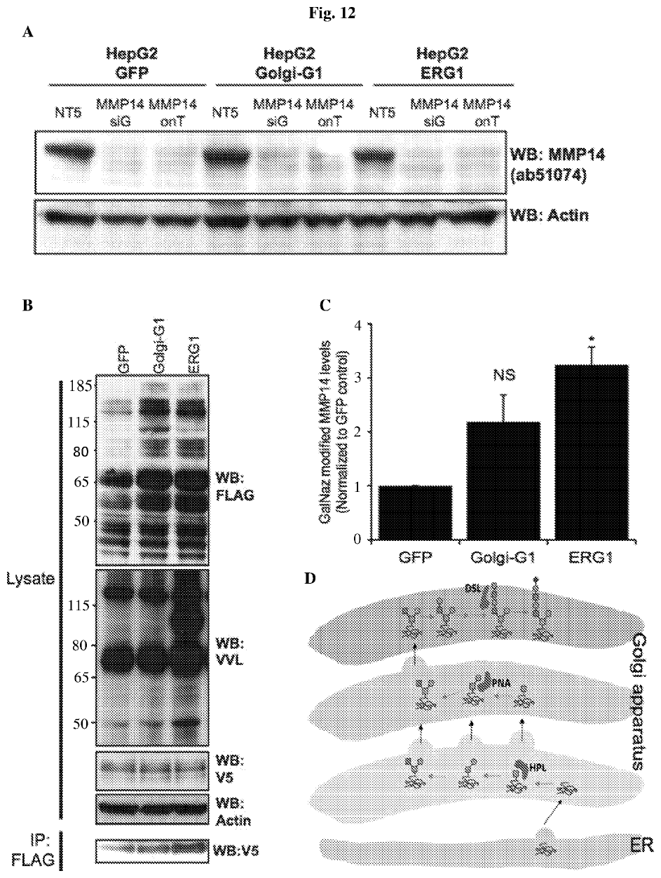

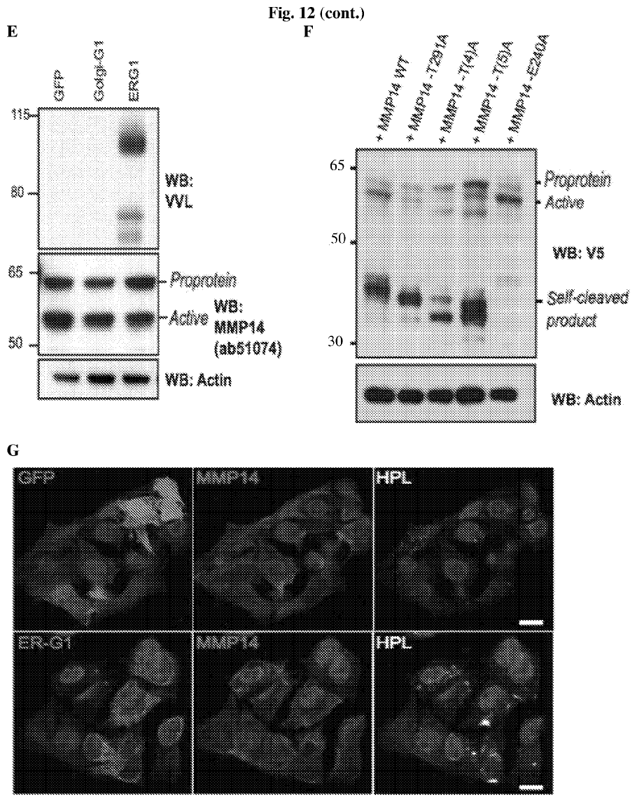

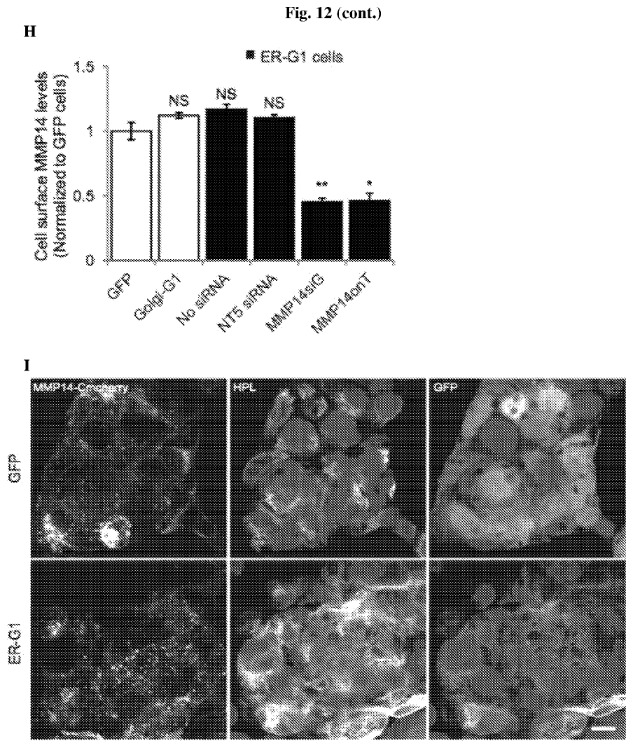

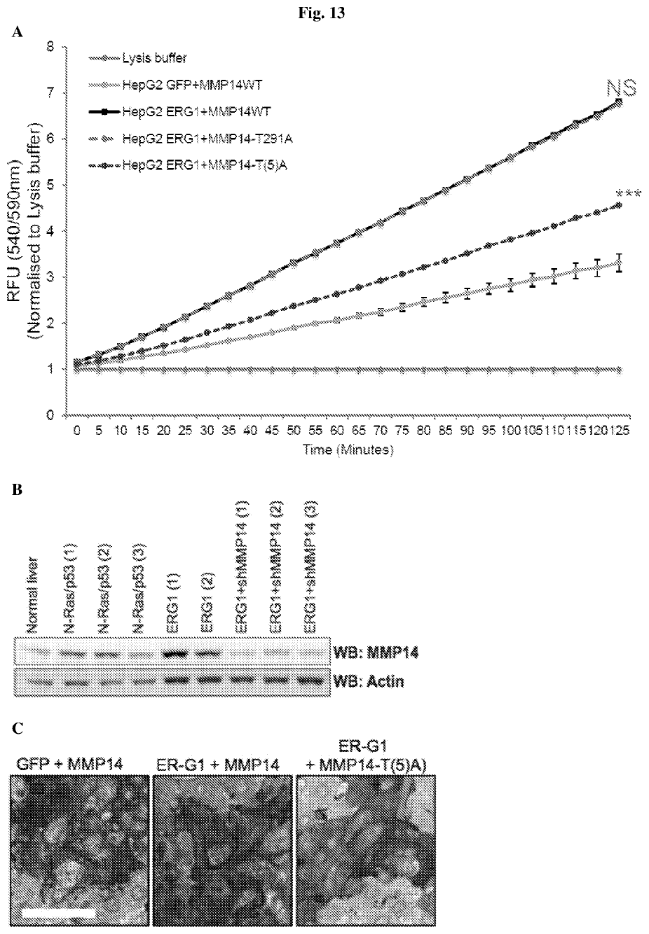

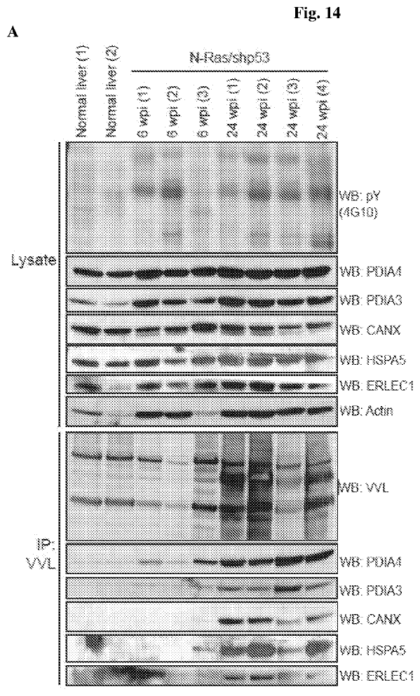

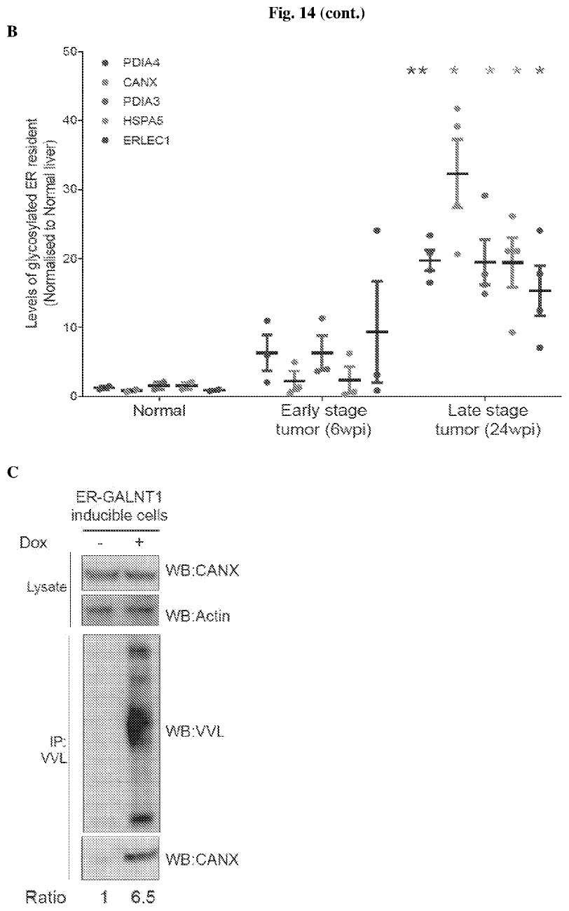

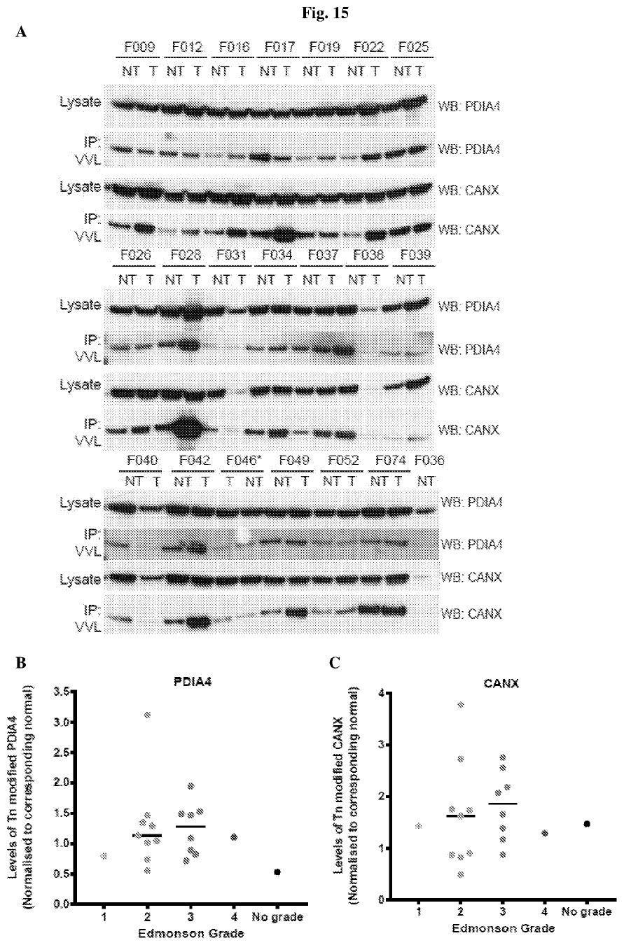

Cell-surface exposure of endogenous matrix metalloproteinase-14 (MMP14) was measured in the three HepG2 cell lines by quantitative immunofluorescence using non-permeabilized cells (FIGS. 12G and 12H). While matrix metalloproteinase-14 (MMP14) small interfering ribonucleic acid (siRNA) depletion clearly reduced the signal, there was no significant effect of ER-G1-expressing cells, suggesting that glycosylation is not required for trafficking to the cell surface (FIGS. 12G and 12H). Further analysis of matrix metalloproteinase-14 (MMP14) intracellular distribution did not reveal a major change in intracellular distribution of matrix metalloproteinase-14 (MMP14) (FIG. 12I). Together, these data suggest that glycosylation does not affect trafficking or cell surface stability of matrix metalloproteinase-14 (MMP14). By contrast, measurement of matrix metalloproteinases (MMP) activity using the fluorescent substrate assay suggested a direct effect of glycosylation on matrix metalloproteinase-14 (MMP14) activity (FIG. 13A). Indeed, the T(5)A mutant showed a strong reduction of activity compared to wild-type matrix metalloproteinase-14 (MMP14) (FIG. 13A).

### Example 10

Matrix Metalloproteinase-14 (MMP14) Glycosylation Promotes Tumour Growth from an Early Stage

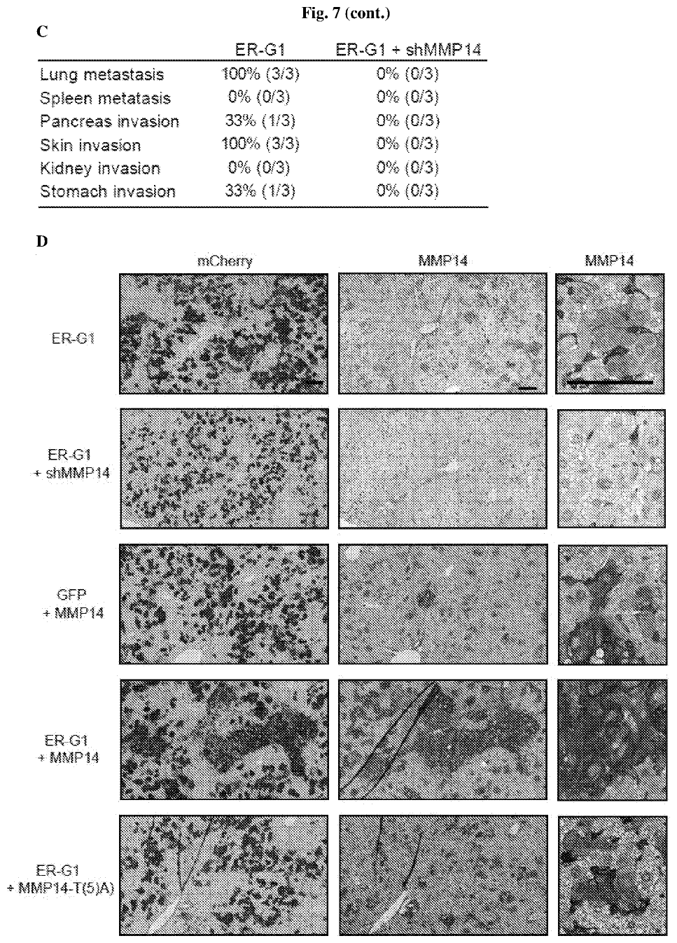

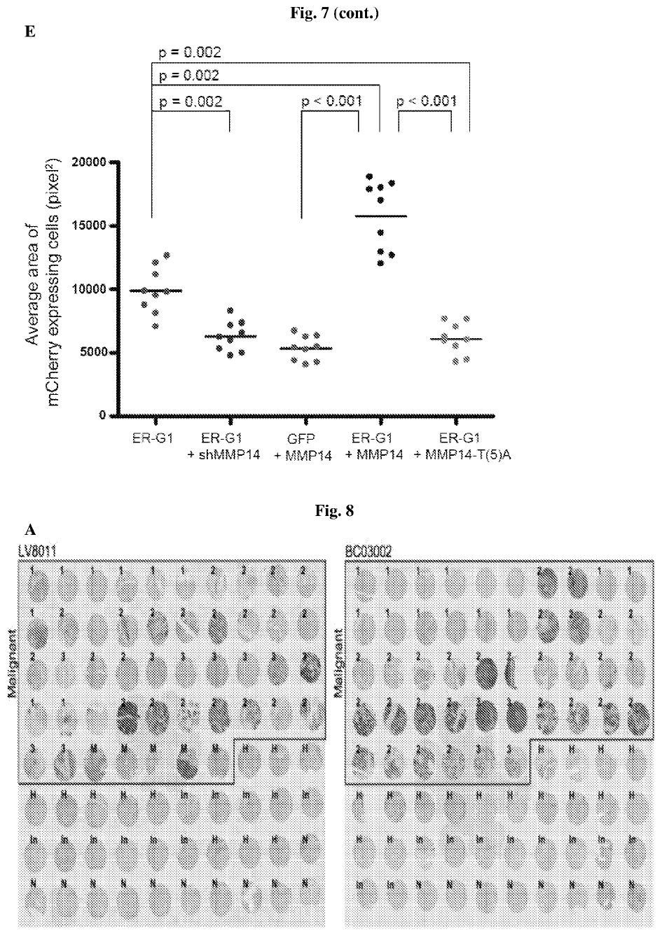

To test what role matrix metalloproteinase-14 (MMP14) plays in ER-G1-induced tumour growth, an shRNA against matrix metalloproteinase-14 (MMP14) (shMMP14) was co-expressed with ER-G1/NRas/shp53. Matrix metalloproteinase-14 (MMP14) expression was reduced by about 70% in liver tumours at 1 weeks post injection (wpi) (FIG. 13B). In these conditions, the rate of tumour development was reduced, with a median survival at 18 weeks versus 12 weeks for ER-G1/NRas/shp53 (FIG. 7A). CTC formation was reduced to levels comparable with that of the control mice (FIG. 7B). Late-stage tumours that formed in the presence of shMMP14 were less invasive, more differentiated, and produced much fewer metastases (FIG. 7C). These data indicate that the invasive phenotype induced by ER-G1 is dependent on matrix metalloproteinase-14 (MMP14) activity.

The proliferation rate of ER-G1-expressing tumours at 1 weeks post injection (wpi) was also observed to be significantly reduced by the knockdown of matrix metalloproteinase-14 (MMP14), indicating that this protease plays a promoting role from an early stage. To further test this notion, mice with hepatocytes expressing MMP14/ER-G1/NRas/shp53 (ER-G1+MMP14) were generated. Increased matrix metalloproteinase-14 (MMP14) levels led to a strong acceleration of proliferation at 7 days post-injection (dpi) (FIGS. 7D and 7E). This acceleration was dependent on enhanced glycosylation, since the expression of matrix metalloproteinase-14 (MMP14) in the absence of ER-G1 (GFP+MMP14) stimulated much less proliferation. Glycosylation of matrix metalloproteinase-14 (MMP14) itself is required, as indicated by the fact that expression of MMP14-T(5)A in the ER-G1/NRas/shp53 context did not stimulate growth. In tumours expressing recombinant matrix metalloproteinase-14 (MMP14), most of the protein appears cytoplasmic, with some staining at the cell surface (FIG. 13C). Similarly to the result in cell lines, no clear change in matrix metalloproteinase-14 (MMP14) subcellular distribution could be attributed to the glycosylation status.

Altogether, these results show that activation by ER-specific glycosylation promotes tumour growth at least partially through the promotion of matrix metalloproteinase-14 (MMP14) activity.

In summary, the relocation of GALNTs from the Golgi to the ER results in increased Tn levels. A cytoplasmic (i.e., ER-like) Tn pattern was found in virtually all human and mouse tumours where the glycan is increased. The glycosylation of the ER-resident protein PDIA4 is another hallmark of this relocation, and was up-regulated in 4/4 late-stage and 2/3 early-stage mouse tumours and increased in most human tumours. Since GALNT relocation is a highly regulated event, GALA must endow tumour cells with a competitive advantage, wherein GALA accelerates tumour growth from the first cell division events.

In addition to PDIA4, Vicia villosa lectin (VVL) immunoblotting and the current knowledge of GALNT substrates suggest that the ER localization of GALNTs stimulates the glycosylation of multiple substrates, potentially activating multiple factors favouring tumour growth. To note, ER-G1 tumours seemed to accumulate significantly more ECM than the control tumours.

This study shows that the members of the GALNTs relocation process and the ER-localized initiation of O-glycosylation enable tumour growth, and can be used for detection and characterisation.

### Description of SEQ ID NOS

Table 1 below details the SEQ ID NOs referenced herein and their corresponding sequences. A brief description of the sequences is also provided.

TABLE 1
Description of SEQ ID.

| SEQ ID | | |
|---|---|---|
| NO | Sequence | Description |
| 1 | cgtcaccctt ccagaaat | Forward primer that binds to GALNT sequences |
| 2 | ccactgcaaa gcttcttc | Reverse primer that binds to GALNT sequences |
| 3 | gctttggcga ggtgtggat | Forward primer that binds to SRC sequences |
| 4 | acatcgtgcc aggcttcag | Reverse primer that binds to SRC sequences |
| 5 | gtgagggaga gtgagaccac aaa | Forward primer that binds to Src sequences |
| 6 | ggcattgtcg aagtcggata c | Reverse primer that binds to Src sequences |
| 7 | gaggcaagga ctttgagcaa | Forward primer that binds to GBF1 sequences |
| 8 | tctgctcctc aggcattaca | Reverse primer that binds to GBF1 sequences |
| 9 | gctgcccacc ccaaatg | Forward primer that binds to Gbf1 sequences |
| 10 | tgaagggcac accaccagta | Reverse primer that binds to Gbf1 sequences |
| 11 | gcttaagctg ggtgagatcg | Forward primer that binds to ARF1 sequences |
| 12 | gtcccacaca gtgaagctga | Reverse primer that binds to ARF1 sequences |
| 13 | gcgccactac ttccagaaca c | Forward primer that binds to Arf1 sequences |
| 14 | gctctctgtc attgctgtcc acta | Reverse primer that binds to Arf1 sequences |
| 15 | ctgattctca aattccacgt cact | Forward primer that binds to GALNT1 sequences |
| 16 | gacactgatt cgtttccaca tttc | Reverse primer that binds to GALNT1 sequences |
| 17 | gacttcctgc tggtgacgtt ct | Forward primer that binds to Galnt1 sequences |
| 18 | ccccatttct ccaggacctt | Reverse primer that binds to Galnt1 sequences |
| 19 | gacgcctgag cagagaaggt | Forward primer that binds to GALNT2 sequences |
| 20 | cagcccacca gcaatcatg | Reverse primer that binds to GALNT2 sequences |
| 21 | cgcagcggtg ccttct | Forward primer that binds to Galnt2 sequences |
| 22 | gctccagcct gctctgaata tt | Reverse primer that binds to Galnt2 sequences |
| 23 | ccacgttgct tagaactgtc c | Forward primer that binds to GALNT3 |
| | | sequences |
| 24 | ccaaaatgat ttccttcagc a | Reverse primer that binds to GALNT3 |
| | | sequences |
| 25 | tgaaggagat cattttggtg gat | Forward primer that binds to Galnt3 sequences |
| 26 | ttcctccagc ttttcatgca | Reverse primer that binds to Galnt3 sequences |
| 27 | gtctgattgg ggccacttt | Forward primer that binds to GALNT4 sequences |
| 28 | cagccaaccg gaattacact | Reverse primer that binds to GALNT4 sequences |
| 29 | tctttcaggg tttggcagtg t | Forward primer that binds to Galnt4 sequences |
| 30 | gcccacgtgc gagcat | Reverse primer that binds to Galnt4 sequences |
| 31 | caataacctc ccaaccacca | Forward primer that binds to GALNT5 |
| | | sequences |
| 32 | ggaggagagc gattgatgac | Reverse primer that binds to GALNT5 sequences |
| 33 | cagtggacag agccattgaa ga | Forward primer that binds to Galnt5 sequences |
| 34 | tgggaggtca ttgtgaacta gttg | Reverse primer that binds to Galnt5 sequences |
| 35 | cgcaaagcag ctgtgtctac | Forward primer that binds to GALNT6 |
| | | sequences |
| 36 | tggctattct tgccagtgaa | Reverse primer that binds to GALNT6 |
| | | sequences |
| 37 | gcagaggtgc tcacgttcct | Forward primer that binds to Galnt6 sequences |
| 38 | ctccagccag ccgtgaaa | Reverse primer that binds to Galnt6 sequences |
| 39 | tgcattgata gcatgggaaa | Forward primer that binds to GALNT7 |
| | | sequences |
| 40 | gtggcagggt cctagttcaa | Reverse primer that binds to GALNT7 |
| | | sequences |
| 41 | ttggcgcaca gaaggctaa | Forward primer that binds to Galnt7 sequences |
| 42 | cacctcacag tgggcatcaa | Reverse primer that binds to Galnt7 sequences |
| 43 | tccactcttg aagccactcc | Forward primer that binds to GALNT8 |
| | | sequences |
| 44 | ccctgatcca agcagacatt | Reverse primer that binds to GALNT8 |
| | | sequences |
| 45 | ccattataca acgggccatc | Forward primer that binds to Galnt8 sequences |
| 46 | tgagctgaaa tcatccacca | Reverse primer that binds to Galnt8 sequences |
| 47 | aacgtgtacc cggagatgag | Forward primer that binds to GALNT9 |
| | | sequences |
| 48 | tccctggtcc agacagtagg | Reverse primer that binds to GALNT9 |
| | | sequences |
| 49 | agccatcctc tacccctgtc at | Forward primer that binds to Galnt9 sequences |
| 50 | caggagacct tcggcactgt a | Reverse primer that binds to Galnt9 sequences |
| 51 | tggatggatg agtacgcag a | Forward primer that binds to GALNT10 |
| | | sequences |
| 52 | gctttttctg gactgcgaca | Reverse primer that binds to GALNT10 |
| | | sequences |
| 53 | ctggcataac aaggaggcta tca | Forward primer that binds to Galnt10 sequences |
| 54 | ggcttcccct gttctccata t | Reverse primer that binds to Galnt10 sequences |
| 55 | acccaaagtc cttcaacgtg | Forward primer that binds to GALNT11 |
| | | sequences |
| 56 | gcatttgttg gtctggaggt | Reverse primer that binds to GALNT11 |
| | | sequences |
| 57 | agagtcctgc agcgtggaa | Forward primer that binds to Galnt11 sequences |
| 58 | ctgggccacc aggcat | Reverse primer that binds to Galnt11 sequences |
| 59 | tgaagcctgg tcaactctcc | Forward primer that binds to GALNT1 2 |
| | | sequences |
| 60 | gatatccggg gatgtctcaa | Reverse primer that binds to GALNT1 2 |
| | | sequences |
| 61 | gggatgggtc agaaccagtt t | Forward primer that binds to Galnt1 2 sequences |
| 62 | tggcgggtgt tatagcgtat t | Reverse primer that binds to Galnt1 2 sequences |
| 63 | gaagcttgga gcactctcct t | Forward primer that binds to GALNT1 3 |
| | | sequences |
| 64 | tggggaacga tttatcacac t | Reverse primer that binds to GALNT1 3 |
| | | sequences |
| 65 | ggctgtgctt attccaaaag atg | Forward primer that binds to Galnt1 3 sequences |
| 66 | gccatgaggt taaactgatt gattt | Reverse primer that binds to Galnt1 3 sequences |
| 67 | ctaaagttga gcccctgtgc | Forward primer that binds to GALNT14 |
| | | sequences |
| 68 | ccatacctgg gactttgcat | Reverse primer that binds to GALNT14 |
| | | sequences |
| 69 | cagaaagctt tgcgcctaga c | Forward primer that binds to Galnt14 sequences |
| 70 | ccctccggct atgattgga | Reverse primer that binds to Galnt14 sequences |
| 71 | caaccagctg gagagtgaca | Forward primer that binds to GALNTL1 |
| | | sequences |
| 72 | ggtccgagga gtaggacaca | Reverse primer that binds to GALNTL1 |
| | | sequences |
| 73 | tgtgacagga acaccctcaa | Forward primer that binds to Galnt11 sequences |
| 74 | gctgacaggt acgccttctc | Reverse primer that binds to Galnt11 sequences |
| 75 | cttccaggag aatgggatga | Forward primer that binds to GALNTL2 |
| | | sequences |
| 76 | ttgttttctt gcaccacagc | Reverse primer that binds to GALNTL2 |
| | | sequences |
| 77 | tacaagtggc ctgcctacag | Forward primer that binds to Galnt12 sequences |
| 78 | gcctcatcat ggaagcagag | Reverse primer that binds to Galnt12 sequences |
| 79 | cagcgtgtac ccagagatga | Forward primer that binds to GALNTL4 |
| | | sequences |
| 80 | cagcactcca taggcaatga | Reverse primer that binds to GALNTL4 |
| | | sequences |
| 81 | gctggaccac ttggagaatg | Forward primer that binds to Galnt14 sequences |
| 82 | gcaggagcct cttggatatg | Reverse primer that binds to Galnt14 sequences |
| 83 | tggatttttg gggaagagaa | Forward primer that binds to GALNTL5 |
| | | sequences |
| 84 | agagttggcc tccacacatc | Reverse primer that binds to GALNTL5 |
| | | sequences |
| 85 | ccaaggattg aggcgatatg | Forward primer that binds to Galnt15 sequences |
| 86 | tctctctcga tgcccagtct | Reverse primer that binds to Galnt15 sequences |
| 87 | acaacagccc cgttacactc | Forward primer that binds to GALNTL6 |
| | | sequences |
| 88 | tgttgctcac aggatggaa | Reverse primer that binds to GALNTL6 |
| | | sequences |
| 89 | atggctcggt tttccaaagt | Forward primer that binds to Galnt16 sequences |
| 90 | cggatgagac cttccctttt | Reverse primer that binds to Galnt16 sequences |
| 91 | ccttgtaaaa cccaaagatg tgagt | Forward primer that binds to Cl GALT1C1 |
| | | sequences |
| 92 | tgtcacagtg tttggtccaa gtc | Reverse primer that binds to Cl GALT1C1 |
| | | sequences |
| 93 | acgccggagt atttgcagaa | Forward primer that binds to C1galt1c1 |
| | | sequences |
| 94 | ccaacggatt tggtattaaa caca | Reverse primer that binds to C1galt1c1 |
| | | sequences |
| 95 | cgtcaccctt ccagaaat | Forward primer that binds to exogenous Galnt1- |
| | | GFP sequences |
| 96 | ccactgcaaa gcttcttc | Reverse primer that binds to exogenous Galnt1- |
| | | GFP sequences |

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

### Materials and Methods

### Experimental Model and Subject Details

### Cell Lines

HepG2 cells (male) were obtained from the American Type Culture Collection (ATCC) and were maintained in DMEM with 15% fetal bovine serum (FBS). All cell lines were grown at 37° C. in a 10% CO2 incubator.

### Animal Study

Six to eight week-old C57BL/6J male mice were obtained from Biological Resource Centre (BRC, Biomedical Sciences Institute, A*STAR). For hydrodynamic tail-vein injection, mice were kept in Tailveiner Restrainer (Braintree Scientific Inc., US) and injected via the lateral tail vein in 58 seconds using 27-gauge needles with a volume of solution corresponding to 10% their body weight. Each animal received 15 μg of transposase-encoding plasmid (pPGK-SB13), 30 μg of pT2/PGK/mCherry-Nras plasmid and 15 μg of pT2/shp53/PGK plasmid with the gene of interest (GOI). Plasmids were prepared using EndoFree Maxi Kit (Qiagen). DNA was suspended in Lactated Ringer's Injection (Baxter). Mice were monitored twice weekly for general health and tumour burden. Mice were euthanized and necropsied when the tumour size was estimated as 1-2.0 cm diameter or more by palpation. Liver tumours seen grossly were saved for histopathologic examination and molecular analysis. Mice were otherwise euthanized when moribund and full necropsies were performed. Tissues were snap-frozen or fixed in 10% formalin solution (Sigma Aldrich) and paraffin-embedded. For histology, 5 μm sections were processed for hematoxylin and eosin (H&E) staining. Histological classification of hepatic lesions and hepatocellular carcinoma (HCC) were reviewed by a pathologist based on published histological criteria. All animal experiments were performed in compliance with the Institutional Animal Care and Use Committee guidelines approved by Biological Resource Centre (BRC, Biomedical Sciences Institute, A*STAR).

### Human Tumour Microarrays

Human tumour microarrays BC03002 and LV8011 were purchased from US Biomax, Inc. (Rockville, Md.). The TMAs contain liver disease spectrum (hepatocellular carcinoma progression) with clear clinical stage and pathology grade. Please see http://www.biomax.us/tissue-arrays/Liver/LV8011, and http://www.biomax.us/tissue-arrays/Liver/BC03002 for details on hematoxylin and eosin (H&E)-stained images and classification of the tumour cores shown in FIG. 8A. Patient informed consent and approval was obtained by US Biomax, Inc, samples were anonymized and use was in accordance with the Human Biological Research Act of Singapore.

### Human Samples for Gene Expression Analysis

Human liver samples were obtained from patients undergoing curative resection for hepatocellular carcinoma (HCC) from 1991 to 2009 at the National Cancer Centre (NCC), Singapore. The collection of tumour and adjacent normal liver tissues and use for research was approved by NCC Institutional Review Board, Singapore. Written informed consent was obtained from all participating patients and all clinical and histopathological data provided to the researchers were rendered anonymous. Patient demographics and clinical descriptions have been reported in previous studies.

### Human Samples for Protein Analysis

Human liver samples were obtained from hepatocellular carcinoma (HCC) patients from 2014 to 2015 at the National Cancer Centre, Singapore. Cancerous and the corresponding distant noncancerous liver tissues were obtained from patients who underwent surgical resection as curative treatment for hepatocellular carcinoma (HCC). All tissue samples employed in this study were approved and provided by the Tissue Repository of the National Cancer Centre Singapore and conducted in accordance with the policies of its Ethics Committee. Informed consent was obtained from all participating patients and all clinical and histopathologic data provided to the researchers were rendered anonymous. All tissues were immediately snap frozen in liquid nitrogen until use. Information on the human hepatocellular carcinoma (HCC) patients can be found in Table 2 below.

TABLE 2
Information on the human hepatocellular
carcinoma (HCC) patients.

| LCFG | | | | Viral |
|---|---|---|---|---|
| Patient ID. | Gender | Age | Race | Status |
| F0009 | M | 69 | Chinese | HBV |
| F0012 | M | 84 | Chinese | Nil |
| F0016 | M | 58 | Chinese | HBV |
| F0017 | M | 78 | Chinese | Nil |
| F0019 | F | 56 | Chinese | HBV |
| F0022 | M | 71 | Chinese | Nil |
| F0025 | M | 75 | Chinese | Nil |
| F0026 | F | 74 | Chinese | Nil |
| F0028 | M | 64 | Indian | Nil |
| F0031 | M | 57 | Others | HCV |
| F0034 | M | 74 | Chinese | HBV |
| F0036 | F | 71 | Chinese | HBV |
| F0037 | M | 74 | Chinese | Nil |
| F0038 | F | 47 | Others | HCV |
| F0039 | F | 74 | Chinese | Nil |
| F0040 | M | 30 | Chinese | HBV |
| F0042 | F | 57 | Chinese | HBV |
| F0046 | F | 78 | Chinese | HBV |
| F0049 | M | 58 | Chinese | Nil |
| F0052 | M | 60 | Chinese | Nil |
| F0074 | F | 80 | Chinese | Nil |

### Method Details

### Vector Cloning

To construct the sleeping beauty vector, the vector pT2/shp53/GFP4 was digested with XhoI and ligated to a 2176-bp XhoI/SaII-synthesized fragment by Genscript USA Inc. The fragment contains sequences of shp53, the phosphoglycerate kinase (PGK) promoter, followed by EGFP and multiple cloning sites (MCS) to facilitate cloning. The resultant vector, named pT2/shp53/PGK-EGFP, was used to insert different genes of interest (GOIs). Mouse Galnt1 (NM_013814) was used in this study. Galnt1 (or Golgi-G1), ER-G1 (fused to an ER signal sequence from human growth hormone), ER-G1 with catalytic domain mutations D156Q, D209N and H211D to block substrate and manganese binding were synthesized by GenScript USA Inc. These GOIs were then cloned into the vector pT2/shp53/PGK-EGFP by AvrII sites, and fused to EGFP at the C-terminus. Another vector, pT/Caggs-NRASV12, was cut with EcoRV/XhoI to remove the CAGG promoter and ligated to a 1884-bp EcoRV/SaII fragment harboring a PGK promoter controlling the expression of mCherry-fused to human NRASG12V. The resultant vector is named pT2/PGK/mCherry-Nras. The pPGK-SB13 containing a version of the SB10 transposase was used in this study. The synthesized shMMP14 coding sequences were inserted into pT2/PGK/mCherry-Nras by two BgIII sites to obtain the pT2/shMMP14/PGK/mCherry-Nras construct. To generate pT2/PGK/mCherry-Nras-2A-MMP14-WT and pT2/PGK/mCherry-Nras-2A-MMP14-T(5)A vectors, human MMP14 wild-type and mutants containing 2A self-cleaving sequences were gene synthesized (Genscript), then cloned into vector pT2/PGK/mCherry-Nras by two SacII sites. The following vectors have been deposited at Addgene. ID #100974 for pT2/PGK/mCherry-Nras; ID #100975 for pT2/shp53/PGK-EGFP; ID #100976 for pT2/shp53/PGK/Golgi G1; ID #100977 for pT2/shp53/PGK/ER-G1 and ID #100978 for pT2/shp53/PGK/ER-G1Δcat.

To construct the pLENTI6.3 vectors, human GALNT1 (NM_020474) and human MMP14 (NM_004995) wild-type and mutants were gene synthesized (Genscript) and cloned into pDONR221 entry vector (ThermoFisher Scientific). The entry clones were then subcloned into the respective pLENTI6.3 destination vectors using gateway LR cloning reaction. See also Table 3 for list of plasmids used.

TABLE 3
List of plasmids and primers used.

| Reagent or Resource | Source | Identifier |
|---|---|---|
| ON-TARGETplus MMP14 siRNA (MMP14 OnT) | GE Healthcare | Cat# L-004145- |
| | Dharmacon | 00-0005 |
| siGenome MMP14 siRNA (MMP14 siG) | GE Healthcare | Cat# M- |
| | Dharmacon | 004145-00- |
| | | 0005 |
| SRC | Present | N/A |
| F: GCTTTGGCGAGGTGTGGAT (SEQ ID NO. 3); | application | |
| R: ACATCGTGCCAGGCTTCAG (SEQ ID NO. 4) | | |
| Src | Present | N/A |
| F: GTGAGGGAGAGTGAGACCACAAA (SEQ ID NO. 5); | application | |
| R: GGCATTGTCGAAGTCGGATAC (SEQ ID NO. 6) | | |
| GBF1 | Present | N/A |
| F: GAGGCAAGGACTTTGAGCAA (SEQ ID NO. 7); | application | |
| R: TCTGCTCCTCAGGCATTACA (SEQ ID NO. 8) | | |
| Gbf1 | Present | N/A |
| F: GCTGCCCACCCCAAATG (SEQ ID NO. 9); | application | |
| R: TGAAGGGCACACCACCAGTA (SEQ ID NO. 10) | | |
| ARF1 | Present | N/A |
| F: GCTTAAGCTGGGTGAGATCG (SEQ ID NO. 11); | application | |
| R: GTCCCACACAGTGAAGCTGA (SEQ ID NO. 12) | | |
| Arf1 | Present | N/A |
| F: GCGCCACTACTTCCAGAACAC (SEQ ID NO. 13); | application | |
| R: GCTCTCTGTCATTGCTGTCCACTA (SEQ ID NO. | | |
| 14) | | |
| GALNT1 | Present | N/A |
| F: CTGATTCTCAAATTCCACGTCACT (SEQ ID NO. | application | |
| 15); | | |
| R: GACACTGATTCGTTTCCACATTTC (SEQ ID NO. 16) | | |
| Galnt1 | Present | N/A |
| F: GACTTCCTGCTGGTGACGTTCT (SEQ ID NO. 17); | application | |
| R: CCCCATTTCTCCAGGACCTT (SEQ ID NO. 18) | | |
| GALNT2 | Present | N/A |
| F: GACGCCTGAGCAGAGAAGGT (SEQ ID NO. 19); | application | |
| R: CAGCCCACCAGCAATCATG (SEQ ID NO. 20) | | |
| Galnt2 | Present | N/A |
| F: CGCAGCGGTGCCTTCT (SEQ ID NO. 21); | application | |
| R: GCTCCAGCCTGCTCTGAATATT (SEQ ID NO. 22) | | |
| GALNT3 | Present | N/A |
| F: CCACGTTGCTTAGAACTGTCC (SEQ ID NO. 23); | application | |
| R: CCAAAATGATTTCCTTCAGCA (SEQ ID NO. 24) | | |
| Galnt3 | Present | N/A |
| F: TGAAGGAGATCATTTTGGTGGAT (SEQ ID NO. 25); | application | |
| R: TTCCTCCAGCTTTTCATGCA (SEQ ID NO. | 26) | |
| GALNT4 | Present | N/A |
| F: GTCTGATTGGGGCCACTTT (SEQ ID NO. 27); | application | |
| R: CAGCCAACCGGAATTACACT (SEQ ID NO. 28) | | |
| Galnt4 | Present | N/A |
| F: TCTTTCAGGGTTTGGCAGTGT (SEQ ID NO. 29); | application | |
| R: GCCCACGTGCGAGCAT (SEQ ID NO. 30) | | |
| GALNT5 | Present | N/A |
| F: CAATAACCTCCCAACCACCA (SEQ ID NO. 31); | application | |
| R: GGAGGAGAGCGATTGATGAC (SEQ ID NO. 32) | | |
| Galnt5 | Present | N/A |
| F: CAGTGGACAGAGCCATTGAAGA (SEQ ID NO. 33); | application | |
| R: TGGGAGGTCATTGTGAACTAGTTG (SEQ ID NO. | | |
| 34) | | |
| GALNT6 | Present | N/A |
| F: CGCAAAGCAGCTGTGTCTAC (SEQ ID NO. 35); | application | |
| R: TGGCTATTCTTGCCAGTGAA (SEQ ID NO. 36) | | |
| Galnt6 | Present | N/A |
| F: GCAGAGGTGCTCACGTTCCT (SEQ ID NO. 37); | application | |
| R: CTCCAGCCAGCCGTGAAA (SEQ ID NO. 38) | | |
| GALNT7 | Present | N/A |
| F: TGCATTGATAGCATGGGAAA (SEQ ID NO. 39); | application | |
| R: GTGGCAGGGTCCTAGTTCAA (SEQ ID NO. 40) | | |
| Galnt7 | Present | N/A |
| F: TTGGCGCACAGAAGGCTAA (SEQ ID NO. 41); | application | |
| R: CACCTCACAGTGGGCATCAA (SEQ ID NO. 42) | | |
| GALNT8 | Present | N/A |
| F: TCCACTCTTGAAGCCACTCC (SEQ ID NO. 43); | application | |
| R: CCCTGATCCAAGCAGACATT (SEQ ID NO. 44) | | |
| Galnt8 | Present | N/A |
| F: CCATTATACAACGGGCCATC (SEQ ID NO. 45); | application | |
| R: TGAGCTGAAATCATCCACCA (SEQ ID NO. 46) | | |
| GALNT9 | Present | N/A |
| F: AACGTGTACCCGGAGATGAG (SEQ ID NO. 47); | application | |
| R: TCCCTGGTCCAGACAGTAGG (SEQ ID NO. 48) | | |
| Galnt9 | Present | N/A |
| F: AGCCATCCICTACCCCTGICAT (SEQ ID NO. 49); | application | |
| R: CAGGAGACCTTCGGCACTGTA (SEQ ID NO. 50) | | |
| GALNT10 | Present | N/A |
| F: TGGATGGATGAGTACGCAGA (SEQ ID NO. 51); | application | |
| R: GCTTTTTCTGGACTGCGACA (SEQ ID NO. 52) | | |
| Galnt10 | Present | N/A |
| F: CTGGCATAACAAGGAGGCTATCA (SEQ ID NO. 53); | application | |
| R: GGCTTCCCCTGTTCTCCATAT (SEQ ID NO. 54) | | |
| GALNT11 | Present | N/A |
| F: ACCCAAAGTCCTTCAACGTG (SEQ ID NO. 55); | application | |
| R: GCATTTGTTGGTCTGGAGGT (SEQ ID NO. 56) | | |
| Galnt11 | Present | N/A |
| F: AGAGTCCTGCAGCGTGGAA (SEQ ID NO. 57); | application | |
| R: CTGGGCCACCAGGCAT (SEQ ID NO. 58) | | |
| GALNT12 | Present | N/A |
| F: TGAAGCCTGGTCAACTCTCC (SEQ ID NO. 59); | application | |
| R: GATATCCGGGGATGTCTCAA (SEQ ID NO. 60) | | |
| Galnt12 | Present | N/A |
| F: GGGATGGGTCAGAACCAGTTT (SEQ ID NO. 61); | application | |
| R: TGGCGGGTGTTATAGCGTATT (SEQ ID NO. 62) | | |
| GALNT13 | Present | N/A |
| F: GAAGCTTGGAGCACTCTCCTT (SEQ ID NO. 63); | application | |
| R: TGGGGAACGATTTATCACACT (SEQ ID NO. 64) | | |
| Galnt13 | Present | N/A |
| F: GGCTGTGCTTATTCCAAAAGATG (SEQ ID NO. 65); | application | |
| R: GCCATGAGGTTAAACTGATTGATTT (SEQ ID NO. | | |
| 66) | | |
| GALNT14 | Present | N/A |
| F: CTAAAGTTGAGCCCCTGTGC (SEQ ID NO. 67); | application | |
| R: CCATACCTGGGACTTTGCAT (SEQ ID NO. 68) | | |
| Galnt14 | Present | N/A |
| F: CAGAAAGCTTTGCGCCTAGAC (SEQ ID NO. 69); | application | |
| R: CCCTCCGGCTATGATTGGA (SEQ ID NO. 70) | | |
| GALNTL1 | Present | N/A |
| F: CAACCAGCTGGAGAGTGACA (SEQ ID NO. 71); | application | |
| R: GGTCCGAGGAGTAGGACACA (SEQ ID NO. 72) | | |
| Galntl1 | Present | N/A |
| F: TGTGACAGGAACACCCTCAA (SEQ ID NO. 73); | application | |
| R: GCTGACAGGTACGCCTTCTC (SEQ ID NO. 74) | | |
| GALNTL2 | Present | N/A |
| F: CTTCCAGGAGAATGGGATGA (SEQ ID NO. 75); | application | |
| R: TTGTTTTCTTGCACCACAGC (SEQ ID NO. 76) | | |
| Galntl2 | Present | N/A |
| F: TACAAGTGGCCTGCCTACAG (SEQ ID NO. 77); | application | |
| R: GCCTCATCATGGAAGCAGAG (SEQ ID NO. 78) | | |
| GALNTL4 | Present | N/A |
| F: CAGCGTGTACCCAGAGATGA (SEQ ID NO. 79); | application | |
| R: CAGCACTCCATAGGCAATGA (SEQ ID NO. 80) | | |
| Galntl4 | Present | N/A |
| F: GCTGGACCACTTGGAGAATG (SEQ ID NO. 81); | application | |
| R: GCAGGAGCCTCTTGGATATG (SEQ ID NO. 82) | | |
| GALNTL5 | Present | N/A |
| F: TGGATTTTTGGGGAAGAGAA (SEQ ID NO. 83); | application | |
| R: AGAGTTGGCCTCCACACATC (SEQ ID NO. 84) | | |
| Galntl5 | Present | N/A |
| F: CCAAGGATTGAGGCGATATG (SEQ ID NO. 85); | application | |
| R: TCTCTCTCGATGCCCAGTCT (SEQ ID NO. 86) | | |
| GALNTL6 | Present | N/A |
| F: ACAACAGCCCCGTTACACTC (SEQ ID NO. 87); | application | |
| R: TGTTGCTCACAGGATGGAA (SEQ ID NO. 88) | | |
| Galntl6 | Present | N/A |
| F: ATGGCTCGGTTTTCCAAAGT (SEQ ID NO. 89); | application | |
| R: CGGATGAGACCTTCCCTTTT (SEQ ID NO. 90) | | |
| C1GALT1C1 | Present | N/A |
| F: CCTTGTAAAACCCAAAGATGTGAGT (SEQ ID NO. | application | |
| 91); | | |
| R: TGTCACAGTGTTTGGTCCAAGTC (SEQ ID NO. 92) | | |
| C1galt1c1 | Present | N/A |
| F: ACGCCGGAGTATTTGCAGAA (SEQ ID NO. 93); | application | |
| R: CCAACGGATTTGGTATTAAACACA (SEQ ID NO. | | |
| 94) | | |
| Exogenous Galnt1-GFP | Present | N/A |
| F: CGTCACCCTTCCAGAAAT (SEQ ID NO. 95); | application | |
| R: CCACTGCAAAGCTTCTTC (SEQ ID NO. 96) | | |

Quantitative RT-PCR (qRT-PCR)

Total RNA was extracted using TRIzol (Invitrogen) and reverse transcribed to cDNA using the SuperScript III cDNA Synthesis Kit (Invitrogen) following the manufacturer's instructions. The Fluidigm BioMark real-time PCR system and 48.48 Microfluidic Dynamic Array were used for qRT-PCR analysis. Primer sequences were designed by Primer Express Software v3 and listed in Tables 1 and 3. For the specific target amplification (STA) pre-amplification reaction, each cDNA sample was pre-amplified with 200 nM pooled STA primer mix and Tagman PreAmp Master Mix (Applied Biosystems) in a 5 μl reaction, which was run for 14 cycles according to the manufacturer's protocol. To remove unincorporated primers, each sample was treated with Exonuclease I (ThermoFisher Scientific) following incubation at 37° C. for 30 minutes. For inactivation, the mix was in a second step, incubated at 80° C. for 15 minutes. At the end of the Exonuclease I treatment, the reactions were diluted 1:5 in TE buffer (pH 8.0) prior to use for qRT-PCR. The Fluidigm BioMark™ real-time PCR system and 48.48 Microfluidic Dynamic Arrays were employed for high-throughput qRT-PCR analysis. As volume per inlet is 5 μl, the 6 μl volume per inlet with overage was prepared. For the samples, 2.7 μl of each STA and ExoI-treated sample were mixed with 20×DNA Binding Dye Sample Loading Reagent (Fluidigm) and 2× SsoFast EvaGreen SuperMix with Low ROX (Bio-Rad). For the gene expression assays, 0.3 μl of mix primer pairs (100 uM) was added with 2× Assay Loading Reagent (Fluidigm) following the addition of 1×TE buffer to 6 μl volume. Prior to loading the samples and assays into the inlets, the chip was primed in the NanoFlex 4-IFC Controller. The samples and assays were then loaded into the inlets of the dynamic array. Following loading and mixing of the samples and assays into the chip by the IFC Controller, PCR was run with the following reactions conditions: 50° C. for 2 minutes, 95° C. for 10 minutes, followed by 40 cycles of 95° C. for 15 seconds and 60° C. for 60 seconds. Global threshold and linear baseline correction were automatically calculated for the entire chip. ATCB, GUSB and Atcb, Gusb were used as internal control genes in human and mouse samples, respectively. Fold change in expression of GOIs between liver tumour and adjacent non-tumour samples were calculated using the comparative cycle threshold Ct method following the formula: 2-ΔCt (tumour)/2-ΔCt (non-tumour). The -ΔCt data obtained from this calculation was used to generate the heatmap as well as supervised hierarchical clustering between samples by dChip software (www.dChip.org). Pearson correlation subtracted from unity was used as the distance metric, employing the centroid linkage method, which provides bounded distances in the range (−2, 2). p value threshold for function enrichment is <0.01. Significantly differentially expressed genes were identified in human and mouse liver tumours with fold-change ≥1.5 and p≤50.05 as compared to normal tissue (t-test).

### Immunohistochemistry (IHC)

Samples were de-paraffinized in Bond Dewax Solution and rehydrated through 100% ethanol to 1× Bond Wash Solution (Leica Biosystems). Samples were boiled for 40 minutes at 100° C. for antigen retrieval using Bond Epitope Retrieval Solution, then treated with 3% hydrogen peroxide for 15 minutes and incubated with 10% goat serum block for 30 min. Subsequent staining with Vicia villosa lectin (VVL)-Biotin (1:1000) was performed at room temperature for 60 min. After rinsing three times in Bond Wash Solution, samples were incubated with secondary Streptavidin-HRP antibody (1:200) at room temperature for 30 min. Signals indicating horseradish peroxidase (HRP-DAB) activity were visualised using Bond Refine Detection Kit (Leica) following the manufacturer's instructions. The nuclei were counterstained with hematoxylin for 5 min, dehydrated, and mounted for microscopic examination.

### Detection of Circulating Tumour Cells by FACs Analysis

Blood (300 μl) was collected from control, Golgi-G1 and ER-G1 mice at 3 to 4 months post-injection and treated with 10 ml of ammonium-chloride-potassium (ACK) lysis buffer (ThermoFisher Scientific) at room temperature to lyse red blood cells. Cell pellets were suspended in PBS containing 2 mM EDTA and 2% FBS, and analysed for the number of EGFP+ cells by flow cytometry (MoFlo XDP, Beckman Coulter). The data are presented as the percentage of EGFP+ cells from gated cells; approximately 100,000 cells were analysed at the time of acquisition.

Stimulation with Growth Factors

Before growth factor stimulation, HEK293T cells were washed twice using Dulbecco's phosphate-buffered saline (D-PBS) and serum starved in serum-free DMEM for at least 16 hours. Human recombinant EGF (100 ng/ml; Sigma-Aldrich) or mouse recombinant PDGF-bb (50 ng/ml; Invitrogen) were added for various durations before lysis.

### Western Blot Analysis

Harvested liver tissues were weighed and homogenized in ice-cold RIPA lysis buffer (50 mM Tris [pH 8.0, 4° C.], 200 mM NaCl, 0.5% NP-40 and complete protease inhibitor [Roche Applied Science]). The samples were lysed for 1 hour with constant agitation before clarification by centrifugation at 13000×g for 10 minutes at 4° C. To prepare the cells, the cell lines were washed twice with ice-cold D PBS, scraped in ice-cold RIPA lysis buffer, and lysed for 30 minutes with constant agitation before sample clarification. Clarified lysate protein concentrations were determined using Bradford reagent (Bio-Rad) before sample normalization for immunoprecipitation (IP) or sodium dodecyl sulfate polyacrylamide gel electrophoresis (SDS-PAGE) electrophoresis using 4-12% Bis-Tris NuPage gels at 200V for 60 min. Electrophoresed samples were transferred on nitrocellulose membranes and blocked using 3% BSA dissolved in TBST (50 mM Tris [pH 8.0, 4° C.], 150 mM NaCl, and 0.1% Tween-20) for 1 hour at room temperature. Membranes were then incubated with primary antibodies or biotinylated-Vicia villosa lectin (VVL) (0.2 μg/ml) overnight at 4° C. The next day, membranes were washed three times in TBST before the addition of secondary antibody conjugated with horseradish peroxidase (HRP) or streptavidin-HRP. Membranes were further washed three times with TBST before ECL exposure.

### Lectin Immunoprecipitation (IP)

Clarified cell/tissue lysates were incubated with Vicia villosa lectin (VVL)-conjugated beads for 2 hours at 4° C. The beads were washed at least three times with RIPA lysis buffer, before the precipitated proteins were eluted in 2×LDS sample buffer with 50 mM DTT by boiling at 95° C. for 10 minutes. For peanut agglutinin (PNA) and Datura stramonium Lectin (DSL) pulldowns, the cell lysates were incubated with biotinylated-PNA or -DSL lectins in lysis buffer supplemented with 2 mM CaCl₂ and MgCl₂ overnight at 4° C. The lectin-bound proteins were then IP with streptavidin beads for 2 hours at 4° C. before eluting by boiling in 2×LDS sample buffer with 50 mM DTT.

### GalNAz Metabolic Labelling

HepG2 cell lines were metabolically labelled with 200 μM GalNAz for 72 hours. Cells were lysed with RIPA lysis buffer and the clarified lysates were labelled with 250 μM of FLAG-phosphine overnight under constant agitation. The FLAG-GalNAz-labelled proteins were immunoprecipitated with FLAG antibody (Sigma Aldrich) for 1 hour and then incubated for 2 hours with protein G-Sepharose at 4° C. The IP samples were washed three times with lysis buffer and boiled in 2×LDS loading buffer at 95° C. for 10 minutes. Samples were resolved by sodium dodecyl sulfate polyacrylamide gel electrophoresis (SDS-PAGE) electrophoresis using 4-12% Bis-Tris NuPage gels at 200 V for 60 minutes before transfer on nitrocellulose membranes.

### Cell-Surface Biotinylation

HepG2 cell lines were transfected with the matrix metalloproteinase-14 (MMP14) mutants and grown to 95% confluence before cell-surface labelling with a cell-impermeable biotinylation reagent, Sulfo-NHS-SS-Biotin, for 30 minutes at 4° C. under constant agitation. The biotinylation process was quenched, and the cells were then harvested and lysed. The cell-surface proteins were isolated using the Pierce Cell Surface Protein Isolation Kit (ThermoFisher Scientific) following the manufacturers' instructions.

### Immunofluorescence (IF) Microscopy

Cells were seeded at 20,000 cells per well in a 96-well plate (Falcon) and incubated at 37° C. with 10% CO₂ overnight. The cells were fixed with 4% paraformaldehyde in D-PBS for 10 minutes, washed once with D-PBS, and then permeabilised with 0.2% Triton X-100 for a further 10 minutes. The cells were then stained with Helix pomatia lectin (HPL) and Hoechst 33342 diluted in 2% FBS in D-PBS for 20 minutes and washed three times for 5 minutes with D-PBS before high-throughput confocal imaging. Four sites per well were acquired sequentially with a 20× Plan Apo 0.75 NA objective on a laser-scanning confocal high-throughput microscope (ImageXpress Ultra, Molecular Devices).

For mouse samples, the slides required deparaffinization, antigen retrieval, and blocking steps before incubation with antibodies. Staining for Vicia villosa lectin (VVL)-biotin (4 μg/ml), Calnexin (1:100, Abcam, ab22595) and Hoescht (1:10,000) was performed overnight and counterstained with anti-rabbit Alexa Fluor 488 (1:1000) or Streptavidin-Alexa 594 (1:400) secondary antibodies for 30 minutes. Slides were counterstained with DAPI and then mounted (Vectashield) before confocal imaging.

### Matrix Metalloproteinases (MMP) Activity Assay

HepG2 cell lines or harvested liver tissues were lysed in ice cold lysis buffer (50 mM Tris [pH 8.0, 4° C.], 200 mM NaCl, 0.5% NP-40 alternative and complete protease inhibitor [Roche Applied Science]) for 1 hour with constant agitation before clarification of samples by centrifugation at 13,000×g for 10 minutes at 4° C. Total protein levels of each sample were measured using Bradford assay. 100 μg (HepG2 cell lysates) and 60 μg (liver tissue lysates) of total protein lysate were added to matrix metalloproteinase (MMP) Förster resonance energy transfer (FRET) peptide substrate solution (Abcam) that was prepared according to the manufacturers' protocol. The samples were measured on a plate reader (Excitation/Emission=540/590 nm) over intervals of 5 minutes to determine the cleavage of the peptide substrate. Three replicates were performed for each condition.

### Matrix Degradation Assay

HepG2 cells were seeded on either fluorescent red gelatin matrix or layered fluorescent red gelatin/collagen I matrix for 2 days. Gelatin was coupled to rhodamine by incubation with 5-carboxy-X-rhodamin succinimidyl ester (ThermoFisher Scientific) and coated onto sterile coverslips for 20 minutes. Coverslips were then fixed with 0.5% glutaraldehyde for 40 minutes (Electron Microscopy Sciences) and washed 3 times with 1×PBS. Layered red gelatin/collagen I coverslips were prepared by incubating 0.5 mg/ml collagen I (Corning) diluted in D-PBS for 4 hour at 37° C. on coated coverslips. Confocal images of rhodamine and nuclei channels were obtained using a confocal microscope (Zeiss LSM700) with a 10× or 20× objective. At least 30 images were acquired for each condition. The area of degradation was quantified using ImageJ software whereby the degradation area was delineated manually with the threshold bar. The degradation area was then normalized to the number of nuclei in each image.

### Cell Proliferation Assay

HepG2 cell lines were seeded in 24-well plate (50,000 cells per well) and incubated overnight at 37° C. for them to adhere. The plate was transferred into Incucyte system (Essen BioScience) for live imaging with phase contrast microscopy. 16 images per well, in triplicates, were taken every 6 hours for 7 days. The level of proliferation was then determined by measuring cell confluency at each time point, using Incucyte software. Three independent experimental replicates were performed.

### Quantification and Statistical Analysis

### Survival Analysis

Kaplan-Meier survival curves were computed by Prism4 (GraphPad). The log-rank test was used to compare significant differences in death rates between different mouse cohorts. Prism4 performed a Student's t-test for direct comparison between GFP (control) and other cohorts. Based on Bonferroni's correction for multiple comparisons, p values of 50.01 were considered statistically significant.

### Quantification of Immunohistochemical (IHC) Staining

All immunohistochemical (IHC) slides were scanned using a Leica SCN400 and viewed through Ariol-Slidepath Digital Imaging Hub (Leica Microsystems). Images captured by this system were used for quantification of Vicia villosa lectin (VVL) staining in human tumour cores and mouse liver tumours. The immunohistochemical (IHC) images were first converted to negative images by the Ariol System. ImageJ was used to calculate and subtract non-specific and counterstaining background from the whole TMAs. Corrected intensity measurements were divided by the total core area to generate the intensity per pixel per core. Final normalization to mean intensity per pixel per core from all normal tissue cores in each array was performed to enable direct comparison of Vicia villosa lectin (VVL) staining in the BC03002 and LV8011 arrays.

For the mice tumour sections, constant image calculator and subtract background were applied with ImageJ. At least three fields (diameter 200 μm) per section were used to measure Vicia villosa lectin (VVL) staining. The mean values for each tumour section were then normalized to the average area of control or normal liver sections. To quantify the area of Sleeping Beauty (SB) transposon-transformed cells in the livers of post-1 week-injected mice, the immunohistochemical (IHC) images of the mCherry-Nras staining were analysed using ImageJ. Images were first converted to 8-bit greyscale format and an automatic threshold was set to select the mCherry-stained areas. Small unstained areas within the stained cells were covered using the “fill holes” process. The area of each object above 500 pixel2 in size was measured, and the average area per object in each image was calculated. Please refer the FIGS. 9C and 9D for summarized workflow.

### Quantification of Immunofluorescence (IF) Staining

Image analysis was performed using MetaXpress software (version 3.1.0.89). For each well, total Helix pomatia lectin (HPL) staining intensity and nuclei number was quantified using the Transfluor HT application module in the software. Hundreds of cells from at least three wells per experiment were quantified. Three experimental replicates were performed.

To quantify degradation in matrix degradation assay, the area of degradation was quantified using ImageJ software. The degraded area was selected by adjusting the threshold and the total area of degradation in the image was measured. The degradation area was then normalized to the number of nuclei in each image. At least 30 images per condition were quantified from each experiment. Three independent experimental replicates were performed. Results are presented as the mean value and standard deviation (SD) unless stated otherwise. Statistical significance was measured using a Student's t-test assuming a two-tailed Gaussian distribution. Asterisks in figures denote statistical significance (*, p<0.05 or p<0.01; **, p<0.001; ***, p<0.0001).

### Quantification of Western Blot Bands

Image analysis was performed using ImageJ. To quantify the intensity of the band, the image was inverted to black background and a box was drawn over the band of interest. The mean intensity of the band within the box area was measured, taking into account the mean intensity of the background.

1. A method of detecting the presence or absence of cancer,
wherein the method comprises the steps of:
(i) obtaining a sample from a subject;
(ii) detecting a level of O-glycosylation of one or more endoplasmic reticulum (ER)-resident proteins in the sample obtained in step (i);
(iii) comparing the level of O-glycosylation of one or more endoplasmic reticulum (ER)-resident proteins in step (ii) with a level of O-glycosylation of one or more endoplasmic reticulum (ER)-resident proteins in a control group;
wherein an increase in the level of O-glycosylation of one or more endoplasmic reticulum (ER)-resident proteins present in the sample compared to the control group is indicative of the presence of cancer.

2. A method of determining the risk of a subject developing cancer, wherein the method comprises the steps of:
(i) obtaining a sample from a subject;
(ii) detecting a level of O-glycosylation of one or more endoplasmic reticulum (ER)-resident proteins in the sample;
(iii) comparing the level of O-glycosylation of one or more endoplasmic reticulum (ER)-resident proteins in step (ii) with a level of O-glycosylation of one or more endoplasmic reticulum (ER)-resident proteins in a control group;
wherein an at least 4-fold increase in the level of O-glycosylation of one or more endoplasmic reticulum (ER)-resident proteins present in the sample compared to the control group is indicative that the subject is suffering from cancer.

3. The method according to claim 1, wherein the detection of the level of O-glycosylation of one or more endoplasmic reticulum (ER)-resident proteins comprises contacting the sample with a monosaccharide-binding protein.

4. The method according to claim 1, wherein the one or more endoplasmic reticulum (ER)-resident proteins is selected from the group consisting of protein disulfide isomerase family A member 4 (PDIA4), calnexin (CANX), protein disulfide isomerase family A member 3 (PDIA3), Endoplasmic Reticulum Lectin 1 (ERLEC1), and heat shock 70 kDa protein 5 (glucose-regulated protein, 78 kDa) (HSPA5/GRP78/Bip).

5. The method according to claim 4, wherein the one or more endoplasmic reticulum (ER)-resident proteins is protein disulfide isomerase family A member 4 (PDIA4) and/or calnexin (CANX).

6. The method according to claim 3, wherein the monosaccharide-binding protein is an N-acetylgalactosamine binding protein.

7. The method according to claim 6, wherein the N-acetylgalactosamine binding protein is selected from the group consisting of Vicia villosa lectin (VVL), Helix pomatia lectin A (HPL), ricin (RCA), peanut agglutinin (PNA), and jacalin (AIL).

8. The method according to claim 7, wherein the N-acetylgalactosamine binding protein is either Vicia villosa lectin (VVL) or Helix pomatia lectin A (HPL).

9. The method of claim 1, wherein the cancer is selected from the group consisting of liver cancer, breast cancer, lung cancer, hepatocellular carcinoma (HCC), hepatocellular adenoma (HCA), fibrolamellar hepatocellular carcinoma (FHCC), hepatoblastoma, focal nodular hyperplasia (FNH), nodular regenerative hyperplasia, ductal carcinoma in situ (DCIS), Paget's disease of the breast, comedocarcinoma, invasive ductal carcinoma (IDC), intraductal papilloma, lobular carcinoma in situ (LCIS), invasive lobular carcinoma (ILC), medullary carcinoma, inflammatory breast cancer, non-small cell lung cancer (NSCLC), and small cell lung cancer (SCLC).

10. The method of claim 1, wherein the cancer is malignant.

11. The method of claim 1, wherein the control group is a disease-free group.

12. A method of determining the malignancy, grade, or staging of a cancer, the method comprising
(i) obtaining a sample from a subject;
(ii) detecting a level of O-glycosylation of one or more endoplasmic reticulum (ER)-resident proteins in the sample;
(iii) comparing the level of O-glycosylation of one or more endoplasmic reticulum (ER)-resident proteins in step (ii) with a level of O-glycosylation of one or more endoplasmic reticulum (ER)-resident proteins in a group defined for each grade of cancer.

13. A kit comprising:
(i) a monosaccharide-binding protein capable of binding to one or more O-glycosylated endoplasmic reticulum (ER)-resident proteins;
(ii) a detection agent capable of binding to the monosaccharide-binding protein and/or the one or more O-glycosylated endoplasmic reticulum (ER)-resident proteins; and
(iii) one or more standards, wherein each standard comprises an O-glycosylated endoplasmic reticulum (ER)-resident protein selected from the group consisting of protein disulfide isomerase family A member 4 (PDIA4), calnexin (CANX), protein disulfide isomerase family A member 3 (PDIA3), Endoplasmic Reticulum Lectin 1 (ERLEC1), and heat shock 70 kDa protein 5 (glucose-regulated protein, 78 kDa) (HSPA5/GRP78/Bip).

14. The kit according to claim 13, wherein the kit is used to determine a level of the one or more O-glycosylated endoplasmic reticulum (ER)-resident proteins in a sample and/or to compare a level of the one or more O-glycosylated endoplasmic reticulum (ER)-resident proteins a baseline level provided by the standard.

15. The kit according to claim 13, wherein the kit is an enzyme-linked immunosorbent assay (ELISA).

16. The kit of claim 15, wherein the ELISA kit comprises:
(a) a microwell plate;
(b) a sample diluent;
(c) a wash buffer;
(d) a substrate solution that can be detected using the detection agent; and
(e) a stop solution capable of reacting with the substrate solution and allowing visualisation.

17. The kit according to claim 4, wherein the one or more endoplasmic reticulum (ER)-resident protein is protein disulfide isomerase family A member 4 (PDIA4) and/or calnexin (CANX).
